(19) United States
(12) Patent Application Publication
(10) Pub. No.: US 2022/0106614 A1
(43) Pub. Date: Apr. 7, 2022

(54) DLX2 VECTOR

(71) Applicant: NeuExcell Therapeutics Inc., State College, PA (US)

(72) Inventor: Jie XU, State College, PA (US)

(73) Assignee: NeuExcell Therapeutics Inc., State College, PA (US)

(21) Appl. No.: 17/487,888

(22) Filed: Sep. 28, 2021

Related U.S. Application Data
(60) Provisional application No. 63/247,417, filed on Sep. 23, 2021.
(60) Provisional application No. 63/084,927, filed on Sep. 29, 2020.

(51) Int. Cl. C12N 15/86; A61K 48/00; A61P 25/28

(52) U.S. Cl. CPC C12N 15/86; A61K 48/0066; C12N 2830/85; C12N 2830/48; C12N 2830/50; A61P 25/28

(57) ABSTRACT

The present disclosure relates to AAV vectors, compositions, and methods related to converting glial cells to neurons by the use of a Dlx2 coding sequence in an AAV vector.

### CROSS-REFERENCE TO RELATED APPLICATION

This application is a U.S. patent application which claims the benefit and priority to U.S. Provisional Application Nos. 63/084,927 filed Sep. 29, 2020, and 63/247,417 filed Sep. 23, 2021, each of which are incorporated by reference in their entireties herein.

### INCORPORATION BY REFERENCE

A sequence listing contained in the file named P34836US02_SL.txt which is 19,859 bytes (measured in MS-Windows®) and created on Sep. 27, 2021, is filed electronically herewith and incorporated by reference in its entirety.

### FIELD OF THE INVENTION

The present disclosure includes methods and compositions using an AAV vector comprising a nucleic acid sequence encoding human Dlx2 to convert glial cells to neurons.

### BACKGROUND OF THE INVENTION

Neurons are often killed or damaged and unable to regenerate in subjects with a neurological condition or following an injury to the central nervous system (CNS) or peripheral nervous system (PNS).

Glial cells become reactive following an injury to the CNS or PNS such as a brain injury or neurological condition.

Currently there are no methods available to regenerate functional new neurons in human subjects having a neurological condition using adeno-associated viruses (AAVs).

### SUMMARY OF THE INVENTION

In one aspect, this disclosure provides, and includes, an adeno-associated virus (AAV) vector comprising a human distal-less homeobox 2 (hDlx2) sequence comprising the nucleic acid sequence of SEQ ID NO: 6, where the hDlx2 sequence is operably linked to regulatory elements comprising: (a) a glial fibrillary acid protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8 or a hGH polyadenylation signal comprising the nucleic acid sequence of SEQ ID NO: 13, or a bGH polyadenylation signal comprising the nucleic acid sequence of SEQ ID NO: 20.

In one aspect, this disclosure provides, and includes, an adeno-associated virus (AAV) vector comprising a nucleic acid coding sequence encoding a human distal-less homeobox 2 (hDlx2) protein comprising the amino acid sequence of SEQ ID NO: 10, where the coding sequence is operably linked to regulatory elements comprising: (a) a glial fibrillary acid protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; (e) and a SV40 polyadenylation signal with a nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, an adeno-associated virus (AAV) vector comprising a distal-less homeobox 2 (Dlx2) nucleic acid coding sequence encoding a Dlx2 protein, where the coding sequence is operably linked to regulatory elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human distal-less homeobox 2 (hDlx2) sequence having a nucleic acid sequence of SEQ ID NO: 6, and where the sequence is operably linked to regulatory elements comprising: (a) a human glial fibrillary acid protein (GFAP) promoter comprising a nucleic acid sequence selected from the group consisting of SEQ ID NOs: 3 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a composition comprising an adeno-associated-virus (AAV) vector for converting glial cells to functional neurons in a human, where the AAV vector comprises a nucleic acid coding sequence encoding a human distal-less homeobox 2 (hDlx2) protein comprising the amino acid sequence of SEQ ID NO: 10, and where the coding sequence is operably linked to regulatory elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a composition comprising an adeno-associated virus (AAV) vector for the treatment of a subject in need thereof, where the AAV vector comprises a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal.

In an aspect, this disclosure provides, and includes, a method of converting reactive astrocytes to functional neurons in a brain of a living human comprising: injecting an adeno-associated virus (AAV) into a subject in need thereof, where the AAV comprises a DNA vector construct comprising a human distal-less homeobox 2 (hDlx2) sequence comprising the nucleic acid sequence of SEQ ID NO: 6, where the sequence is operably linked to regulatory elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a method of converting reactive astrocytes to functional neurons in a brain of a living human comprising: injecting an adeno-associated virus (AAV) into a subject in need thereof, where the AAV comprises a DNA vector construct comprising a nucleic acid coding sequence encoding a human distal-less homeobox 2 (hDlx2) protein comprising the amino acid sequence of SEQ ID NO: 10, where the coding sequence is operably linked to expression control elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) and a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to the subject in need thereof.

In an aspect, this disclosure provides, and includes, an adeno-associated virus (AAV) vector comprising a human distal-less homeobox 2 (hDlx2) sequence comprising the nucleic acid sequence of SEQ ID NO: 6, where the hDlx2 sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, an adeno-associated virus (AAV) vector comprising a nucleic acid coding sequence encoding a human distal-less homeobox 2 (hDlx2) protein comprising the amino acid sequence of SEQ ID NO: 10, where said coding sequence is operably linked to regulatory elements comprising: (a) a glial fibrillary acidic protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal with a nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a composition comprising an adeno-associated virus (AAV) vector for converting glial cells to functional neurons in a human, where said AAV vector comprises a human distal-less homeobox 2 (hDlx2) sequence having a nucleic acid sequence of SEQ ID NO: 6, and where said sequence is operably linked to regulatory elements comprising: (a) a human glial fibrillary acidic protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a composition comprising an adeno-associated-virus (AAV) vector for converting glial cells to functional neurons in a human, where said AAV vector comprises a nucleic acid sequence encoding a distal-less homeobox 2 (hDlx2) protein comprising the amino acid coding sequence of SEQ ID NO: 10, and where said coding sequence is operably linked to regulatory elements comprising: (a) a human glial fibrillary acidic protein (GFAP) promoter comprising a nucleic acid sequence selected from the group consisting of SEQ ID NOs: 3,4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal sequence comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a method of converting reactive astrocytes to functional neurons in a brain of a living human comprising: injecting an adeno-associated virus (AAV) into a subject in need thereof, where said AAV comprises a DNA vector construct comprising a human distal-less homeobox 2 (hDlx2) sequence comprising the nucleic acid sequence of SEQ ID NO: 6, where said sequence is operably linked to regulatory elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a method of converting reactive astrocytes to functional neurons in a brain of a living brain comprising: injecting an adeno-associated virus (AAV) into a subject in need thereof, where said AAV comprises a DNA vector construct comprising a nucleic acid sequence encoding a human distal-less homeobox 2 (hDlx2) protein comprising the amino acid coding sequence of SEQ ID NO: 10, where said coding sequence is operably linked to expression control elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a map of a CE:Gfa681:Dlx2:WPRE:SV40.

FIG. 1B depicts a map of a EF-1α:Gfa681:Dlx2:WPRE:SV40.

FIG. 1C depicts a map of a CE:Gfa681:Dlx2:WPRE:hGH.

FIG. 1D depicts a map of a EF-1α:Gfa681: Dlx2:WPRE:hGH.

FIG. 2A depicts a map of a CE:Gfa1.6p:Dlx2:WPRE:SV40.

FIG. 2B depicts a map of a EF-1α:Gfa1.6p:Dlx2:WPRE:SV40.

FIG. 2C depicts a map of a CE: Gfa1.6p:Dlx2:WPRE:hGH.

FIG. 2D depicts a map of a EF-1α: Gfa1.6p:Dlx2:WPRE:hGH.

FIG. 3A depicts a map of a CE:Gfa2.2:Dlx2:WPRE:SV40.

FIG. 3B depicts a map of a EF-1α: Gfa2.2: Dlx2:WPRE:SV40.

FIG. 3C depicts a map of a CE: Gfa2.2:Dlx2:WPRE:hGH.

FIG. 3D depicts a map of a EF-1α: Gfa2.2:Dlx2:WPRE:hGH.

FIG. 4 depicts a map of a U6:shRNA1:H1:shRNA2:7SK:shRNA3.

FIG. 5A depicts a map of a U6:shRNA:CE:Gfa681:Dlx2:WPRE:SV40.

FIG. 5B depicts a map of a U6:shRNA: EF-1α:Gfa681:Dlx2:WPRE:SV40.

FIG. 5C depicts a map of a U6:shRNA:CE:Gfa1.6p:Dlx2:WPRE:SV40.

FIG. 5D depicts a map of a U6:shRNA: EF-1α: Gfa1.6p:Dlx2:WPRE:SV40.

FIG. 5E depicts a map of a U6:shRNA:CE:Gfa2.2:Dlx2:WPRE:SV40.

FIG. 5F depicts a map of a U6:shRNA: EF-1α: Gfa2.2:Dlx2:WPRE:SV40.

FIG. 6A depicts a map of a U6:shRNA:CE:Gfa681:Dlx2:WPRE:hGH.

FIG. 6B depicts a map of a U6:shRNA: EF-1α:Gfa681:Dlx2:WPRE:hGH.

FIG. 6C depicts a map of a U6:shRNA:CE:Gfa1.6p:Dlx2:WPRE:hGH.

FIG. 6D depicts a map of a U6:shRNA: EF-1α: Gfa1.6p:Dlx2:WPRE:hGH.

FIG. 6E depicts a map of a U6:shRNA:CE:Gfa2.2:Dlx2:WPRE:hGH.

FIG. 6F depicts a map of a U6:shRNA: EF-1α: Gfa2.2:Dlx2:WPRE:hGH.

FIG. 7 depicts establishment of rat astrocyte primary culture from 3 day post-natal Sprague-Dawley rat brains. Upper left panel presents an image of GFAP stained cells. Upper right panel presents an image of SOX9 stained cells. Lower left panel presents an image of DAPI stained cells. Lower right panel presents a merged image of GFAP, SOX9, and DAPI stained cells.

FIG. 8 depicts comparison of Dlx2 plasmid efficiency. Primary rat astrocyte cells are transfected with either the P44 (pEF-1α:Gfa681:Dlx2:WPRE:SV40), P60 (pEF-1α:Gfa681:Dlx2: shortened chimeric intron: WPRE:SV40), and P75 (CE:Gfa681:Dlx2:WPRE:SV40). Top panels show Dlx2 staining of cells, bottom panels show merged Dlx2 and DAPI staining of cells.

FIGS. 9A and 9B depicts quantitative analysis of AAV particle transduction into primary rate astrocytes. FIG. 9A presents the percentage transduction rate of AAV9-P12 (pGfa681:GFP) and AAV5-P7 (pEF-1α:GFP) at MOI of 5×10⁵ vg/cell, 2×10⁵ vg/cell, and 5×10⁴ vg/cell. FIG. 9B presents the percentage transduction rate of AAV9-P12 (pGfa681:GFP) in cells seeded at a series of densities of 2×10⁴ cell/well, 1.5×10⁴ cell/well, 1×10⁴ cell/well, and 5×10³ cell/well and infected with virus at a series of amounts of 2 μl, 1 μl, 0.5 μl, 0.25 μl, 0.125 μl of 1×10¹³ vg/ml virus in 100 μl of medium.

FIG. 10 depicts rat cortical astrocytes (RCAs) immunostained with an anti-Dlx2 antibody and DAPI (nuclear stain) 24 hours post transfection with NXL-P104 (CE-pGfa681-CGRI-Dlx2-bGHpA) or NXL-P105 (CE-pGfa681-CI-Dlx2-oPRE-bGHpA).

FIG. 11 depicts rat cortical astrocytes (RCAs) immunostained with an anti-Dlx2 antibody and DAPI (nuclear stain) 24 hours post transfection with NXL-P133 (EE-pGfa681-CGRI-Dlx2-oPRE-bGHpA), NXL-P137 (EE-pGfa681-CGRI-Dlx2-oPRE-bGHpA), or NXL-P131 (EE-pGfa681-CI-Dlx2-oPRE-bGHpA).

FIG. 12 depicts rat cortical astrocytes (RCAs) immunostained with an anti-Dlx2 antibody and DAPI (nuclear stain) 6 days post transduction with AAV9-P133 (CE-pGfa681-CGRI-Dlx2-oPRE-bGHpA).

### BRIEF DESCRIPTION OF SEQUENCES

A listing of nucleic acid sequences and amino acid sequences is provided in Table 1.

TABLE 1
Nucleic acid and amino acid sequences

| SEQ | Sequence | Sequence | |
|---|---|---|---|
| ID NO | Description | Type | Sequence |
| 1 | Upstream | Nucleic | TGCAGGCAGCTGCGCGCTCGCTCGCTCACTGAGGCCGCCCGGGC |
| | AAV2 ITR | acid | GTCGGGCGACCTTTGGTCGCCCGGCCTCAGTGAGCGAGCGAGCG |
| | | | CGCAGAGAGGGAGTGGCCAACTCCATCACTAGGGGTTCCT |
| 2 | Ef1a | Nucleic | TGCAAAGATGGATAAAGTTTTAAACAGAGAGGAATCTTTGCAGCTAAT |
| | enhancer | acid | GGACCTTCTAGGTCTTGAAAGGAGTGGGAATTGGCTCCGGTGCCCGTC |
| | | | AGTGGGCAGAGCGCACATCGCCCACAGTCCCCGAGAAGTTGGGGGGAG |
| | | | GGGTCGGCA |
| 3 | Gfa681 | Nucleic | CTAGTAACATATCCTGGTGTGGAGTAGGGGACGCTGCTCTGACA |
| | promoter | acid | GAGGCTCGGGGGCCTGAGCTGGCTCTGTGAGCTGGGGAGGAGGC |
| | | | AGACAGCCAGGCCTTGTCTGCAAGCAGACCTGGCAGCATTGGGC |
| | | | TGGCCGCCCCCCAGGGCCTCCTCTTCATGCCCAGTGAATGACTC |
| | | | ACCTTGGCACAGACACAATGTTCGGGGTGGGCACAGTGCCTGCT |
| | | | TCCCGCCGCACCCCAGCCCCCCTCAAATGCCTTCCGAGAAGCCC |
| | | | ATTGAGCAGGGGGCTTGCATTGCACCCCAGCCTGACAGCCTGGC |
| | | | ATCTTGGGATAAAAGCAGCACAGCCCCCTAGGGGCTGCCCTTGC |
| | | | TGTGTGGCGCCACCGGCGGTGGAGAACAAGGCTCTATTCAGCCT |
| | | | GTGCCCAGGAAAGGGGATCAGGGGATGCCCAGGCATGGACAGTG |
| | | | GGTGGCAGGGGGGGAGAGGAGGGCTGTCTGCTTCCCAGAAGTCC |
| | | | AAGGACACAAATGGGTGAGGGGAGAGCTCTCCCCATAGCTGGGC |
| | | | TGCGGCCCAACCCCACCCCCTCAGGCTATGCCAGGGGGTGTTGC |
| | | | CAGGGGCACCCGGGCATCGCCAGTCTAGCCCACTCCTTCATAAA |
| | | | GCCCTCGCATCCCAGGAGCGAGCAGAGCCAGAGCAGGTTGGAGA |
| | | | GGAGACGCATCACCTCCGCTGCTCGC |
| 4 | Gfa1.6 | Nucleic | CTGCAAGCAGACCTGGCAGCATTGGGCTGGCCGCCCCCCAGGGCCTCC |
| | promoter | acid | TCTTCATGCCCAGTGAATGACTCACCTTGGCACAGACACAATGTTCGG |
| | | | GGTGGGCACAGTGCCTGCTTCCCGCCGCACCCCAGCCCCCCTCAAATG |
| | | | CCTTCCGAGAAGCCCATTGAGTAGGGGGCTTGCATTGCACCCCAGCCT |
| | | | GACAGCCTGGCATCTTGGGATAAAAGCAGCACAGCCCCCTAGGGGCTG |
| | | | CCCTTGCTGTGTGGCGCCACCGGCGGTGGAGAACAAGGCTCTATTCAG |
| | | | CCTGTGCCCAGGAAAGGGGATCAGGGGATGCCCAGGCATGGACAGTGG |
| | | | GTGGCAGGGGGGGAGAGGAGGGCTGTCTGCTTCCCAGAAGTCCAAGGA |
| | | | CACAAATGGGTGAGGGGACTGGGCAGGGTTCTGACCCTGTGGGACCAG |
| | | | AGTGGAGGGCGTAGATGGACCTGAAGTCTCCAGGGACAACAGGGCCCA |
| | | | GGTCTCAGGCTCCTAGTTGGGCCCAGTGGCTCCAGCGTTTCCAAACCC |
| | | | ATCCATCCCCAGAGGTTCTTCCCATCTCTCCAGGCTGATGTGTGGGAA |
| | | | CTCGAGGAAATAAATCTCCAGTGGGAGACGGAGGGGTGGCCAGGGAAA |
| | | | CGGGGCGCTGCAGGAATAAAGACGAGCCAGCACAGCCAGCTCATGCGT |
| | | | AACGGCTTTGTGGAGCTGTCAAGGCCTGGTCTCTGGGAGAGAGGCACA |
| | | | GGGAGGCCAGACAAGGAAGGGGTGACCTGGAGGGACAGATCCAGGGGC |
| | | | TAAAGTCCTGATAAGGCAAGAGAGTGCCGGCCCCCTCTTGCCCTATCA |
| | | | GGACCTCCACTGCCACATAGAGGCCATGATTGACCCTTAGACAAAGGG |
| | | | CTGGTGTCCAATCCCAGCCCCCAGCCCCAGAACTCCAGGGAATGAATG |
| | | | GGCAGAGAGCAGGAATGTGGGACATCTGTGTTCAAGGGAAGGACTCCA |
| | | | GGAGTCTGCTGGGAATGAGGCCTAGTAGGAAATGAGGTGGCCCTTGAG |
| | | | GGTACAGAACAGGTTCATTCTTCGCCAAATTCCCAGCACCTTGCAGGC |
| | | | ACTTACAGCTGAGTGAGATAATGCCTGGGTTATGAAATCAAAAAGTTG |
| | | | GAAAGCAGGTCAGAGGTCATCTGGTACAGCCCTTCCTTCCCTTTTTTT |
| | | | TTTTTTTTTTTTGTGAGACAAGGTCTCTCTCTGTTGCCCAGGCTGGAG |
| | | | TGGCGCAAACACAGCTCACTGCAGCCTCAACCTACTGGGCTCAAGCAA |
| | | | TCCTCCAGCCTCAGCCTCCCAAAGTGCTGGGATTACAAGCATGAGCCA |
| | | | CCCCACTCAGCCCTTTCCTTCCTTTTTAATTGATGCATAATAATTGTA |
| | | | AGTATTCATCATGGTCCAACCAACCCTTTCTTGACCCACCTTCCTAGA |
| | | | GAGAGGGTCCTCTTGATTCAGCGGTCAGGGCCCCAGACCCATGGTCTG |
| | | | GCTCCAGGTACCACCTGCCTCATGCAGGAGTTGGCGTGCCCAGGAAGC |
| | | | TCTGCCTCTGGGCACAGTGACCTCAGTGGGGTGAGGGGAGCTCTCCCC |
| | | | ATAGCTGGGCTGCGGCCCAACCCCACCCCCTCAGGCTATGCCAGGGGG |
| | | | TGTTGCCAGGGGCACCCGGGCATCGCCAGTCTAGCCCACTCCTTCATA |
| | | | AAGCCCTCGCATCCCAGGAGCGAGCAGAGCCAGAG |
| 5 | Chimeric | Nucleic | GTAAGTATCAAGGTTACAAGACAGGTTTAAGGAGACCAATAGAAACTG |
| | Intron | acid | GGCTTGTCGAGACAGAGAAGACTCTTGCGTTTCTGATAGGCACCTATT |
| | | | GGTCTTACTGACATCCACTTTGCCTTTCTCTCCACAG |
| 6 | Human Dlx2 | Nucleic | ATGACTGGAGTCTTTGACAGTCTAGTGGCTGATATGCACTCGACCCAG |
| | | acid | ATCGCCGCCTCCAGCACGTACCACCAGCACCAGCAGCCCCCGAGCGGC |
| | | | GGCGGCGCCGGCCCGGGTGGCAACAGCAGCAGCAGCAGCAGCCTCCAC |
| | | | AAGCCCCAGGAGTCGCCCACCCTTCCGGTGTCCACCGCCACCGACAGC |
| | | | AGCTACTACACCAACCAGCAGCACCCGGCGGGCGGCGGCGGCGGCGGG |
| | | | GGCTCGCCCTACGCGCACATGGGTTCCTACCAGTACCAAGCCAGCGGC |
| | | | CTCAACAACGTCCCTTACTCCGCCAAGAGCAGCTATGACCTGGGCTAC |
| | | | ACCGCCGCCTACACCTCCTACGCTCCCTATGGAACCAGTTCGTCCCCA |
| | | | GCCAACAACGAGCCTGAGAAGGAGGACCTTGAGCCTGAAATTCGGATA |
| | | | GTGAACGGGAAGCCAAAGAAAGTCCGGAAACCCCGCACCATCTACTCC |
| | | | AGTTTCCAGCTGGCGGCTCTTCAGCGGCGTTTCCAAAAGACTCAATAC |
| | | | TTGGCCTTGCCGGAGCGAGCCGAGCTGGCGGCCTCTCTGGGCCTCACC |
| | | | CAGACTCAGGTCAAAATCTGGTTCCAGAACCGCCGGTCCAAGTTCAAG |
| | | | AAGATGTGGAAAAGTGGTGAGATCCCCTCGGAGCAGCACCCTGGGGCC |
| | | | AGCGCTTCTCCACCTTGTGCTTCGCCGCCAGTCTCAGCGCCGGCCTCC |
| | | | TGGGACTTTGGTGTGCCGCAGCGGATGGCGGGCGGCGGTGGTCCGGGC |
| | | | AGTGGCGGCAGCGGCGCCGGCAGCTCGGGCTCCAGCCCGAGCAGCGCG |
| | | | GCCTCGGCTTTTCTGGGCAACTACCCCTGGTACCACCAGACCTCGGGA |
| | | | TCCGCCTCACACCTGCAGGCCACGGCGCCGCTGCTGCACCCCACTCAG |
| | | | ACCCCGCAGCCGCATCACCACCACCACCATCACGGCGGCGGGGGCGCC |
| | | | CCGGTGAGCGCGGGGACGATTTTCTGA |
| 7 | WPRE | Nucleic | AATCAACCTCTGGATTACAAAATTTGTGAAAGATTGACTGGTATTCTT |
| | (Woodchuck | acid | AACTATGTTGCTCCTTTTACGCTATGTGGATACGCTGCTTTAATGCCT |
| | Hepatitis | | TTGTATCATGCTATTGCTTCCCGTATGGCTTTCATTTTCTCCTCCTTG |
| | Virus | | TATAAATCCTGGTTGCTGTCTCTTTATGAGGAGTTGTGGCCCGTTGTC |
| | Posttran- | | AGGCAACGTGGCGTGGTGTGCACTGTGTTTGCTGACGCAACCCCCACT |
| | scriptional | | GGTTGGGGCATTGCCACCACCTGTCAGCTCCTTTCCGGGACTTTCGCT |
| | Regulatory | | TTCCCCCTCCCTATTGCCACGGCGGAACTCATCGCCGCCTGCCTTGCC |
| | Element) | | CGCTGCTGGACAGGGGCTCGGCTGTTGGGCACTGACAATTCCGTGGTG |
| | | | TTGTCGGGGAAATCATCGTCCTTTCCTTGGCTGCTCGCCTGTGTTGCC |
| | | | ACCTGGATTCTGCGCGGGACGTCCTTCTGCTACGTCCCTTCGGCCCTC |
| | | | AATCCAGCGGACCTTCCTTCCCGCGGCCTGCTGCCGGCTCTGCGGCCT |
| | | | CTTCCGCGTCTTCGCCTTCGCCCTCAGACGAGTCGGATCTCCCTTTGG |
| | | | GCCGCCTCCCCGC |
| 8 | SV40 | Nucleic | CGATCCACCGGATCTAGATAACTGATCATAATCAGCCATACCACATTT |
| | poly(A) | acid | GTAGAGGTTTTACTTGCTTTAAAAAACCTCCCACACCTCCCCCTGAAC |
| | signal | | CTGAAACATAAAATGAATGCAATTGTTGTTGTTAACTTGTTTATTGCA |
| | | | GCTTATAATGGTTACAAATAAAGCAATAGCATCACAAATTTCACAAAT |
| | | | AAAGCATTTTTTTCACTGCATTCTAGTTGTGGTTTGTCCAAACTCATC |
| | | | AATGTATCTTA |
| 9 | Downstream | Nucleic | AGGAACCCCTAGTGATGGAGTTGGCCACTCCCTCTCTGCGCGCTCGCT |
| | AAV2 ITR | acid | CGCTCACTGAGGCCGGGCGACCAAAGGTCGCCCGACGCCCGGGCTTTG |
| | | | CCCGGGCGGCCTCAGTGAGCGAGCGAGCGCGCAGCTGCCTGCA |
| 10 | Human Dlx2 | Amino | MTGVFDSLVADMHSTQIAASSTYHQHQQPPSGGGAGPGGNSSSSSSLH |
| | | Acid | KPQESPTLPVSTATDSSYYTNQQHPAGGGGGGGSPYAHMGSYQYQASG |
| | | | LNNVPYSAKSSYDLGYTAAYTSYAPYGTSSSPANNEPEKEDLEPEIRI |
| | | | VNGKPKKVRKPRTIYSSFQLAALQRRFQKTQYLALPERAELAASLGLT |
| | | | QTQVKIWFQNRRSKFKKMWKSGEIPSEQHPGASASPPCASPPVSAPAS |
| | | | WDFGVPQRMAGGGGPGSGGSGAGSSGSSPSSAASAFLGNYPWYHQTSG |
| | | | SASHLQATAPLLHPTQTPQPHHHHHHHGGGGAPVSAGTIF |
| 11 | CMV | Nucleic | GACATTGATTATTGACTAGTTATTAATAGTAATCAATTACGGGGTCAT |
| | enhancer | Acid | TAGTTCATAGCCCATATATGGAGTTCCGCGTTACATAACTTACGGTAA |
| | (“CE”) | | ATGGCCCGCCTGGCTGACCGCCCAACGACCCCCGCCCATTGACGTCAA |
| | | | TAATGACGTATGTTCCCATAGTAACGCCAATAGGGACTTTCCATTGAC |
| | | | GTCAATGGGTGGACTATTTACGGTAAACTGCCCACTTGGCAGTACATC |
| | | | AAGTGTATCATATGCCAAGTACGCCCCCTATTGACGTCAATGACGGTA |
| | | | AATGGCCCGCCTGGCATTATGCCCAGTACATGACCTTATGGGACTTTC |
| | | | CTACTTGGCAGTACATCTACGTATTAGTCATCGCTATTACCATG |
| 12 | hGFA2.2 | Nucleic | CGCGTCCCACCTCCCTCTCTGTGCTGGGACTCACAGAGGGAGACCTCA |
| | promoter | Acid | GGAGGCAGTCTGTCCATCACATGTCCAAATGCAGAGCATACCCTGGGC |
| | | | TGGGCGCAGTGGCGCACAACTGTAATTCCAGCACTTTGGGAGGCTGAT |
| | | | GTGGAAGGATCACTTGAGCCCAGAAGTTCTAGACCAGCCTGGGCAACA |
| | | | TGGCAAGACCCTATCTCTACAAAAAAAGTTAAAAAATCAGCCACGTGT |
| | | | GGTGACACACACCTGTAGTCCCAGCTATTCAGGAGGCTGAGGTGAGGG |
| | | | GATCACTTAAGGCTGGGAGGTTGAGGCTGCAGTGAGTCGTGGTTGCGC |
| | | | CACTGCACTCCAGCCTGGGCAACAGTGAGACCCTGTCTCAAAAGACAA |
| | | | AAAAAAAAAAAAAAAAAAAAAGAACATATCCTGGTGTGGAGTAGGGGA |
| | | | CGCTGCTCTGACAGAGGCTCGGGGGCCTGAGCTGGCTCTGTGAGCTGG |
| | | | GGAGGAGGCAGACAGCCAGGCCTTGTCTGCAAGCAGACCTGGCAGCAT |
| | | | TGGGCTGGCCGCCCCCCAGGGCCTCCTCTTCATGCCCAGTGAATGACT |
| | | | CACCTTGGCACAGACACAATGTTCGGGGTGGGCACAGTGCCTGCTTCC |
| | | | CGCCGCACCCCAGCCCCCCTCAAATGCCTTCCGAGAAGCCCATTGAGC |
| | | | AGGGGGCTTGCATTGCACCCCAGCCTGACAGCCTGGCATCTTGGGATA |
| | | | AAAGCAGCACAGCCCCCTAGGGGCTGCCCTTGCTGTGTGGCGCCACCG |
| | | | GCGGTGGAGAACAAGGCTCTATTCAGCCTGTGCCCAGGAAAGGGGATC |
| | | | AGGGGATGCCCAGGCATGGACAGTGGGTGGCAGGGGGGGAGAGGAGGG |
| | | | CTGTCTGCTTCCCAGAAGTCCAAGGACACAAATGGGTGAGGGGACTGG |
| | | | GCAGGGTTCTGACCCTGTGGGACCAGAGTGGAGGGCGTAGATGGACCT |
| | | | GAAGTCTCCAGGGACAACAGGGCCCAGGTCTCAGGCTCCTAGTTGGGC |
| | | | CCAGTGGCTCCAGCGTTTCCAAACCCATCCATCCCCAGAGGTTCTTCC |
| | | | CATCTCTCCAGGCTGATGTGTGGGAACTCGAGGAAATAAATCTCCAGT |
| | | | GGGAGACGGAGGGGTGGCCAGGGAAACGGGGCGCTGCAGGAATAAAGA |
| | | | CGAGCCAGCACAGCCAGCTCATGTGTAACGGCTTTGTGGAGCTGTCAA |
| | | | GGCCTGGTCTCTGGGAGAGAGGCACAGGGAGGCCAGACAAGGAAGGGG |
| | | | TGACCTGGAGGGACAGATCCAGGGGCTAAAGTCCTGATAAGGCAAGAG |
| | | | AGTGCCGGCCCCCTCTTGCCCTATCAGGACCTCCACTGCCACATAGAG |
| | | | GCCATGATTGACCCTTAGACAAAGGGCTGGTGTCCAATCCCAGCCCCC |
| | | | AGCCCCAGAACTCCAGGGAATGAATGGGCAGAGAGCAGGAATGTGGGA |
| | | | CATCTGTGTTCAAGGGAAGGACTCCAGGAGTCTGCTGGGAATGAGGCC |
| | | | TAGTAGGAAATGAGGTGGCCCTTGAGGGTACAGAACAGGTTCATTCTT |
| | | | CGCCAAATTCCCAGCACCTTGCAGGCACTTACAGCTGAGTGAGATAAT |
| | | | GCCTGGGTTATGAAATCAAAAAGTTGGAAAGCAGGTCAGAGGTCATCT |
| | | | GGTACAGCCCTTCCTTCCCTTTTTTTTTTTTTTTTTTTGTGAGACAAG |
| | | | GTCTCTCTCTGTTGCCCAGGCTGGAGTGGCGCAAACACAGCTCACTGC |
| | | | AGCCTCAACCTACTGGGCTCAAGCAATCCTCCAGCCTCAGCCTCCCAA |
| | | | AGTGCTGGGATTACAAGCATGAGCCACCCCACTCAGCCCTTTCCTTCC |
| | | | TTTTTAATTGATGCATAATAATTGTAAGTATTCATCATGGTCCAACCA |
| | | | ACCCTTTCTTGACCCACCTTCCTAGAGAGAGGGTCCTCTTGCTTCAGC |
| | | | GGTCAGGGCCCCAGACCCATGGTCTGGCTCCAGGTACCACCTGCCTCA |
| | | | TGCAGGAGTTGGCGTGCCCAGGAAGCTCTGCCTCTGGGCACAGTGACC |
| | | | TCAGTGGGGTGAGGGGAGCTCTCCCCATAGCTGGGCTGCGGCCCAACC |
| | | | CCACCCCCTCAGGCTATGCCAGGGGGTGTTGCCAGGGGCACCCGGGCA |
| | | | TCGCCAGTCTAGCCCACTCCTTCATAAAGCCCTCGCATCCCAGGAGCG |
| | | | AGCAGAGCCAGAGCAGGTTGGAGAGGAGACGCATCACCTCCGCTGCTC |
| | | | GCCGGG |
| 13 | hGH poly(A) | Nucleic | GGGTGGCATCCCTGTGACCCCTCCCCAGTGCCTCTCCTGGCCCTGGAA |
| | signal | Acid | GTTGCCACTCCAGTGCCCACCAGCCTTGTCCTAATAAAATTAAGTTGC |
| | | | ATCATTTTGTCTGACTAGGTGTCCTTCTATAATATTATGGGGTGGAGG |
| | | | GGGGTGGTATGGAGCAAGGGGCAAGTTGGGAAGACAACCTGTAGGGCC |
| | | | TGCGGGGTCTATTGGGAACCAAGCTGGAGTGCAGTGGCACAATCTTGG |
| | | | CTCACTGCAATCTCCGCCTCCTGGGTTCAAGCGATTCTCCTGCCTCAG |
| | | | CCTCCCGAGTTGTTGGGATTCCAGGCATGCATGACCAGGCTCAGCTAA |
| | | | TTTTTGTTTTTTTGGTAGAGACGGGGTTTCACCATATTGGCCAGGCTG |
| | | | GTCTCCAACTCCTAATCTCAGGTGATCTACCCACCTTGGCCTCCCAAA |
| | | | TTGCTGGGATTACAGGCGTGAACCACTGCTCCCTTCCCTGTCCTT |
| 14 | U6 promoter | Nucleic | GTCCTTTCCACAAGATATATAAACCCAAGAAATCGAAATACTTTCAAG |
| | | acid | TTACGGTAAGCATATGATAGTCCATTTTAAAACATAATTTTAAAACTG |
| | | | CAAACTACCCAAGAAATTATTACTTTCTACGTCACGTATTTTGTACTA |
| | | | ATATCTTTGTGTTTACAGTCAAATTAATTCTAATTATCTCTCTAACAG |
| | | | CCTTGTATCGTATATGCAAATATGAAGGAATCATGGGAAATAGGCCCT |
| | | | C |
| 15 | Htt shRNA 1 | Nucleic | CCGGTGGTTCAGTTACGGGTTAATTCTCGAGAATTAACCCGTAACTGA |
| | | acid | ACCATTTTTG |
| 16 | Htt shRNA 2 | Nucleic | CCGGCAGTTACGGGTTAATTAATACCTGACCCATATTAATTAACCCGT |
| | | acid | AACTGCTTTTTG |
| 17 | Htt shRNA 3 | Nucleic | CCGGTGTTGCCGCAGCATCACTAATCTCGAGATTAGTGATGCTGCGGC |
| | | acid | AACATTTTG |
| 18 | oPRE | Nucleic | GAGCATCTTACCGCCATTTATACCCATATTTGTTCTGTTTTTCTTGAT |
| | (Optimized | Acid | TTGGGTATACATTTAAATGTTAATAAAACAAAATGGTGGGGCAATCAT |
| | Woodchuck | | TTACATTTTTAGGGATATGTAATTACTAGTTCAGGTGTATTGCCACAA |
| | Hepatitis | | GACAAACATGTTAAGAAACTTTCCCGTTATTTACGCTCTGTTCCTGTT |
| | Virus | | AATCAACCTCTGGATTACAAAATTTGTGAAAGATTGACTGATATTCTT |
| | Posttran- | | AACTATGTTGCTCCTTTTACGCTGTGTGGATATGCTGCTTTATAGCCT |
| | scriptional | | CTGTATCTAGCTATTGCTTCCCGTACGGCTTTCGTTTTCTCCTCCTTG |
| | Regulatory | | TATAAATCCTGGTTGCTGTCTCTTTTAGAGGAGTTGTGGCCCGTTGTC |
| | Element) | | CGTCAACGTGGCGTGGTGTGCTCTGTGTTTGCTGACGCAACCCCCACT |
| | | | GGCTGGGGCATTGCCACCACCTGTCAACTCCTTTCTGGGACTTTCGCT |
| | | | TTCCCCCTCCCGATCGCCACGGCAGAACTCATCGCCGCCTGCCTTGCC |
| | | | CGCTGCTGGACAGGGGCTAGGTTGCTGGGCACTGATAATTCCGTGGTG |
| | | | TTGTC |
| 19 | Chimeric | Nucleic | GGAGTCGCTGCGTTGCCTTCGCCCCGTGCCCCGCTCCGC |
| | intron of | Acid | GCCGCCTCGCGCCGCCCGCCCCGGCTCTGACTGACCGC |
| | rabbit beta- | | GTTACTCCCACAGGTGAGCGGGCGGGACGGCCCTTCTC |
| | globing and | | CCTCCGGGCTGTAATTAGCGCTTGGTTTAATGACGGCTC |
| | chicken beta | | GTTTCTTTTCTGTGGCTGCGTGAAAGCCTTAAAGGGCTC |
| | actin similar | | CGGGAGGGCCTTTGTGCGGGGGGGAGCGGCTCGGGGG |
| | in CAG | | GTGCGTGCGTGTGTGTGTGCGTGGGGAGCGCCGCGTGC |
| | promoter | | GGCCCGCGCTGCCCGGCGGCTGTGAGCGCTGCGGGCGC |
| | (CRGI) | | GGCGCGGGGCTTTGTGCGCTCCGCGTGTGCGCGAGGGG |
| | | | AGCGCGGGCCGGGGGCGGTGCCCCGCGGTGCGGGGGG |
| | | | GCTGCGAGGGGAACAAAGGCTGCGTGCGGGGTGTGTGC |
| | | | GTGGGGGGGTGAGCAGGGGGTGTGGGCGCGGCGGTCG |
| | | | GGCTGTAACCCCCCCCTGGCACCCCCCTCCCCGAGTTGC |
| | | | TGAGCACGGCCCGGCTTCGGGTGCGGGGCTCCGTGCGG |
| | | | GGCGTGGCGCGGGGCTCGCCGTGCCGGGCGGGGGGTGG |
| | | | CGGCAGGTGGGGGTGCCGGGCGGGGCGGGGCCGCCTC |
| | | | GGGCCGGGGAGGGCTCGGGGGAGGGGCGCGGCGGCCC |
| | | | CGGAGCGCCGGCGGCTGTCGAGGCGCGGCGAGCCGCA |
| | | | GCCATTGCCTTTTATGGTAATCGTGCGAGAGGGCGCAG |
| | | | GGACTTCCTTTGTCCCAAATCTGGCGGAGCCGAAATCTG |
| | | | GGAGGCGCCGCCGCACCCCCTCTAGCGGGCGCGGGCGA |
| | | | AGCGGTGCGGCGCCGGCAGGAAGGAAATGGGCGGGGA |
| | | | GGGCCTTCGTGCGTCGCCGCGCCGCCGTCCCCTTCTCCA |
| | | | TCTCCAGCCTCGGGGCTGCCGCAGGGGGACGGCTGCCT |
| | | | TCGGGGGGGACGGGGCAGGGCGGGGTTCGGCTTCTGGC |
| | | | GTGTGACCGGCGGCTTTAGAGCCTCTGCTAACCATGTTC |
| | | | ATGCCTTCTTCTTTTTCCTACAGCTCCTGGGCAACGTGC |
| | | | TGGTTGTTGTGCTGTCTCATCATTTTGGCAAAGAT |
| 20 | bGH poly | Nucleic | CTGTGCCTTCTAGTTGCCAGCCATCTGTTGTTTGCCCCTC |
| | (A) signal | acid | CCCCGTGCCTTCCTTGACCCTGGAAGGTGCCACTCCCAC |
| | (bGHpA) | | TGTCCTTTCCTAATAAAATGAGGAAATTGCATCGCATTG |
| | | | TCTGAGTAGGTGTCATTCTATTCTGGGGGGTGGGGTGG |
| | | | GGCAGGACAGCAAGGGGGAGGATTGGGAAGAGAATAG |
| | | | CAGGCATGCTGGGGA |
| 21 | p2A | Nucleic | GGAAGCGGAGCTACTAACTTCAGCCTGCTGAAGCAGGC |
| | | acid | TGGAGACGTGGAGGAGAACCCTGGACCT |

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

Any composition or vector provided herein is specifically envisioned for use with any method provided herein.

In an aspect, methods and compositions provided herein comprise a vector. As used herein, the term “vector” refers to a circular, double-stranded DNA molecule that is physically separate from chromosomal DNA. It should be noted that the term “vector” can be used interchangeably with the term “plasmid.”

In an aspect, a vector provided herein is a recombinant vector. As used herein, the term “recombinant vector” refers to a vector that comprises a recombinant nucleic acid. As used herein, a “recombinant nucleic acid” refers to a nucleic acid molecule formed by laboratory methods of genetic recombination, such as, without being limiting, molecular cloning. A recombinant vector can be formed by laboratory methods of genetic recombination, such as, without being limiting, molecular cloning. Also, without being limiting, one skilled in the art can create a recombinant vector de novo via synthesizing a plasmid by individual nucleotides, or by splicing together nucleic acid molecules from different pre-existing vectors.

Adeno-associated viruses (AAVs) are replication-defective, non-enveloped Dependoparvovirus viruses that infect humans and additional primate species. AAVs are not known to cause disease in any species, although they can cause mild immune responses. AAVs can infect dividing and quiescent cells. AAVs are stably integrate into the human genome at a specific site in chromosome 19 termed the AAVS1 locus (nucleotides 7774-11429 of GenBank Accession No. AC010327.8), although random integrations at other loci in the human genome are possible.

AAVs comprise a linear genome with a single-stranded DNA of about 4700 nucleotides in length. The genome of AAVs also includes a 145 nucleotide-long inverted terminal repeat (ITR) at each end of the genome. The ITRs flank two viral genes rep (for replication, encoding non-structural proteins) and cap (for capsid, encoding structural proteins). The ITRs contain all of the cis-acting elements need for genome rescue, replication, and packaging of the AAV.

When used in gene therapy approaches, the rep and cap genes of the AAV genome sequence are removed and replaced with DNA of interest positioned between two AAV ITRs. As used herein, an “AAV vector construct” refers to a DNA molecule comprising a desired sequence inserted between two AAV ITR sequences. As used herein, an “AAV vector” refers to an AAV packaged with a DNA vector construct.

As used herein, the term “AAV vector serotype” mainly refers to a variation within the capsid proteins of an AAV vector.

As used herein “vg” refers to a viral genome in the context of AAV titer determination.

In an aspect, an AAV vector is selected from the group consisting of AAV vector serotype 1, AAV vector serotype 2, AAV vector serotype 3, AAV vector serotype 4, AAV vector serotype 5, AAV vector serotype 6, AAV vector serotype 7, AAV vector serotype 8, AAV vector serotype 9, AAV vector serotype 10, AAV vector serotype 11, and AAV vector serotype 12. In one aspect, an AAV vector is selected from the group consisting AAV serotype 2, AAV serotype 5, and AAV serotype 9. In one aspect, an AAV vector is AAV serotype 1. In one aspect, an AAV vector is AAV serotype 2. In one aspect, an AAV vector is AAV serotype 3. In one aspect, an AAV vector is AAV serotype 4. In one aspect, an AAV vector is AAV serotype 5. In one aspect, an AAV vector is AAV serotype 6. In one aspect, an AAV vector is AAV serotype 7. In one aspect, an AAV vector is AAV serotype 8. In one aspect, an AAV vector is AAV serotype 9. In one aspect, an AAV vector is AAV serotype 10. In one aspect, an AAV vector is AAV serotype 11. In one aspect, an AAV vector is AAV serotype 12.

In an aspect, an AAV vector ITR is selected from the group consisting of an AAV serotype 1 ITR, an AAV serotype 2 ITR, an AAV serotype 3 ITR, an AAV serotype 4 ITR, an AAV serotype 5 ITR, an AAV serotype 6 ITR, an AAV serotype 7 ITR, an AAV serotype 8 ITR, an AAV serotype 9 ITR, an AAV serotype 10 ITR, an AAV serotype 11 ITR, and an AAV serotype 12 ITR. In one aspect, an AAV vector ITR is an AAV serotype 1 ITR. In one aspect, an AAV vector ITR is an AAV serotype 2 ITR. In one aspect, an AAV vector ITR is an AAV serotype 3 ITR. In one aspect, an AAV vector ITR is an AAV serotype 4 ITR. In one aspect, an AAV vector ITR is an AAV serotype 5 ITR. In one aspect, an AAV vector ITR is an AAV serotype 6 ITR. In one aspect, an AAV vector ITR is an AAV serotype 7 ITR. In one aspect, an AAV vector ITR is an AAV serotype 8 ITR. In one aspect, an AAV vector ITR is an AAV serotype 9 ITR. In one aspect, an AAV vector ITR is an AAV serotype 10 ITR. In one aspect, an AAV vector ITR is an AAV serotype 11 ITR. In one aspect, an AAV vector ITR is an AAV serotype 12 ITR.

In an aspect, at least one AAV vector ITR nucleic acid sequence is selected from the group consisting of SEQ ID NO: 1 and 9. In one aspect, at least one AAV vector ITR nucleic acid sequence is SEQ ID NO: 1. In one aspect, at least one AAV vector ITR nucleic acid sequence is SEQ ID NO: 9.

In an aspect, an AAV ITR nucleic acid sequence comprises a sequence at least 70% identical to SEQ ID NO: 1, or the complement thereof. In one aspect, an AAV ITR nucleic acid sequence comprises a sequence at least 75% identical to SEQ ID NO: 1, or the complement thereof. In one aspect, an AAV ITR nucleic acid sequence comprises a sequence at least 80% identical to SEQ ID NO: 1, or the complement thereof. In one aspect, an AAV ITR nucleic acid sequence comprises a sequence at least 85% identical to SEQ ID NO: 1, or the complement thereof. In one aspect, an AAV ITR nucleic acid sequence comprises a sequence at least 90% identical to SEQ ID NO: 1, or the complement thereof. In one aspect, an AAV ITR nucleic acid sequence comprises a sequence at least 91% identical to SEQ ID NO: 1, or the complement thereof. In one aspect, an AAV ITR nucleic acid sequence comprises a sequence at least 92% identical to SEQ ID NO: 1, or the complement thereof. In one aspect, an AAV ITR nucleic acid sequence comprises a sequence at least 93% identical to SEQ ID NO: 1, or the complement thereof. In one aspect, an AAV ITR nucleic acid sequence comprises a sequence at least 94% identical to SEQ ID NO: 1, or the complement thereof. In one aspect, an AAV ITR nucleic acid sequence comprises a sequence at least 95% identical to SEQ ID NO: 1, or the complement thereof. In one aspect, an AAV ITR nucleic acid sequence comprises a sequence at least 96% identical to SEQ ID NO: 1, or the complement thereof. In one aspect, an AAV ITR nucleic acid sequence comprises a sequence at least 97% identical to SEQ ID NO: 1, or the complement thereof. In one aspect, an AAV ITR nucleic acid sequence comprises a sequence at least 98% identical to SEQ ID NO: 1, or the complement thereof. In one aspect, an AAV ITR nucleic acid sequence comprises a sequence at least 99% identical to SEQ ID NO: 1, or the complement thereof. In one aspect, an AAV ITR nucleic acid sequence comprises a sequence at least 99.5% identical to SEQ ID NO: 1, or the complement thereof. In one aspect, an AAV ITR nucleic acid sequence comprises a sequence at least 99.8% identical to SEQ ID NO: 1, or the complement thereof. In one aspect, an AAV ITR nucleic acid sequence comprises a sequence at least 99.9% identical to SEQ ID NO: 1, or the complement thereof. In one aspect, an AAV ITR nucleic acid sequence comprises a sequence 100% identical to SEQ ID NO: 1, or the complement thereof.

In an aspect, an AAV ITR nucleic acid sequence comprises a sequence at least 70% identical to SEQ ID NO: 9, or the complement thereof. In one aspect, an AAV ITR nucleic acid sequence comprises a sequence at least 75% identical to SEQ ID NO: 9, or the complement thereof. In one aspect, an AAV ITR nucleic acid sequence comprises a sequence at least 80% identical to SEQ ID NO: 9, or the complement thereof. In one aspect, an AAV ITR nucleic acid sequence comprises a sequence at least 85% identical to SEQ ID NO: 9, or the complement thereof. In one aspect, an AAV ITR nucleic acid sequence comprises a sequence at least 90% identical to SEQ ID NO: 9, or the complement thereof. In one aspect, an AAV ITR nucleic acid sequence comprises a sequence at least 91% identical to SEQ ID NO: 9, or the complement thereof. In one aspect, an AAV ITR nucleic acid sequence comprises a sequence at least 92% identical to SEQ ID NO: 9, or the complement thereof. In one aspect, an AAV ITR nucleic acid sequence comprises a sequence at least 93% identical to SEQ ID NO: 9, or the complement thereof. In one aspect, an AAV ITR nucleic acid sequence comprises a sequence at least 94% identical to SEQ ID NO: 9, or the complement thereof. In one aspect, an AAV ITR nucleic acid sequence comprises a sequence at least 95% identical to SEQ ID NO: 9, or the complement thereof. In one aspect, an AAV ITR nucleic acid sequence comprises a sequence at least 96% identical to SEQ ID NO: 9, or the complement thereof. In one aspect, an AAV ITR nucleic acid sequence comprises a sequence at least 97% identical to SEQ ID NO: 9, or the complement thereof. In one aspect, an AAV ITR nucleic acid sequence comprises a sequence at least 98% identical to SEQ ID NO: 9, or the complement thereof. In one aspect, an AAV ITR nucleic acid sequence comprises a sequence at least 99% identical to SEQ ID NO: 9, or the complement thereof. In one aspect, an AAV ITR nucleic acid sequence comprises a sequence at least 99.5% identical to SEQ ID NO: 9, or the complement thereof. In one aspect, an AAV ITR nucleic acid sequence comprises a sequence at least 99.8% identical to SEQ ID NO: 9, or the complement thereof. In one aspect, an AAV ITR nucleic acid sequence comprises a sequence at least 99.9% identical to SEQ ID NO: 9, or the complement thereof. In one aspect, an AAV ITR nucleic acid sequence comprises a sequence 100% identical to SEQ ID NO: 9, or the complement thereof.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide,” “nucleic acid sequence,” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In one aspect, a nucleic acid molecule provided herein is an RNA molecule. In one aspect, a nucleic acid molecule provided herein is single-stranded. In one aspect, a nucleic acid molecule provided herein is double-stranded. A nucleic acid molecule can encode a polypeptide or a small RNA.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell. As used herein, a “coding sequence” refers to a nucleic acid sequence that encodes a protein.

As used herein, the term “CpG site” or “CG site” refers to a region of DNA sequence where a cytosine and guanine is separated by only one phosphate group.

As used herein, the term “CpG island” of “CG island” refers to CpG sites that occur with a high frequency.

As used herein, the term “codon” refers to a sequence of three nucleotides.

As used herein, the term “codon optimized” refers to a code that is modified for enhanced expression in a host cell of interest by replacing at least one codon of a sequence with codons that are more frequently or most frequently used in the genes of the host cell while maintaining the original amino acid sequence.

As used herein, the term “enhancer” refers to a region of DNA sequence that operates to initiate, assist, affect, cause, and/or promote the transcription and expression of the associated transcribable DNA sequence or coding sequence, at least in certain tissue(s), developmental stage(s) and/or condition(s). In an aspect, an enhancer is a cis enhancer. In one aspect, an enhancer is a trans enhancer.

As used herein “CE” refers to a cytomegalovirus (CMV) promoter enhancer sequence.

As used here “EE” refers to an Ef1 alpha promoter enhancer sequence.

Enhancer sequences can be identified by utilizing genomic techniques well known in the art. Non-limiting examples include use of a reporter gene and next-generation sequencing methods such as chromatin immunoprecipitation sequencing (ChIP-seq), DNase I hypersensitivity sequencing (DNase-seq), micrococcal nuclease sequencing (MNase-seq), formaldehyde-assisted isolation of regulatory elements sequencing (FAIRE-seq), and assay for transposase accessible chromatin sequencing (ATAC-seq).

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

As used herein “p2A” or “P2A” refer to a 2A self-cleavage peptide sequence from porcine teschovirus-1. In an aspect, a linker may comprise a P2A sequence.

As used herein, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

As used herein, an “intron” refers to a nucleotide sequence that is removed by RNA splicing as a messenger RNA (mRNA) matures from a mRNA precursor.

As used herein, “mRNA” or “messenger RNA” refers to a single stranded RNA that corresponds to the genetic sequence of a gene.

Expression of mRNA can be measured using any suitable method known in the art. Non-limiting examples of measuring mRNA expression include quantitative reverse transcriptase polymerase chain reaction (qRT-PCR), RNA blot (e.g., a Northern blot), and RNA sequencing. Differences in expression can be described as an absolute quantification or a relative quantification. See, for example, Livak and Schmittgen, Methods, 25:402-408 (2001).

As used herein, “genome editing” or “gene editing” refers to targeted mutagenesis, insertion, deletion, inversion, substitution, or translocation of a nucleotide sequence of interest in a genome using a targeted editing technique. A nucleotide sequence of interest can be of any length, for example, at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides. Non-limiting examples of gene editing techniques are small interference RNA (siRNA) technology, small hairpin RNA (shRNA) technology, microRNA (miRNA) technology, antisense oligonucleotides (ASO) technology, or CRISPR/CAS technology.

As used herein, a “ASO” or “antisense oligonucleotide” is a small, single stranded nucleic acid that bind to their target RNA sequence inside the cells and silence genes.

As used herein, a “coding region,” a “gene region,” or a “gene” refers to a polynucleotide that can produce a functional unit. Non-limiting examples include a protein, or a non-coding RNA molecule. A “coding region,” “gene,” or “gene region” can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof.

In an aspect, gene editing targets mutant Huntingtin (Htt) aggregates. In one aspect gene editing is by non-coding RNA molecules. Non-limiting examples of a non-coding RNA molecule include a microRNA (miRNA), a miRNA precursor (pre-miRNA), a small interfering RNA (siRNA), a small RNA (18-26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single-guide RNA (sgRNA). In one aspect, a shRNA targets a Htt gene. In one aspect, a siRNA targets a Htt gene. In one aspect, an ASO targets a Htt gene. In one aspect, miRNA targets a Htt gene. In one aspect, a gRNA targets a Htt gene. In one aspect, a pre-miRNA targets a Htt gene. In one aspect, a small RNA targets a Htt gene. In one aspect, a he-siRNA targets a Htt gene. In one aspect, a piRNA targets a Htt gene. In one aspect, a hairpin dsRNA targets a Htt gene. In one aspect, a to-siRNA targets a Htt gene. In one aspect, a nat-siRNA targets a Htt gene. In one aspect, a crRNA targets a Htt gene. In one aspect, a tracrRNA targets a Htt gene. In one aspect, a sgRNA targets a Htt gene. In one aspect, a shRNA comprises a nucleic acid sequence selected from the group consisting of SEQ ID NOs: 15 to 17. In one aspect, a shRNA comprises a nucleic acid sequence SEQ ID NO: 15. In one aspect, a shRNA comprises a nucleic acid sequence SEQ ID NO: 16. In one aspect, a shRNA comprises a nucleic acid sequence SEQ ID NO: 17.

As used herein a “donor molecule” or “donor sequence” is defined as a nucleic acid sequence that has been selected for site directed, targeted insertion into a genome. In an aspect, a donor molecule comprises a “donor sequence.” In one aspect, a targeted editing technique provided herein comprises the use of one or more, two or more, three or more, four or more, or five or more donor molecules or donor sequences. A donor molecule or donor sequence provided herein can be of any length. For example, a donor molecule or donor sequence provided herein is between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000 or between 20 and 10,000 nucleotides in length.

As used here “HTT” refers to an Htt specific guide RNA (gRNA) and/or a donor sequence.

Site-specific nucleases provided herein can be used as part of a targeted editing technique. Non-limiting examples of site-specific nucleases include meganucleases, zinc finger nucleases (ZFNs), transcription activator-like effector nucleases (TALENs), RNA-guided nucleases (e.g., Cas9 and Cpf1), a recombinase (without being limiting, for example, a serine recombinase attached to a DNA recognition motif, a tyrosine recombinase attached to a DNA recognition motif), a transposase (without being limiting, for example, a DNA transposase attached to a DNA binding domain), or any combination thereof.

Site-specific nucleases, such as meganucleases, ZFNs, TALENs, Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof), Cas9 nucleases (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof), induce a double-strand DNA break at the target site of a genomic sequence that is then repaired by the natural processes. Sequence modifications then occur at the cleaved sites, which can include inversions, deletions, or insertions that result in gene disruption or integration of nucleic acid sequences. In an aspect, an RNA-guided nuclease provided herein is selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof.

In an aspect, a targeted editing technique described herein comprises the use of a RNA-guided nuclease.

While not being limited by any particular scientific theory, CRISPR/CAS nucleases are part of the adaptive immune system of bacteria and archaea, protecting them against invading nucleic acids such as viruses by cleaving target DNA in a sequence-dependent manner. The immunity is acquired by the integration of short fragments of the invading DNA, known as spacers, between ˜20 nucleotide long CRISPR repeats at the proximal end of a CRISPR locus (a CRISPR array). A well described Cas protein is the Cas9 nuclease (also known as Csn1), which is part of the Class 2, type II CRISPR/Cas system in Streptococcus pyogenes. See Makarova et al. Nature Reviews Microbiology (2015) doi: 10.1038/nrmicro3569. Cas9 comprises an RuvC-like nuclease domain at its amino terminus and an HNH-like nuclease domain positioned in the middle of the protein. Cas9 proteins also contain a PAM-interacting (PI) domain, a recognition lobe (REC), and a BH domain. The Cpf1 nuclease, another type II system, acts in a similar manner to Cas9, but Cpf1 does not require a tracrRNA. See Cong et al. Science (2013) 339: 819-823; Zetsche et al., Cell (2015) doi: 10.1016/j.ce11.2015.09.038; U. S. Patent Publication No. 2014/0068797; U.S. Patent Publication No. 2014/0273235; U. S. Patent Publication No. 2015/0067922; U. S. Pat. Nos. 8,697,359; 8,771,945; 8,795,965; 8,865,406; 8,871,445; 8,889,356; 8,889,418; 8,895,308; and 8,906,616, each of which is herein incorporated by reference in its entirety.

In an aspect, this disclosure provides, and includes, a composition comprising an adeno-associated virus (AAV) vector where the AAV vector comprises a Cas9 nuclease gene, an Htt specific gRNA, and a donor sequence. In an aspect, this disclosure provides, and includes, a composition comprising an adeno-associated virus (AAV) vector where the AAV vector comprises a Cas9 nuclease gene, an Htt specific gRNA, a donor sequence, and a Dlx2 gene sequence. In an aspect, this disclosure provides, and includes, a composition comprising an adeno-associated virus (AAV) vector where the AAV vector comprises a Cas9 nuclease gene, an Htt specific shRNA, and a donor sequence. In an aspect, this disclosure provides, and includes, a composition comprising an adeno-associated virus (AAV) vector where the AAV vector comprises a Cas9 nuclease gene, an Htt specific shRNA, a donor sequence, and a Dlx2 gene sequence.

As used herein, the term “glial” or “glial cell” refers to a non-neuronal cell in the CNS or the PNS. In an aspect, at least one glial cell is selected from the group consisting of at least one oligodendrocyte, at least one astrocyte, at least one NG2 cell, at least one ependymal cell, and at least one microglia. In one aspect, at least one glial cell is at least one oligodendrocyte. In one aspect, at least one glial cell is at least one NG2 cell. In one aspect, at least one glial cell is at least one ependymal cell. In one aspect, at least one glial cell is at least one microglia. In one aspect, at least one glial cell is at least one reactive astrocyte. In one aspect, at least one astrocyte is at least one reactive astrocyte.

As used herein, the term “astrocyte” refers to a glial cell that is an important component of the brain. An astrocyte is involved in supporting neuronal functions such as synapse formation and plasticity, potassium buffering, nutrient supply, the secretion and absorption of neural or glial transmitters, and maintenance of the blood-brain barrier. As used herein, the term “reactive astrocytes” refers to an abnormal status of astrocytes after injury or disease.

As used herein, the term “NG2 cell” or “polydendrocyte” refers to a glial cell that expresses chondroitin sulfate proteoglycan (CSPG4) and the alpha receptor for platelet-derived growth factor (PDGFRA).

As used herein, the term “neuron” or “neuronal cell” refers to an electrically excitable cell that communicates with other neurons via synapses. In an aspect, a neuron is selected from the group consisting of an unipolar neuron, a bipolar neuron, a pseudounipolar neuron, and a multipolar neuron. In one aspect, a neuron is an unipolar neuron. In one aspect, a neuron is a bipolar neuron. In one aspect, a neuron is apseudounipolar neuron. In one aspect, a neuron is a bipolar neuron. In one aspect, a neuron is selected from the group consisting of a sensory neuron, a motor neuron, and an interneuron. In one aspect, a neuron is a sensory neuron. In one aspect, a neuron is a motor neuron. In one aspect, a neuron is an interneuron.

As used herein, the term “functional neuron” refers to a neuron that can perform biological process. Without being limiting, examples of biological processes include processing and transmission of information and communication via chemical and electrical synapses.

As used herein, the term “glutamatergic neurons” refers to a subclass of neurons that produce glutamate and establish excitatory synapses. As used herein, the term “excitatory synapse” refers to a synapse in which an action potential in a presynaptic neuron increases the probability of an action potential occurring in a postsynaptic cell. As used herein, the term “action potential” or “nerve impulse” refers to an electrical impulse across the membrane of an axon. As used herein, the term “axon” or “nerve fiber” refers to a neuron that conducts action potentials. As used herein, the term “GABAergic neurons” refers to a subset of neurons that produce GABA and establish inhibitory synapses. As used herein, the term “GABA” or “gamma-Aminobutyric acid” refers to a compound that opens ion channels to allow the flow of negatively charged chloride ions into the cell or positively charged potassium ions out of the cell. As used herein, the term “inhibitory synapse” refers to a synapse that moves the membrane potential of a postsynaptic neuron away from the threshold for generating action potentials. As used herein, the term “dopaminergic neuron” refers to a subset of neurons that produce dopamine. As used herein, the term “dopamine” refers to a type of neurotransmitter. As used herein, the term “neurotransmitter” refers to a class of endogenous chemicals that activate neurotransmissions. As used herein, the term “neurotransmission” refers to a process where neurotransmitters are released by the axon terminal of a neuron. As used herein, the term “acetyl cholinergic neuron” or “cholinergic neuron” refers to a subset of neurons that secrete acetylcholine. As used herein, the term “acetylcholine” refers to a type of neurotransmitter. As used herein, the term “seratonergic neuron” refers to a subset of neurons that synthesizes serotonin. As used herein, the term “serotonin” refers to a type of neurotransmitter. As used herein, a “epinephrinergic neuron” refers to a neuron that release epinephrine as the neurotransmitter. As used herein, the term “motor neuron” refers to a subset of neurons where the cell body is located in the motor cortex, brainstem, or the spinal cord and the axon projects to the spinal cord or outside the spinal cord and directly or indirectly controls muscles and glands. As used herein, the term peptidergic neuron refers to a subset of neurons that utilize small peptide molecules as their neurotransmitter.

In an aspect, a neuron is a functional neuron. In one aspect, a functional neuron is selected from the group consisting of glutamatergic neurons, GABAergic neurons, dopaminergic neurons, cholinergic neurons, seratonergic neurons, epinephrinergic neurons, motor neurons, and peptidergic neurons. In one aspect, a functional neuron is a glutamatergic neuron. In one aspect, a functional neuron is a GABAergic neuron. In one aspect, a functional neuron is a dopaminergic neuron. In one aspect, a functional neuron is a cholinergic neuron. In one aspect, a functional neuron is a seratonergic neuron. In one aspect, a functional neuron is an epinephrinergic neuron. In one aspect, a functional neuron is a motor neuron. In one aspect, a functional neuron is a peptidergic neuron.

As used herein, the term “converting” or “converted” refers to a cell type changing its physical morphology and/or biological function into a different physical morphology and/or different biological function. In an aspect, this disclosure provides the conversion of at least one glial cell into at least one neuron. In one aspect, conversion of at least one glial cell to at least one neuron occurs in the CNS or PNS. In one aspect, conversion of at least one glial cell to at least one neuron occurs in the CNS. In one aspect, conversion of at least one glial cell to at least one neuron occurs in the PNS.

In an aspect, this disclosure provides, and includes, an adeno-associated virus (AAV) vector comprising a human distal-less homeobox 2 (hDlx2) sequence comprising the nucleic acid sequence of SEQ ID NO: 6, where the hDlx2 sequence is operably linked to regulatory elements comprising: (a) a glial fibrillary acid protein (GFAP) promoter comprising a nucleic acid sequence selected from the group consisting of SEQ ID NOs: 3, 4, and 12; (b) an enhancer from a human elongation factor-1 alpha (EF1-α) promoter comprising the nucleic acid sequence of SEQ ID NO: 2 or cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal sequence comprising the nucleic acid sequence of SEQ ID NO: 8, a hGH polyadenylation signal comprising the nucleic acid sequence of SEQ ID NO: 13 or a bGH polyadenylation signal comprising the nucleic acid sequence of SEQ ID NO: 20.

In an aspect, this disclosure provides an adeno-associated virus (AAV) vector comprising a nucleic acid coding sequence encoding a human distal-less homeobox 2 (hDlx2) protein comprising the amino acid sequence if SEQ ID NO: 10, where the coding sequence is operably linked to regulatory elements comprising: (a) a glial fibrillary acid protein (GFAP) promoter comprising a nucleic acid sequence selected from the group consisting of SEQ ID NOs: 3, 4, and 12; (b) an enhancer from a human elongation factor-1 alpha (EF-1 alpha) promoter comprising the nucleic acid sequence of SEQ ID NO: 2 or cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; a woodchuck hepatitis virus posttranscriptional regulatory element (WPRE) comprising the nucleic acid sequence selected from the group consisting of SEQ ID NOs: 7 and 18; and a SV40 polyadenylation signal with a nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, an adeno-associated virus (AAV) vector comprising a distal-less homeobox 2 (Dlx2) nucleic acid coding sequence encoding a Dlx2 protein, where the coding sequence is operably linked to regulatory elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal sequence.

In an aspect, this disclosure provides, and includes, a composition comprising an adeno-associated virus (AAV) vector for converting glial cells to functional neurons in a human, where the AAV vector comprises a human distal-less homeobox 2 (hDlx2) sequence having a nucleic acid sequence of SEQ ID NO: 6, and where the sequence is operably linked to regulatory elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, or a hGH polyadenylation signal comprising the nucleic acid sequence of SEQ ID NO: 13, or a bGH polyadenylation signal comprising the nucleic acid sequence of SEQ ID NO: 20.

In an aspect, this disclosure provides, and includes, a composition comprising an adeno-associated virus (AAV) vector for converting glial cells to functional neurons in a human, where the AAV vector comprises a nucleic acid coding sequence encoding a human distal-less homeobox 2 (hDlx2) protein comprising the amino acid sequence of SEQ ID NO: 10, and where the coding sequence is operably linked to regulatory elements comprising: (a) a human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising the nucleic acid sequence of SEQ ID NO: 13 or a bGH polyadenylation signal comprising the nucleic acid sequence of SEQ ID NO: 20.

In an aspect, this disclosure provides, and includes, a composition comprising an adeno-associated virus (AAV) vector for the treatment of a subject in need thereof, where the AAV vector comprises a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal.

In an aspect, an AAV vector comprises a nucleic acid sequence encoding an AAV protein. In one aspect, an AAV vector comprises a nucleic acid sequence encoding a viral protein. Non-limiting examples of AAV proteins and viral proteins include rep and cap proteins. Distal-less homeobox 2 (Dlx2: also referred to as TES1) is a member of the Dlx gene family and is a homeobox containing gene that plays a role in forebrain and craniofacial development.

In an aspect, a Dlx2 sequence is a human Dlx2 (hDlx2) sequence. In one aspect, a Dlx2 sequence is selected from the group consisting of a chimpanzee Dlx2 sequence, a bonobo Dlx2 sequence, an orangutan Dlx2 sequence, a gorilla Dlx2 sequence, a macaque Dlx2 sequence, a marmoset Dlx2 sequence, a capuchin Dlx2 sequence, a baboon Dlx2 sequence, a gibbon Dlx2 sequence, and a lemur Dlx2 sequence. In one aspect, a Dlx2 sequence is a chimpanzee Dlx2 sequence. In one aspect, a Dlx2 sequence is a bonobo Dlx2 sequence. In one aspect, a Dlx2 sequence is an orangutan Dlx2 sequence. In one aspect, a Dlx2 sequence is a gorilla Dlx2 sequence. In one aspect, a Dlx2 sequence is a macaque Dlx2 sequence. In one aspect, a Dlx2 sequence is a marmoset Dlx2 sequence. In one aspect, a Dlx2 sequence is a capuchin Dlx2 sequence. In one aspect, a Dlx2 sequence is a baboon Dlx2 sequence. In one aspect, a Dlx2 sequence is a gibbon Dlx2 sequence. In one aspect, a Dlx2 sequence is a lemur Dlx2 sequence.

In an aspect, a Dlx2 nucleic acid sequence comprises a sequence at least 70% identical to SEQ ID NO: 6, or the complement thereof. In one aspect, a Dlx2 nucleic acid sequence comprises a sequence at least 75% identical to SEQ ID NO: 6, or the complement thereof. In one aspect, a Dlx2 nucleic acid sequence comprises a sequence at least 80% identical to SEQ ID NO: 6, or the complement thereof. In one aspect, a Dlx2 nucleic acid sequence comprises a sequence at least 85% identical to SEQ ID NO: 6, or the complement thereof. In one aspect, a Dlx2 nucleic acid sequence comprises a sequence at least 90% identical to SEQ ID NO: 6, or the complement thereof. In one aspect, a Dlx2 nucleic acid sequence comprises a sequence at least 91% identical to SEQ ID NO: 6, or the complement thereof. In one aspect, a Dlx2 nucleic acid sequence comprises a sequence at least 92% identical to SEQ ID NO: 6, or the complement thereof. In one aspect, a Dlx2 nucleic acid sequence comprises a sequence at least 93% identical to SEQ ID NO: 6, or the complement thereof. In one aspect, a Dlx2 nucleic acid sequence comprises a sequence at least 94% identical to SEQ ID NO: 6, or the complement thereof. In one aspect, a Dlx2 nucleic acid sequence comprises a sequence at least 95% identical to SEQ ID NO: 6, or the complement thereof. In one aspect, a Dlx2 nucleic acid sequence comprises a sequence at least 96% identical to SEQ ID NO: 6, or the complement thereof. In one aspect, a Dlx2 nucleic acid sequence comprises a sequence at least 97% identical to SEQ ID NO: 6, or the complement thereof. In one aspect, a Dlx2 nucleic acid sequence comprises a sequence at least 98% identical to SEQ ID NO: 6, or the complement thereof. In one aspect, a Dlx2 nucleic acid sequence comprises a sequence at least 99% identical to SEQ ID NO: 6, or the complement thereof. In one aspect, a Dlx2 nucleic acid sequence comprises a sequence at least 99.5% identical to SEQ ID NO: 6, or the complement thereof. In one aspect, a Dlx2 nucleic acid sequence comprises a sequence at least 99.8% identical to SEQ ID NO: 6, or the complement thereof. In one aspect, a Dlx2 nucleic acid sequence comprises a sequence at least 99.9% identical to SEQ ID NO: 6, or the complement thereof. In one aspect, a Dlx2 nucleic acid sequence comprises a sequence 100% identical to SEQ ID NO: 6, or the complement thereof.

In an aspect, a nucleic acid coding sequence encodes a Dlx2 protein comprising an amino acid sequence at least 70% identical or similar to SEQ ID NO: 10. In one aspect, a nucleic acid coding sequence encodes a Dlx2 protein comprising an amino acid sequence at least 75% identical or similar to SEQ ID NO: 10. In one aspect, a nucleic acid coding sequence encodes a Dlx2 protein comprising an amino acid sequence at least 80% identical or similar to SEQ ID NO: 10. In one aspect, a nucleic acid coding sequence encodes a Dlx2 protein comprising an amino acid sequence at least 85% identical or similar to SEQ ID NO: 10. In one aspect, a nucleic acid coding sequence encodes a Dlx2 protein comprising an amino acid sequence at least 90% identical or similar to SEQ ID NO: 10. In one aspect, a nucleic acid coding sequence encodes a Dlx2 protein comprising an amino acid sequence at least 91% identical or similar to SEQ ID NO: 10. In one aspect, a nucleic acid coding sequence encodes a Dlx2 protein comprising an amino acid sequence at least 92% identical or similar to SEQ ID NO: 10. In one aspect, a nucleic acid coding sequence encodes a Dlx2 protein comprising an amino acid sequence at least 93% identical or similar to SEQ ID NO: 10. In one aspect, a nucleic acid coding sequence encodes a Dlx2 protein comprising an amino acid sequence at least 94% identical or similar to SEQ ID NO: 10. In one aspect, a nucleic acid coding sequence encodes a Dlx2 protein comprising an amino acid sequence at least 95% identical or similar to SEQ ID NO: 10. In one aspect, a nucleic acid coding sequence encodes a Dlx2 protein comprising an amino acid sequence at least 96% identical or similar to SEQ ID NO: 10. In one aspect, a nucleic acid coding sequence encodes a Dlx2 protein comprising an amino acid sequence at least 97% identical or similar to SEQ ID NO: 10. In one aspect, a nucleic acid coding sequence encodes a Dlx2 protein comprising an amino acid sequence at least 98% identical or similar to SEQ ID NO: 10. In one aspect, a nucleic acid coding sequence encodes a Dlx2 protein comprising an amino acid sequence at least 99% identical or similar to SEQ ID NO: 10. In one aspect, a nucleic acid coding sequence encodes a Dlx2 protein comprising an amino acid sequence at least 99.5% identical or similar to SEQ ID NO: 10. In one aspect, a nucleic acid coding sequence encodes a Dlx2 protein comprising an amino acid sequence at least 99.8% identical or similar to SEQ ID NO: 10. In one aspect, a nucleic acid coding sequence encodes a Dlx2 protein comprising an amino acid sequence at least 99.9% identical or similar to SEQ ID NO: 10. In one aspect, a nucleic acid coding sequence encodes a Dlx2 protein comprising an amino acid sequence 100% identical or similar to SEQ ID NO: 10.

Glial fibrillary acid protein (GFAP); also referred to as glial fibrillary acidic protein is a member of the type III intermediate filament family of proteins that is expressed in the central nervous system and plays a role in cell communication and the functioning of the blood-brain barrier.

In an aspect the promoter is selected from the group consisting of GFAP promoter, Sox9 promoter, S100b promoter, Aldh111 promoter, Lipocalin 2 (Lcn2) promoter, glutamine synthetase promoter, Aquaporin-4 (AQP4) promoter, oligodendrocyte transcription factor (Olig2) promoter, and synapsin promoter, NG2 promoter, ionized calcium binding adaptor molecule 1 (Iba1) promoter, cluster of differentiation 86 (CD86) promoter, platelet-derived growth factor receptor alpha (PDGFRA) promoter, platelet-derived growth factor receptor beta (PDGFRB) promoter, elongation factor 1-alpha (EF1a) promoter, CAG promoter, cytomegalovirus (CMV) promoter, ubiquitin promoter. In one aspect, the promoter is GFAP promoter. In one aspect, the promoter is Sox9 promoter. In one aspect, the promoter is Lcn2 promoter. In one aspect, the promoter is glutamine synthetase promoter. In one aspect, the promoter is AQP4 promoter. In one aspect, the promoter is Olig2 promoter. In one aspect the promoter is synapsin promoter. In one aspect, the promoter is Iba1 promoter. In one aspect, the promoter is CD86 promoter. In one aspect, the promoter is PDGFRA promoter. In one aspect, the promoter is PDGFRB promoter. In one aspect, the promoter is EF1a promoter. In one aspect, the promoter is CAG promoter. In one aspect, the promoter is CMV promoter. In one aspect, the promoter is ubiquitin promoter. In an aspect, an ubiquitin promoter is selected from the group consisting of U6, H1, 7SK, and U1. In one aspect, an ubiquitin promoter is U6. In one aspect, an ubiquitin promoter is H1. In one aspect, an ubiquitin promoter is H1. In one aspect, an ubiquitin promoter is 7SK. In one aspect, an ubiquitin promoter is U1. In one aspect, U6 comprises the nucleic acid sequence of SEQ ID NO: 17.

In an aspect, a GFAP promoter is a promoter directing astrocyte-specific expression of a protein called glial fibrillary acidic protein (GFAP) in cells. In one aspect, a GFAP promoter sequence is a human GFAP (hGFAP) promoter sequence. In one aspect, a GFAP promoter is selected from the group consisting of Gfa681, Gfa1.6, and hGFA2.2. In one aspect, a GFAP promoter is Gfa681. In one aspect, a GFAP promoter is Gfa1.6. In one aspect, a GFAP promoter is hGFA2.2. In one aspect, GFAP Gfa681 is SEQ ID NO: 3. In one aspect, GFAP Gfa1.6 is SEQ ID NO: 4. In one aspect, hGFa2.2 is SEQ ID NO: 12. In one aspect, a GFAP promoter is selected from the group consisting of SEQ ID NOs: 3, 4, and 12. In one aspect, a GFAP promoter is SEQ ID NO: 3. In one aspect, a GFAP promoter is SEQ ID NO: 4. In one aspect, a GFAP promoter is SEQ ID NO: 12.

As used herein “pGfa681” refers to a human glial fibrillary acid protein (GFAP) promoter truncated sequence of 681 bp size. “pGfa681” and “Gfa681” are used interchangeably herein.

In one aspect, a GFAP promoter sequence is selected from the group consisting of a chimpanzee GFAP promoter sequence, a bonobo GFAP promoter sequence, an orangutan GFAP promoter sequence, a gorilla GFAP promoter sequence, a macaque GFAP promoter sequence, a marmoset GFAP promoter sequence, a capuchin GFAP promoter sequence, a baboon GFAP promoter sequence, a gibbon GFAP promoter sequence, and a lemur GFAP promoter sequence. In one aspect, a GFAP promoter sequence is a chimpanzee GFAP promoter sequence. In one aspect, a GFAP promoter sequence is a bonobo GFAP promoter sequence. In one aspect, a GFAP promoter sequence is an orangutan GFAP promoter sequence. In one aspect, a GFAP promoter sequence is a gorilla GFAP promoter sequence. In one aspect, a GFAP promoter sequence is a macaque GFAP promoter sequence. In one aspect, a GFAP promoter sequence is a marmoset GFAP promoter sequence. In one aspect, a GFAP promoter sequence is a capuchin GFAP promoter sequence. In one aspect, a GFAP promoter sequence is a baboon GFAP promoter sequence. In one aspect, a GFAP promoter sequence is a gibbon GFAP promoter sequence. In one aspect, a GFAP promoter sequence is a lemur GFAP promoter sequence.

In an aspect, a GFAP promoter sequence comprises at least 100 nucleotides. In one aspect, a GFAP promoter comprises at least 500 nucleotides. In a further aspect, a GFAP promoter comprises at least 1000 nucleotides. In still another aspect, a GFAP promoter comprises at least 1500 nucleotides.

It is appreciated in the art that a fragment of a promoter sequence can function to drive transcription of an operably linked nucleic acid molecule. For example, without being limiting, if a 1000 nucleotides promoter is truncated to 500 nucleotides, and the 500 nucleotides fragment is capable of driving transcription, the 500 nucleotides fragment is referred to as a “functional fragment.”

In an aspect, a promoter comprises at least 10 nucleotides. In one aspect, a promoter comprises at least 50 nucleotides. In one aspect, a promoter comprises at least 100 nucleotides. In one aspect, an intron comprises at least 150 nucleotides. In one aspect, a promoter comprises at least 200 nucleotides. In one aspect, a promoter comprises at least 250 nucleotides. In one aspect, a promoter comprises at least 300 nucleotides. In one aspect, a promoter comprises at least 350 nucleotides. In one aspect, a promoter comprises at least 400 nucleotides. In one aspect, a promoter comprises at least 450 nucleotides. In one aspect, a promoter comprises at least 500 nucleotides. In one aspect, a promoter comprises between 50 nucleotides and 7500 nucleotides. In one aspect, a promoter comprises between 50 nucleotides and 5000 nucleotides. In one aspect, a promoter comprises between 50 nucleotides and 2500 nucleotides. In one aspect, a promoter comprises between 50 nucleotides and 1000 nucleotides. In one aspect, a promoter comprises between 50 nucleotides and 500 nucleotides. In one aspect, a promoter comprises between 10 nucleotides and 7500 nucleotides. In one aspect, a promoter comprises between 10 nucleotides and 5000 nucleotides. In one aspect, a promoter comprises between 10 nucleotides and 2500 nucleotides. In one aspect, a promoter comprises between 10 nucleotides and 1000 nucleotides. In one aspect, a promoter comprises between 10 nucleotides and 500 nucleotides

In an aspect, a GFAP promoter nucleic acid sequence comprises a sequence at least 70% identical to a sequence selected from the group consisting of SEQ ID NOs: 3, 4, 12, and functional fragment thereof. In one aspect, a GFAP promoter nucleic acid sequence comprises a sequence at least 75% identical to a sequence selected from the group consisting of SEQ ID NOs: 3, 4, 12, and functional fragment thereof. In one aspect, a GFAP promoter nucleic acid sequence comprises a sequence at least 80% identical to a sequence selected from the group consisting of SEQ ID NOs: 3, 4, 12, and functional fragment thereof. In one aspect, a GFAP promoter nucleic acid sequence comprises a sequence at least 85% identical to a sequence selected from the group consisting of SEQ ID NOs: 3, 4, 12, and functional fragment thereof. In one aspect, a GFAP promoter nucleic acid sequence comprises a sequence at least 90% identical to a sequence selected from the group consisting of SEQ ID NOs: 3, 4, 12, and functional fragment thereof. In one aspect, a GFAP promoter nucleic acid sequence comprises a sequence at least 91% identical to a sequence selected from the group consisting of SEQ ID NOs: 3, 4, 12, and functional fragment thereof. In one aspect, a GFAP promoter nucleic acid sequence comprises a sequence at least 92% identical to a sequence selected from the group consisting of SEQ ID NOs: 3, 4, 12, and functional fragment thereof. In one aspect, a GFAP promoter nucleic acid sequence comprises a sequence at least 93% identical to a sequence selected from the group consisting of SEQ ID NOs: 3, 4, 12, and functional fragment thereof. In one aspect, a GFAP promoter nucleic acid sequence comprises a sequence at least 94% identical to a sequence selected from the group consisting of SEQ ID NOs: 3, 4, 12, and functional fragment thereof. In one aspect, a GFAP promoter nucleic acid sequence comprises a sequence at least 95% identical to a sequence selected from the group consisting of SEQ ID NOs: 3, 4, 12, and functional fragment thereof. In one aspect, a GFAP promoter nucleic acid sequence comprises a sequence at least 96% identical to a sequence selected from the group consisting of SEQ ID NOs: 3, 4, 12, and functional fragment thereof. In one aspect, a GFAP promoter nucleic acid sequence comprises a sequence at least 97% identical to a sequence selected from the group consisting of SEQ ID NOs: 3, 4, 12, and functional fragment thereof. In one aspect, a GFAP promoter nucleic acid sequence comprises a sequence at least 98% identical to a sequence selected from the group consisting of SEQ ID NOs: 3, 4, 12, and functional fragment thereof. In one aspect, a GFAP promoter nucleic acid sequence comprises a sequence at least 99% identical to a sequence selected from the group consisting of SEQ ID NOs: 3, 4, 12, and functional fragment thereof. In one aspect, a GFAP promoter nucleic acid sequence comprises a sequence at least 99.5% identical to a sequence selected from the group consisting of SEQ ID NOs: 3, 4, 12, and functional fragment thereof. In one aspect, a GFAP promoter nucleic acid sequence comprises a sequence at least 99.8% identical to a sequence selected from the group consisting of SEQ ID NOs: 3, 4, 12, and functional fragment thereof. In one aspect, a GFAP promoter nucleic acid sequence comprises a sequence at least 99.9% identical to a sequence selected from the group consisting of SEQ ID NOs: 3, 4, 12, and functional fragment thereof. In one aspect, a GFAP promoter nucleic acid sequence comprises a sequence 100% identical to a sequence selected from the group consisting of SEQ ID NOs: 3, 4, 12, and functional fragment thereof.

In an aspect, a nucleic acid sequence as provided herein is codon optimized.

In an aspect, a nucleic acid sequence as provided herein is CpG site depleted.

As used herein, the term “brain” refers to an organ that functions as the center of the nervous system. In an aspect, a brain comprises a cerebrum, a cerebral cortex, a cerebellum, and/or a brain stem.

As used herein, the term “cerebral cortex” refers to the outer layer of neural tissue of the cerebrum.

As used herein, the term “striatum” or “corpus striatum” refers to a cluster of neurons in the subcortical basal ganglia of the forebrain and comprises the ventral striatum and dorsal striatum.

As used herein, the term “substantia nigra” refers to a cluster of neurons in the subcortical basal ganglia of the midbrain and comprises the pars compacta and the pars reticulata.

As used herein, the term “forebrain” refers to the forward-most portion of the brain.

As used herein, the term “putamen” refers to a round structure at the base of the forebrain and is a component of the dorsal striatum.

As used herein, the term “caudate nucleus” refers to a structure at the base of the forebrain and is a component of the dorsal striatum.

As used herein, the term “subcortical basal ganglia” refers to a cluster of neurons in the deep cerebral hemispheres of the brain.

As used herein, the term “spinal cord” refers to a structure that functions in the transmission of nerve signals from the motor cortex to the body.

As used herein, the term “motor cortex” refers to a region in the frontal lobe of the cerebral cortex that is involved in the planning, control, and execution of voluntary movements.

In an aspect, a method provided herein converts reactive astrocytes to functional neurons in the brain. In one aspect, a method provided herein converts reactive astrocytes to functional neurons in a cerebral cortex of the brain. In one aspect, a method provided herein coverts reactive astrocytes to functional neurons in a striatum of the brain. In one aspect, a method provided herein converts reactive astrocytes to functional neurons in a dorsal striatum of the brain. In one aspect, a method provided herein converts reactive astrocytes to functional neurons in a spinal cord of the brain. In one aspect, a method provided herein converts reactive astrocytes to functional neurons in a putamen of the brain. In one aspect, a method provided herein converts reactive astrocytes to functional neurons in a caudate nucleus of the brain. In one aspect, a method provided herein converts reactive astrocytes to functional neurons in a substantia nigra of the brain.

Elongation factor-1 alpha (EF-1 alpha; also referred to as eEF1a1) is an isoform of the alpha subunit of the elongation factor 1 complex. The complex is involved in the enzymatic delivery of aminoacyl tRNAs to the ribosome. The EF-1 alpha isoform is expressed in the brain, placenta, lung, liver, kidney, and pancreas.

In an aspect, an enhancer sequence from the EF-1 alpha promoter is a human enhancer sequence from the EF-1 alpha promoter. In one aspect, an enhancer sequence from the EF-1 alpha promoter is selected form the group consisting of a chimpanzee enhancer sequence from the EF-1 alpha promoter, a bonobo enhancer sequence from the EF-1 alpha promoter, an orangutan enhancer sequence from the EF-1 alpha promoter, a gorilla enhancer sequence from the EF-1 alpha promoter, a macaque enhancer sequence from the EF-1 alpha promoter, a marmoset enhancer sequence from the EF-1 alpha promoter, a capuchin enhancer sequence from the EF-1 alpha promoter, a baboon enhancer sequence from the EF-1 alpha promoter, a gibbon enhancer sequence from the EF-1 alpha promoter, and a lemur enhancer sequence from the EF-1 alpha promoter. In one aspect, an enhancer sequence from the EF-1 alpha promoter is a chimpanzee an enhancer sequence from the EF-1 alpha promoter. In one aspect, an enhancer sequence from the EF-1 alpha promoter is a bonobo enhancer sequence from the EF-1 alpha promoter. In one aspect, an enhancer sequence from the EF-1 alpha promoter is an orangutan enhancer sequence from the EF-1 alpha promoter. In one aspect, an enhancer sequence from the EF-1 alpha promoter is a gorilla enhancer sequence from the EF-1 alpha promoter. In one aspect, an enhancer sequence from the EF-1 alpha promoter is a macaque enhancer sequence from the EF-1 alpha promoter. In one aspect, enhancer sequence from the EF-1 alpha promoter is a marmoset enhancer sequence from the EF-1 alpha promoter. In one aspect, enhancer sequence from the EF-1 alpha promoter is a capuchin enhancer sequence from the EF-1 alpha promoter. In one aspect, enhancer sequence from the EF-1 alpha promoter is a baboon enhancer sequence from the EF-1 alpha promoter. In one aspect, enhancer sequence from the EF-1 alpha promoter is a gibbon enhancer sequence from the EF-1 alpha promoter. In one aspect, enhancer sequence from the EF-1 alpha promoter is a lemur enhancer sequence from the EF-1 alpha promoter.

In an aspect, an enhancer from the EF-1 alpha promoter nucleic acid sequence comprises a sequence at least 70% identical to SEQ ID NO: 2, or the complement thereof. In one aspect, an enhancer from the EF-1 alpha promoter nucleic acid sequence comprises a sequence at least 75% identical to SEQ ID NO: 2, or the complement thereof. In one aspect, an enhancer from the EF-1 alpha promoter nucleic acid sequence comprises a sequence at least 80% identical to SEQ ID NO: 2, or the complement thereof. In one aspect, an enhancer from the EF-1 alpha promoter nucleic acid sequence comprises a sequence at least 85% identical to SEQ ID NO: 2, or the complement thereof. In one aspect, an enhancer from the EF-1 alpha promoter nucleic acid sequence comprises a sequence at least 90% identical to SEQ ID NO: 2, or the complement thereof. In one aspect, an enhancer from the EF-1 alpha promoter nucleic acid sequence comprises a sequence at least 91% identical to SEQ ID NO: 2, or the complement thereof. In one aspect, an enhancer from the EF-1 alpha promoter nucleic acid sequence comprises a sequence at least 92% identical to SEQ ID NO: 2, or the complement thereof. In one aspect, an enhancer from the EF-1 alpha promoter nucleic acid sequence comprises a sequence at least 93% identical to SEQ ID NO: 2, or the complement thereof. In one aspect, an enhancer from the EF-1 alpha promoter nucleic acid sequence comprises a sequence at least 94% identical to SEQ ID NO: 2, or the complement thereof. In one aspect, an enhancer from the EF-1 alpha promoter nucleic acid sequence comprises a sequence at least 95% identical to SEQ ID NO: 2, or the complement thereof. In one aspect, an enhancer from the EF-1 alpha promoter nucleic acid sequence comprises a sequence at least 96% identical to SEQ ID NO: 2, or the complement thereof. In one aspect, an enhancer from the EF-1 alpha promoter nucleic acid sequence comprises a sequence at least 97% identical to SEQ ID NO: 2, or the complement thereof. In one aspect, an enhancer from the EF-1 alpha promoter nucleic acid sequence comprises a sequence at least 98% identical to SEQ ID NO: 2, or the complement thereof. In one aspect, an enhancer from the EF-1 alpha promoter nucleic acid sequence comprises a sequence at least 99% identical to SEQ ID NO: 2, or the complement thereof. In one aspect, an enhancer from the EF-1 alpha promoter nucleic acid sequence comprises a sequence at least 99.5% identical to SEQ ID NO: 2, or the complement thereof. In one aspect, an enhancer from the EF-1 alpha promoter nucleic acid sequence comprises a sequence at least 99.8% identical to SEQ ID NO: 2, or the complement thereof. In one aspect, an enhancer from the EF-1 alpha promoter nucleic acid sequence comprises a sequence at least 99.9% identical to SEQ ID NO: 2, or the complement thereof. In one aspect, an enhancer from the EF-1 alpha promoter nucleic acid sequence comprises a sequence 100% identical to SEQ ID NO: 2, or the complement thereof.

Cytomegalovirus (CMV) is a genus of viruses in the order Herpesvirale.

In an aspect, an enhancer sequence from the CMV is a human enhancer sequence from the CMV. In one aspect, an enhancer sequence from the CMV is selected form the group consisting of a chimpanzee enhancer sequence from the CMV, a bonobo enhancer sequence from the CMV, an orangutan enhancer sequence from the CMV, a gorilla enhancer sequence from the CMV, a macaque enhancer sequence from the CMV, a marmoset enhancer sequence from the CMV, a capuchin enhancer sequence from the CMV, a baboon enhancer sequence from the CMV, a gibbon enhancer sequence from the CMV, and a lemur enhancer sequence from the CMV. In one aspect, an enhancer sequence from the CMV is a chimpanzee an enhancer sequence from the CMV. In one aspect, an enhancer sequence from the CMV is a bonobo enhancer sequence from the CMV. In one aspect, an enhancer sequence from the CMV is an orangutan enhancer sequence from the CMV. In one aspect, an enhancer sequence from the CMV is a gorilla enhancer sequence from the CMV. In one aspect, an enhancer sequence from the CMV is a macaque enhancer sequence from the CMV. In one aspect, enhancer sequence from the CMV is a marmoset enhancer sequence from the CMV. In one aspect, enhancer sequence from the CMV is a capuchin enhancer sequence from the CMV. In one aspect, enhancer sequence from the CMV is a baboon enhancer sequence from the CMV. In one aspect, enhancer sequence from the CMV is a gibbon enhancer sequence from the CMV. In one aspect, enhancer sequence from the CMV is a lemur enhancer sequence from the CMV.

In an aspect, an enhancer from the CMV nucleic acid sequence comprises a sequence at least 70% identical to SEQ ID NO: 11, or the complement thereof. In one aspect, an enhancer from the CMV nucleic acid sequence comprises a sequence at least 75% identical to SEQ ID NO: 11, or the complement thereof. In one aspect, an enhancer from the CMV nucleic acid sequence comprises a sequence at least 80% identical to SEQ ID NO: 11, or the complement thereof. In one aspect, an enhancer from the CMV nucleic acid sequence comprises a sequence at least 85% identical to SEQ ID NO: 11, or the complement thereof. In one aspect, an enhancer from the CMV nucleic acid sequence comprises a sequence at least 90% identical to SEQ ID NO: 11, or the complement thereof. In one aspect, an enhancer from the CMV nucleic acid sequence comprises a sequence at least 91% identical to SEQ ID NO: 11, or the complement thereof. In one aspect, an enhancer from the CMV nucleic acid sequence comprises a sequence at least 92% identical to SEQ ID NO: 11, or the complement thereof. In one aspect, an enhancer from the CMV nucleic acid sequence comprises a sequence at least 93% identical to SEQ ID NO: 11, or the complement thereof. In one aspect, an enhancer from the CMV nucleic acid sequence comprises a sequence at least 94% identical to SEQ ID NO: 11, or the complement thereof. In one aspect, an enhancer from the CMV nucleic acid sequence comprises a sequence at least 95% identical to SEQ ID NO: 11, or the complement thereof. In one aspect, an enhancer from the CMV nucleic acid sequence comprises a sequence at least 96% identical to SEQ ID NO: 11, or the complement thereof. In one aspect, an enhancer from the CMV nucleic acid sequence comprises a sequence at least 97% identical to SEQ ID NO: 11, or the complement thereof. In one aspect, an enhancer from the CMV nucleic acid sequence comprises a sequence at least 98% identical to SEQ ID NO: 11, or the complement thereof. In one aspect, an enhancer from the CMV nucleic acid sequence comprises a sequence at least 99% identical to SEQ ID NO: 11, or the complement thereof. In one aspect, an enhancer from the CMV nucleic acid sequence comprises a sequence at least 99.5% identical to SEQ ID NO: 11, or the complement thereof. In one aspect, an enhancer from the CMV nucleic acid sequence comprises a sequence at least 99.8% identical to SEQ ID NO: 11, or the complement thereof. In one aspect, an enhancer from the CMV nucleic acid sequence comprises a sequence at least 99.9% identical to SEQ ID NO: 11, or the complement thereof. In one aspect, an enhancer from the CMV nucleic acid sequence comprises a sequence 100% identical to SEQ ID NO: 11, or the complement thereof.

In an aspect, an enhancer is selected from the group consisting of an enhancer from EF1-αpromoter and CMV enhancer. In one aspect, an enhancer is from EF1-αpromoter. In one aspect, an enhancer is an CMV enhancer.

Introns can be grouped into at least five classes, including: spliceosomal introns; transfer RNA introns; group I introns; group II introns; and group III introns. An intron can be synthetically produced, varied, or derived from a known or naturally occurring intron sequence or other intron sequence. An intron can also include a chimeric intron comprising a combination of two or more heterologous sequences. An intron of the present application can thus include variants of intron sequences that are similar in composition, but not identical to, other intron sequence(s) known or provided herein. In an aspect, an intron comprises at least 10 nucleotides. In one aspect, an intron comprises at least 50 nucleotides. In one aspect, an intron comprises at least 100 nucleotides. In one aspect, an intron comprises at least 150 nucleotides. In one aspect, an intron comprises at least 200 nucleotides. In one aspect, an intron comprises at least 250 nucleotides. In one aspect, an intron comprises at least 300 nucleotides. In one aspect, an intron comprises at least 350 nucleotides. In one aspect, an intron comprises at least 400 nucleotides. In one aspect, an intron comprises at least 450 nucleotides. In one aspect, an intron comprises at least 500 nucleotides. In one aspect, an intron comprises between 50 nucleotides and 7500 nucleotides. In one aspect, an intron comprises between 50 nucleotides and 5000 nucleotides. In one aspect, an intron comprises between 50 nucleotides and 2500 nucleotides. In one aspect, an intron comprises between 50 nucleotides and 1000 nucleotides. In one aspect, an intron comprises between 50 nucleotides and 500 nucleotides. In one aspect, an intron comprises between 10 nucleotides and 7500 nucleotides. In one aspect, an intron comprises between 10 nucleotides and 5000 nucleotides. In one aspect, an intron comprises between 10 nucleotides and 2500 nucleotides. In one aspect, an intron comprises between 10 nucleotides and 1000 nucleotides. In one aspect, an intron comprises between 10 nucleotides and 500 nucleotides.

As used herein “CI” refers to a chimeric intron composed of the 5′-donor site from the first intron of the human β-globin gene and the branch and 3′-acceptor site from the intron of an immunoglobulin gene heavy chain variable region.

As used herein “CRGI” refers to a chimeric intron of rabbit beta-globing and chicken beta actin similar in CAG promoter.

In an aspect, a chimeric intron nucleic acid sequence is selected from the group consisting of SEQ ID NOs: 5 and 19. In one aspect, a chimeric intron nucleic acid sequence is SEQ ID NO: 5. In one aspect, a chimeric intron nucleic acid sequence is SEQ ID NO: 19.

In an aspect, a chimeric intron nucleic acid sequence comprises a sequence at least 70% identical to SEQ ID NO: 5, or the complement thereof. In one aspect, a chimeric intron nucleic acid sequence comprises a sequence at least 75% identical to SEQ ID NO: 5, or the complement thereof. In one aspect, a chimeric intron nucleic acid sequence comprises a sequence at least 80% identical to SEQ ID NO: 5, or the complement thereof. In one aspect, a chimeric intron nucleic acid sequence comprises a sequence at least 85% identical to SEQ ID NO: 5, or the complement thereof. In one aspect, a chimeric intron nucleic acid sequence comprises a sequence at least 90% identical to SEQ ID NO: 5, or the complement thereof. In one aspect, a chimeric intron nucleic acid sequence comprises a sequence at least 91% identical to SEQ ID NO: 5, or the complement thereof. In one aspect, a chimeric intron nucleic acid sequence comprises a sequence at least 92% identical to SEQ ID NO: 5, or the complement thereof. In one aspect, a chimeric intron nucleic acid sequence comprises a sequence at least 93% identical to SEQ ID NO: 5, or the complement thereof. In one aspect, a chimeric intron nucleic acid sequence comprises a sequence at least 94% identical to SEQ ID NO: 5, or the complement thereof. In one aspect, a chimeric intron nucleic acid sequence comprises a sequence at least 95% identical to SEQ ID NO: 5, or the complement thereof. In one aspect, a chimeric intron nucleic acid sequence comprises a sequence at least 96% identical to SEQ ID NO: 5, or the complement thereof. In one aspect, a chimeric intron nucleic acid sequence comprises a sequence at least 97% identical to SEQ ID NO: 5, or the complement thereof. In one aspect, a chimeric intron nucleic acid sequence comprises a sequence at least 98% identical to SEQ ID NO: 5, or the complement thereof. In one aspect, a chimeric intron nucleic acid sequence comprises a sequence at least 99% identical to SEQ ID NO: 5, or the complement thereof. In one aspect, a chimeric intron nucleic acid sequence comprises a sequence at least 99.5% identical to SEQ ID NO: 5, or the complement thereof. In one aspect, a chimeric intron nucleic acid sequence comprises a sequence at least 99.8% identical to SEQ ID NO: 5, or the complement thereof. In one aspect, a chimeric intron nucleic acid sequence comprises a sequence at least 99.9% identical to SEQ ID NO: 5, or the complement thereof. In one aspect, a chimeric intron nucleic acid sequence comprises a sequence 100% identical to SEQ ID NO: 5, or the complement thereof.

In an aspect, a chimeric intron is a chimeric intron of rabbit beta-globing and chicken beta actin similar in CAG promoter (CRGI). In an aspect, the CRGI sequence comprises SEQ ID NO: 19. In an aspect, a chimeric intron nucleic acid sequence comprises a sequence at least 70% identical to SEQ ID NO: 19, or the complement thereof. In one aspect, a chimeric intron nucleic acid sequence comprises a sequence at least 75% identical to SEQ ID NO: 19, or the complement thereof. In one aspect, a chimeric intron nucleic acid sequence comprises a sequence at least 80% identical to SEQ ID NO: 19, or the complement thereof. In one aspect, a chimeric intron nucleic acid sequence comprises a sequence at least 85% identical to SEQ ID NO: 19, or the complement thereof. In one aspect, a chimeric intron nucleic acid sequence comprises a sequence at least 90% identical to SEQ ID NO: 19, or the complement thereof. In one aspect, a chimeric intron nucleic acid sequence comprises a sequence at least 91% identical to SEQ ID NO: 19, or the complement thereof. In one aspect, a chimeric intron nucleic acid sequence comprises a sequence at least 92% identical to SEQ ID NO: 19, or the complement thereof. In one aspect, a chimeric intron nucleic acid sequence comprises a sequence at least 93% identical to SEQ ID NO: 19, or the complement thereof. In one aspect, a chimeric intron nucleic acid sequence comprises a sequence at least 94% identical to SEQ ID NO: 19, or the complement thereof. In one aspect, a chimeric intron nucleic acid sequence comprises a sequence at least 95% identical to SEQ ID NO: 19, or the complement thereof. In one aspect, a chimeric intron nucleic acid sequence comprises a sequence at least 96% identical to SEQ ID NO: 19, or the complement thereof. In one aspect, a chimeric intron nucleic acid sequence comprises a sequence at least 97% identical to SEQ ID NO: 19, or the complement thereof. In one aspect, a chimeric intron nucleic acid sequence comprises a sequence at least 98% identical to SEQ ID NO: 19, or the complement thereof. In one aspect, a chimeric intron nucleic acid sequence comprises a sequence at least 99% identical to SEQ ID NO: 19, or the complement thereof. In one aspect, a chimeric intron nucleic acid sequence comprises a sequence at least 99.5% identical to SEQ ID NO: 19, or the complement thereof. In one aspect, a chimeric intron nucleic acid sequence comprises a sequence at least 99.8% identical to SEQ ID NO: 19, or the complement thereof. In one aspect, a chimeric intron nucleic acid sequence comprises a sequence at least 99.9% identical to SEQ ID NO: 19, or the complement thereof. In one aspect, a chimeric intron nucleic acid sequence comprises a sequence 100% identical to SEQ ID NO: 19, or the complement thereof.

The woodchuck hepatitis virus posttranscriptional regulatory element (WPRE) is a DNA sequence that creates a tertiary structure enhancing expression of genes that are delivered in viral vectors.

As used herein “WPRE” refers to a Woodchuck Hepatitis Virus (WHV) Posttranscriptional Regulatory Element.

As used herein “oPRE” refers to an optimized version of WPRE.

In an aspect, a WPRE nucleic acid sequence is selected from the group consisting of SEQ ID NOs: 7 and 18. In one aspect, a WPRE nucleic acid sequence is SEQ ID NO: 7. In one aspect, a WPRE nucleic acid sequence is SEQ ID NO: 18.

In an aspect, a WPRE nucleic acid sequence comprises a sequence at least 70% identical to SEQ ID NO: 7, or the complement thereof. In one aspect, a WPRE nucleic acid sequence comprises a sequence at least 75% identical to SEQ ID NO: 7, or the complement thereof. In one aspect, a WPRE nucleic acid sequence comprises a sequence at least 80% identical to SEQ ID NO: 7, or the complement thereof. In one aspect, a WPRE nucleic acid sequence comprises a sequence at least 85% identical to SEQ ID NO: 7, or the complement thereof. In one aspect, a WPRE nucleic acid sequence comprises a sequence at least 90% identical to SEQ ID NO: 7, or the complement thereof. In one aspect, a WPRE nucleic acid sequence comprises a sequence at least 91% identical to SEQ ID NO: 7, or the complement thereof. In one aspect, a WPRE nucleic acid sequence comprises a sequence at least 92% identical to SEQ ID NO: 7, or the complement thereof. In one aspect, a WPRE nucleic acid sequence comprises a sequence at least 93% identical to SEQ ID NO: 7, or the complement thereof. In one aspect, a WPRE nucleic acid sequence comprises a sequence at least 94% identical to SEQ ID NO: 7, or the complement thereof. In one aspect, a WPRE nucleic acid sequence comprises a sequence at least 95% identical to SEQ ID NO: 7, or the complement thereof. In one aspect, a WPRE nucleic acid sequence comprises a sequence at least 96% identical to SEQ ID NO: 7, or the complement thereof. In one aspect, a WPRE nucleic acid sequence comprises a sequence at least 97% identical to SEQ ID NO: 7, or the complement thereof. In one aspect, a WPRE nucleic acid sequence comprises a sequence at least 98% identical to SEQ ID NO: 7, or the complement thereof. In one aspect, a WPRE nucleic acid sequence comprises a sequence at least 99% identical to SEQ ID NO: 7, or the complement thereof. In one aspect, a WPRE nucleic acid sequence comprises a sequence at least 99.5% identical to SEQ ID NO: 7, or the complement thereof. In one aspect, a WPRE nucleic acid sequence comprises a sequence at least 99.8% identical to SEQ ID NO: 7, or the complement thereof. In one aspect, a WPRE nucleic acid sequence comprises a sequence at least 99.9% identical to SEQ ID NO: 7, or the complement thereof. In one aspect, a WPRE nucleic acid sequence comprises a sequence 100% identical to SEQ ID NO: 7, or the complement thereof.

In an aspect, a WPRE nucleic acid sequence comprises a sequence at least 70% identical to SEQ ID NO: 18, or the complement thereof. In one aspect, a WPRE nucleic acid sequence comprises a sequence at least 75% identical to SEQ ID NO: 18, or the complement thereof. In one aspect, a WPRE nucleic acid sequence comprises a sequence at least 80% identical to SEQ ID NO: 18, or the complement thereof. In one aspect, a WPRE nucleic acid sequence comprises a sequence at least 85% identical to SEQ ID NO: 18, or the complement thereof. In one aspect, a WPRE nucleic acid sequence comprises a sequence at least 90% identical to SEQ ID NO: 18, or the complement thereof. In one aspect, a WPRE nucleic acid sequence comprises a sequence at least 91% identical to SEQ ID NO: 18, or the complement thereof. In one aspect, a WPRE nucleic acid sequence comprises a sequence at least 92% identical to SEQ ID NO: 18, or the complement thereof. In one aspect, a WPRE nucleic acid sequence comprises a sequence at least 93% identical to SEQ ID NO: 18, or the complement thereof. In one aspect, a WPRE nucleic acid sequence comprises a sequence at least 94% identical to SEQ ID NO: 18, or the complement thereof. In one aspect, a WPRE nucleic acid sequence comprises a sequence at least 95% identical to SEQ ID NO: 18, or the complement thereof. In one aspect, a WPRE nucleic acid sequence comprises a sequence at least 96% identical to SEQ ID NO: 18, or the complement thereof. In one aspect, a WPRE nucleic acid sequence comprises a sequence at least 97% identical to SEQ ID NO: 18, or the complement thereof. In one aspect, a WPRE nucleic acid sequence comprises a sequence at least 98% identical to SEQ ID NO: 18, or the complement thereof. In one aspect, a WPRE nucleic acid sequence comprises a sequence at least 99% identical to SEQ ID NO: 18, or the complement thereof. In one aspect, a WPRE nucleic acid sequence comprises a sequence at least 99.5% identical to SEQ ID NO: 18, or the complement thereof. In one aspect, a WPRE nucleic acid sequence comprises a sequence at least 99.8% identical to SEQ ID NO: 18, or the complement thereof. In one aspect, a WPRE nucleic acid sequence comprises a sequence at least 99.9% identical to SEQ ID NO: 18, or the complement thereof. In one aspect, a WPRE nucleic acid sequence comprises a sequence 100% identical to SEQ ID NO: 18, or the complement thereof.

SV40 polyadenylation signal sequence (also refer as SV40 PolyA; Simian virus 40 PolyA; and PolyA) is a DNA sequence the can terminate transcription and add a PolyA tail to the 3′ end of a messenger RNA (mRNA).

hGH polyadenylation signal sequence (also refer as hGH PolyA) is a DNA sequence the can terminate transcription and add a PolyA tail to the 3′ end of a messenger RNA (mRNA).

As used herein “SV40pA” refers to a poly A signal of SV40 virus.

As used herein “bGHpA” refers to a poly A signal of bovine growth hormone.

As used herein, a “PolyA tail” refers to a stretch of RNA that only contains the nucleobase adenine. In an aspect, an RNA molecule transcribed from an AAV vector construct provided herein comprises a PolyA tail. In one aspect, a PolyA tail comprises at least two adenines. In one aspect, a PolyA tail comprises at least ten adenines. In one aspect, a PolyA tail comprises at least 50 adenines. In one aspect, a PolyA tail comprises at least 100 adenines. In one aspect, a PolyA tail comprises at least 150 adenines. In one aspect, a PolyA tail comprises at least 200 adenines. In one aspect, a PolyA tail comprises at least 250 adenines. In one aspect, a PolyA tail comprises between 50 adenines and 300 adenines.

In an aspect, a SV40 polyadenylation signal nucleic acid sequence comprises a sequence at least 70% identical to SEQ ID NO: 8, or the complement thereof. In one aspect, a SV40 polyadenylation signal nucleic acid sequence comprises a sequence at least 75% identical to SEQ ID NO: 8, or the complement thereof. In one aspect, a SV40 polyadenylation signal nucleic acid sequence comprises a sequence at least 80% identical to SEQ ID NO: 8, or the complement thereof. In one aspect, a SV40 polyadenylation signal nucleic acid sequence comprises a sequence at least 85% identical to SEQ ID NO: 8, or the complement thereof. In one aspect, a SV40 polyadenylation signal nucleic acid sequence comprises a sequence at least 90% identical to SEQ ID NO: 8, or the complement thereof. In one aspect, a SV40 polyadenylation signal nucleic acid sequence comprises a sequence at least 91% identical to SEQ ID NO: 8, or the complement thereof. In one aspect, a SV40 polyadenylation signal nucleic acid sequence comprises a sequence at least 92% identical to SEQ ID NO: 8, or the complement thereof. In one aspect, a SV40 polyadenylation signal nucleic acid sequence comprises a sequence at least 93% identical to SEQ ID NO: 8, or the complement thereof. In one aspect, a SV40 polyadenylation signal nucleic acid sequence comprises a sequence at least 94% identical to SEQ ID NO: 8, or the complement thereof. In one aspect, a SV40 polyadenylation signal nucleic acid sequence comprises a sequence at least 95% identical to SEQ ID NO: 8, or the complement thereof. In one aspect, a SV40 polyadenylation signal nucleic acid sequence comprises a sequence at least 96% identical to SEQ ID NO: 8, or the complement thereof. In one aspect, a SV40 polyadenylation signal nucleic acid sequence comprises a sequence at least 97% identical to SEQ ID NO: 8, or the complement thereof. In one aspect, a SV40 polyadenylation signal nucleic acid sequence comprises a sequence at least 98% identical to SEQ ID NO: 8, or the complement thereof. In one aspect, a SV40 polyadenylation signal nucleic acid sequence comprises a sequence at least 99% identical to SEQ ID NO: 8, or the complement thereof. In one aspect, a SV40 polyadenylation signal nucleic acid sequence comprises a sequence at least 99.5% identical to SEQ ID NO: 8, or the complement thereof. In one aspect, a SV40 polyadenylation signal nucleic acid sequence comprises a sequence at least 99.8% identical to SEQ ID NO: 8, or the complement thereof. In one aspect, a SV40 polyadenylation signal nucleic acid sequence comprises a sequence at least 99.9% identical to SEQ ID NO: 8, or the complement thereof. In one aspect, a SV40 polyadenylation signal nucleic acid sequence comprises a sequence 100% identical to SEQ ID NO: 8, or the complement thereof.

In an aspect, a hGH polyadenylation signal nucleic acid sequence comprises a sequence at least 70% identical to SEQ ID NO: 13, or the complement thereof. In one aspect, a hGH polyadenylation signal nucleic acid sequence comprises a sequence at least 75% identical to SEQ ID NO: 13, or the complement thereof. In one aspect, a hGH polyadenylation signal nucleic acid sequence comprises a sequence at least 130% identical to SEQ ID NO: 13, or the complement thereof. In one aspect, a hGH polyadenylation signal nucleic acid sequence comprises a sequence at least 135% identical to SEQ ID NO: 13, or the complement thereof. In one aspect, a hGH polyadenylation signal nucleic acid sequence comprises a sequence at least 90% identical to SEQ ID NO: 13, or the complement thereof. In one aspect, a hGH polyadenylation signal nucleic acid sequence comprises a sequence at least 91% identical to SEQ ID NO: 13, or the complement thereof. In one aspect, a hGH polyadenylation signal nucleic acid sequence comprises a sequence at least 92% identical to SEQ ID NO: 13, or the complement thereof. In one aspect, a hGH polyadenylation signal nucleic acid sequence comprises a sequence at least 93% identical to SEQ ID NO: 13, or the complement thereof. In one aspect, a hGH polyadenylation signal nucleic acid sequence comprises a sequence at least 94% identical to SEQ ID NO: 13, or the complement thereof. In one aspect, a hGH polyadenylation signal nucleic acid sequence comprises a sequence at least 95% identical to SEQ ID NO: 13, or the complement thereof. In one aspect, a hGH polyadenylation signal nucleic acid sequence comprises a sequence at least 96% identical to SEQ ID NO: 13, or the complement thereof. In one aspect, a hGH polyadenylation signal nucleic acid sequence comprises a sequence at least 97% identical to SEQ ID NO: 13, or the complement thereof. In one aspect, a hGH polyadenylation signal nucleic acid sequence comprises a sequence at least 98% identical to SEQ ID NO: 13, or the complement thereof. In one aspect, a hGH polyadenylation signal nucleic acid sequence comprises a sequence at least 99% identical to SEQ ID NO: 13, or the complement thereof. In one aspect, a hGH polyadenylation signal nucleic acid sequence comprises a sequence at least 99.5% identical to SEQ ID NO: 13, or the complement thereof. In one aspect, a hGH polyadenylation signal nucleic acid sequence comprises a sequence at least 99.13% identical to SEQ ID NO: 13, or the complement thereof. In one aspect, a hGH polyadenylation signal nucleic acid sequence comprises a sequence at least 99.9% identical to SEQ ID NO: 13, or the complement thereof. In one aspect, a hGH polyadenylation signal nucleic acid sequence comprises a sequence 100% identical to SEQ ID NO: 13, or the complement thereof.

In an aspect, a bGH polyadenylation signal nucleic acid sequence comprises a sequence at least 70% identical to SEQ ID NO: 20, or the complement thereof. In one aspect, a bGH polyadenylation signal nucleic acid sequence comprises a sequence at least 75% identical to SEQ ID NO: 20, or the complement thereof. In one aspect, a bGH polyadenylation signal nucleic acid sequence comprises a sequence at least 80% identical to SEQ ID NO: 20, or the complement thereof. In one aspect, a bGH polyadenylation signal nucleic acid sequence comprises a sequence at least 85% identical to SEQ ID NO: 20, or the complement thereof. In one aspect, a bGH polyadenylation signal nucleic acid sequence comprises a sequence at least 90% identical to SEQ ID NO: 20, or the complement thereof. In one aspect, a bGH polyadenylation signal nucleic acid sequence comprises a sequence at least 91% identical to SEQ ID NO: 20, or the complement thereof. In one aspect, a bGH polyadenylation signal nucleic acid sequence comprises a sequence at least 92% identical to SEQ ID NO: 20, or the complement thereof. In one aspect, a bGH polyadenylation signal nucleic acid sequence comprises a sequence at least 93% identical to SEQ ID NO: 20, or the complement thereof. In one aspect, a bGH polyadenylation signal nucleic acid sequence comprises a sequence at least 94% identical to SEQ ID NO: 20, or the complement thereof. In one aspect, a bGH polyadenylation signal nucleic acid sequence comprises a sequence at least 95% identical to SEQ ID NO: 20, or the complement thereof. In one aspect, a bGH polyadenylation signal nucleic acid sequence comprises a sequence at least 96% identical to SEQ ID NO: 20, or the complement thereof. In one aspect, a bGH polyadenylation signal nucleic acid sequence comprises a sequence at least 97% identical to SEQ ID NO: 20, or the complement thereof. In one aspect, a bGH polyadenylation signal nucleic acid sequence comprises a sequence at least 98% identical to SEQ ID NO: 20, or the complement thereof. In one aspect, a bGH polyadenylation signal nucleic acid sequence comprises a sequence at least 99% identical to SEQ ID NO: 20, or the complement thereof. In one aspect, a bGH polyadenylation signal nucleic acid sequence comprises a sequence at least 99.5% identical to SEQ ID NO: 20, or the complement thereof. In one aspect, a bGH polyadenylation signal nucleic acid sequence comprises a sequence at least 99.13% identical to SEQ ID NO: 20, or the complement thereof. In one aspect, a bGH polyadenylation signal nucleic acid sequence comprises a sequence at least 99.9% identical to SEQ ID NO: 20, or the complement thereof. In one aspect, a bGH polyadenylation signal nucleic acid sequence comprises a sequence 100% identical to SEQ ID NO: 20, or the complement thereof.

As used herein, the term “central nervous system” or “CNS” refers to the brain and spinal cord of a bilaterally symmetric animal. The CNS also includes the retina, the optic nerve, olfactory nerves, and olfactory epithelium.

As used herein, the term “peripheral nervous system” or “PNS” refers to nerves and ganglia outside of the brain and spinal cord, excluding the retina, the optic nerve, olfactory nerves, and olfactory epithelium. In an aspect, the peripheral nervous system is divided into the somatic nervous system and the autonomic nervous system.

As used herein, the term “somatic nervous system” refers to the parts of the PNS that are associated with voluntary control of body movements.

As used herein, the term “autonomic nervous system” refers to the parts of the PNS that regulate the function of internal organs

As used herein, the term “GFAP positive” refers to a cell having detectable protein accumulation of human glial fibrillary acid protein (GFAP) or detectable accumulation of GFAP mRNA expression using techniques standard in the art. In one aspect, a glial cell is GFAP positive.

As used herein, the term “detectable” refers to protein or mRNA accumulation that is identifiable.

Protein accumulation can be identified using antibodies. Non limiting examples of measuring protein accumulation include Western blots, enzyme linked immunosorbent assays (ELISAs), immunoprecipitations and immunofluorescence. An antibody provided herein can be a polyclonal antibody or a monoclonal antibody. An antibody having specific binding affinity for a protein provided herein can be generated using methods well known in the art. An antibody provided herein can be attached to a solid support such as a microtiter plate using methods known in the art.

As used herein, the term “multiplicity of infection” and “MOI” refers to a the number of virions that are added per cell during infection.

As used herein, the term “virion” refers to the infective form of a virus outside a host cell.

As used herein, the term “neurological condition” refers to a disorder, illness, sickness, injury, or disease, in the central nervous system or the peripheral nervous system. Non-limiting examples of neurological conditions can be found in Neurological Disorders: course and treatment, 2^{nd} Edition (2002) (Academic Press Inc.) and Christopher Goetz, Textbook of Clinical Neurology, 3^{rd} Edition (2007) (Saunders).

As used herein, the term “injury” refers to damage to the central nervous system or peripheral nervous system.

In one aspect, a neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis. In one aspect, a neurological condition is Alzheimer's Disease. In one aspect, a neurological condition is Parkinson's Disease. In one aspect, a neurological condition is ALS. In one aspect, a neurological condition is Huntington's Disease. In one aspect, a neurological condition is epilepsy. In one aspect, a neurological condition is a physical injury. In one aspect, a neurological condition is stroke. In one aspect, a neurological condition is ischemic stroke. In one aspect, a neurological condition is hemorrhagic stroke. In one aspect, a neurological condition is cerebral aneurysm. In one aspect, a neurological condition is traumatic brain injury. In one aspect, a neurological condition is concussion. In one aspect, a neurological condition is a tumor. In one aspect, a neurological condition is inflammation. In one aspect, a neurological condition is infection. In, one aspect, a neurological condition is ataxia. In, one aspect, a neurological condition is brain atrophy. In one aspect, a neurological condition is spinal cord atrophy. In one aspect, a neurological condition is multiple sclerosis. In one aspect, a neurological condition is traumatic spinal cord injury. In one aspect, a neurological condition is ischemic or hemorrhagic myelopathy (myelopathy). In one aspect, a neurological condition is global ischemia. In one aspect, a neurological condition is hypoxic ischemic encephalopathy. In one aspect, a neurological condition is embolism. In one aspect, a neurological condition is fibrocartilage embolism myelopathy. In one aspect, a neurological condition is thrombosis. In one aspect, a neurological condition is nephropathy. In one aspect, a neurological condition is chronic inflammatory disease. In one aspect, a neurological condition is meningitis. In one aspect, a neurological condition is cerebral venous sinus thrombosis.

In an aspect, a neurological condition comprises an injury to the CNS or to the PNS. In one aspect, a neurological condition comprises an injury to the CNS. In one aspect, a neurological condition comprises an injury to the PNS.

In an aspect, this disclosure provides, and includes, a method of converting reactive astrocytes to functional neurons in a brain of a living human comprising: injecting an adeno-associated virus (AAV) into a subject in need thereof, where the AAV comprises a DNA vector construct comprising a human distal-less homeobox 2 (hDlx2) sequence comprising the nucleic acid sequence of SEQ ID NO: 6, where the sequence is operably linked to regulatory elements comprising: (a) a human glial fibrillary acid protein (GFAP) promoter comprising a nucleic acid sequence selected from the group consisting of SEQ ID NO: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signal comprising the nucleic acid sequence of SEQ ID NO: 8, a hGH polyadenylation signal comprising that nucleic acid sequence of SEQ ID NO: 13, or a bGH polyadenylation signal comprising that nucleic acid sequence of SEQ ID NO: 20.

In an aspect, this disclosure, and includes, provides a method of converting reactive astrocytes to functional neurons in a brain of a living human comprising: injecting an adeno-associated virus (AAV) into a subject in need thereof, where the AAV comprises a DNA vector construct comprising a nucleic acid coding sequence encoding a human distal-less homeobox 2 (hDlx2) protein comprising the amino acid sequence of SEQ ID NO: 10, where the coding sequence is operably linked to expression control elements comprising: (a) human glial fibrillary acid protein (GFAP) promoter comprising a nucleic acid sequence selected from the group consisting of SEQ ID NOs: 3, 4, and 12; (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; (c) a chimeric intron comprising the nucleic acid sequence selected from the group consisting of SEQ ID NOs: 5 and 19; (d) a woodchuck hepatitis virus posttranscriptional regulatory element (WPRE) comprising the nucleic acid sequence selected from the group consisting of SEQ ID NOs: 7 and 18; and (e) a SV40 polyadenylation signalcomprising the nucleic acid sequence of SEQ ID NO: 8, a hGH polyadenylation signal comprising that nucleic acid sequence of SEQ ID NO: 13, or a bGH polyadenylation signal comprising that nucleic acid sequence of SEQ ID NO: 20.

In an aspect, this disclosure provides, and includes, a method of converting glial cells to neurons in a subject in need thereof comprising: delivering an adeno-associated virus (AAV) to the subject in need thereof, where the AAV comprises a DNA vector construct comprising a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal sequence, where the vector is capable of converting at least one glial cell to a neuron in the subject in need thereof.

In an aspect, this disclosure provides, and includes, a method of treating a neurological condition in a subject in need thereof comprising: delivering an adeno-associated virus (AAV) to the subject, where the AAV comprises a DNA vector construct comprising a distal-less homeobox 2 (Dlx2) sequence operably linked to expression control elements comprising: (a) a glial fibrillary acid protein (GFAP) promoter; (b) an enhancer; (c) a chimeric intron; (d) a woodchuck hepatitis virus posttranscriptional regulatory element (WPRE); and (e) a polyadenylation signal to the subject in need thereof.

In an aspect, a method as provided herein, is capable of converting at least one glial cell to a neuron. In one aspect, a method as provided herein converts at least one glial cell to a neuron.

Neurogenic differentiation 1 (NeuroD 1; also referred to as β2) is a basic helix-loop-helix (bHLH) transcription factor that forms heterodimers with other bHLH proteins to activate transcription of genes that contain a DNA sequence known as an E-box.

Achaete-scute family BHLH transcription factor 1 (Ascl1; also referred to as ASH1, HASH1, MASH-1, and bHLHa46) encodes a member of the basic helix-loop-helix family of transcription factors and is a gene that plays a role in neuronal commitment and differentiation. Insulin gene enhancer protein (ISL1; also known as ISL LIM homeobox-1 and ISLET1) is a gene that encodes a transcription factor containing two N-terminal LIM domains and one C-terminal homeodomain. The encoded protein plays a role in the embryogenesis of pancreatic islets of Langerhans.

LIM-homeobox 3 (LHX3; also known as LIM3 and CPHD3) gene encodes for a protein from a family of proteins with a unique cysteine-rich zinc-binding domain (LIM domain). Huntingtin (Htt; also known as Huntington Disease gene) gene encodes for the huntingtin protein. The wild type contains 6-35 glutamine residues and the mutated Htt contains more than 36 glutamine residue.

In an aspect, a method as provided herein uses an AAV vector comprising a Dlx2 coding sequence in accordance with the present disclosure. In one aspect, a method as provided herein uses an AAV vector comprising a Dlx2 coding sequence in combination with a second AAV vector comprising a second transcription factor coding sequence. In one aspect, a method as provided herein use an AAV vector comprising a Dlx2 coding sequence and a second transcription factor coding sequence. In one aspect, a second transcription factor is selected from the group consisting of NeuroD1, Ascl 1, ISL1, and LHX3. In one aspect, a second transcription factor is NeuroD 1. In one aspect, a second transcription factor is Ascl1. In one aspect, a second transcription factor is ISL1. In one aspect, a second transcription factor is LHX3. In one aspect, a method as provided herein uses an AAV vector comprising a Dlx2 coding sequence and a second Dlx2 coding sequence. In one aspect, a method as provided herein uses an AAV vector comprising a Dlx2 coding sequence in combination with a second AAV vector comprising a Dlx2 coding sequence.

In one aspect, a method as provided herein uses an AAV vector comprising a Dlx2 coding sequence in combination with an AAV vector comprising a shRNA sequence targeting Htt. In an aspect, a method as provided herein uses an AAV vector comprising a Dlx2 coding sequence in combination with an AAV vector comprising an shRNA sequence targeting Htt and a second shRNA sequence targeting Htt. In an aspect, a method as provided herein uses an AAV vector comprising a Dlx2 coding sequence in combination with an AAV vector comprising a shRNA sequence targeting Htt, a second shRNA sequence targeting Htt, and a third shRNA targeting Htt. In an aspect, a method as provided herein uses an AAV vector comprising a Dlx2 coding sequence and a shRNA sequence targeting Htt. In an aspect, a method as provided herein uses an AAV vector comprising a Dlx2 coding sequence and a shRNA sequence targeting Htt and a second shRNA sequence targeting Htt. In an aspect, a method as provided herein uses an AAV vector comprising a Dlx2 coding sequence and a shRNA sequence targeting Htt, a second shRNA sequence targeting Htt and a third shRNA targeting Htt. In an aspect, a method as provided herein uses an AAV vector comprising a shRNA sequence targeting Htt, a second shRNA sequence targeting Htt, and a third shRNA sequence targeting Htt.

In one aspect, a method as provided herein uses an AAV vector comprising a Dlx2 coding sequence in combination with an a ASO sequence targeting Htt. In an aspect, a method as provided herein uses an AAV vector comprising a Dlx2 coding sequence in combination with an ASO sequence targeting Htt and a second ASO sequence targeting Htt. In an aspect, a method as provided herein uses an AAV vector comprising a Dlx2 coding sequence in combination with an ASO sequence targeting Htt, a second ASO sequence targeting Htt, and a third ASO targeting Htt.

In one aspect, a method as provided herein uses an AAV vector comprising a Dlx2 coding sequence in combination with an siRNA sequence targeting Htt. In an aspect, a method as provided herein uses an AAV vector comprising a Dlx2 coding sequence in combination with an siRNA sequence targeting Htt and a second siRNA sequence targeting Htt. In an aspect, a method as provided herein uses an AAV vector comprising a Dlx2 coding sequence in combination with an siRNA sequence targeting Htt, a second siRNA sequence targeting Htt, and a third siRNA targeting Htt.

In one aspect, a method as provided herein uses an AAV vector comprising a Dlx2 coding sequence in combination with an AAV vector comprising a miRNA sequence targeting Htt. In an aspect, a method as provided herein uses an AAV vector comprising a Dlx2 coding sequence in combination with an AAV vector comprising an miRNA sequence targeting Htt and a second miRNA sequence targeting Htt. In an aspect, a method as provided herein uses an AAV vector comprising a Dlx2 coding sequence in combination with an AAV vector comprising a miRNA sequence targeting Htt, a second miRNA sequence targeting Htt, and a third miRNA targeting Htt. In an aspect, a method as provided herein uses an AAV vector comprising a Dlx2 coding sequence and a miRNA sequence targeting Htt. In an aspect, a method as provided herein uses an AAV vector comprising a Dlx2 coding sequence and a miRNA sequence targeting Htt and a second miRNA sequence targeting Htt. In an aspect, a method as provided herein uses an AAV vector comprising a Dlx2 coding sequence and a miRNA sequence targeting Htt, a second miRNA sequence targeting Htt and a third miRNA targeting Htt.

In one aspect, a method as provided herein uses an AAV vector comprising a Dlx2 coding sequence in combination with an AAV vector comprising a gRNA sequence targeting Htt and a CAS nuclease. In an aspect, a method as provided herein uses an AAV vector comprising a Dlx2 coding sequence and a gRNA sequence targeting Htt and a CAS nuclease.

In an aspect, an AAV vector as provided herein, is measured for functionality by assessing transcription levels and protein levels of NeuN, doublecortin (DCX), β3-tubulin, (neurofilament 200) NF-200, (microtubule-associated protein 2) MAP2, ionized calcium binding adaptor molecule (Iba1).

As used herein, the term “NeuN” or “Fox-3” or “Rbfox2” or “Hexaribonucleotide Binding Protein-3” refers to a protein which is a homologue to the protein product of a sex-determining gene in Caenorhabditis elegans and is a neuronal nuclear antigen.

As used herein, the term “DCX” or “doubling” or “lissencephalin-X” refers to a microtubule-associated protein expressed by neuronal precursor cells and immature neurons in embryonic and adult cortical structures.

As used herein, the term “β-tubulin” or “Class III β-tubulin” or “β-tubulin III” refers to a microtubule element of the tubulin family found in neurons.

As used herein, the term “NF-200” refers to a class of protein that is a type IV intermediate filaments found in the cytoplasm of neurons.

As used herein, the term “MAP2” refers to a protein that belongs to the microtubule-associated protein family and play a role in determining and stabilizing neuronal morphology during neuron development.

As used herein, the term “Iba1” refers to a microglia macrophage-specific calcium binding protein.

In an aspect a method provided herein converts glial cells to neurons in combination with gene editing techniques. In one aspect, a gene editing technique targets the mutant Htt. In one aspect, a gene editing technique is selected from the group consisting of siRNA, miRNA, ASO, and CRISPR/CAS. In one aspect, a gene editing technique is siRNA. In one aspect, a gene editing technique is miRNA. In one aspect, a gene editing technique is ASO. In one aspect, a gene editing technique is CRISPR/CAS. In an aspect, a composition as provided herein, is capable of converting at least one glial cell to a neuron. In one aspect, a composition as provided herein converts at least one glial cell to a neuron

As used herein, the term “mammal” refers to any species classified in the class Mammalia.

As used herein, the term “human” refers to a Homo sapiens. In an aspect, a human has a neurological disorder.

As used herein, the term “living human” refers to a human that has heart, respiration and brain activity.

As used herein, the term “non-human primate” refers to any species or subspecies classified in the order Primates that are not Homo sapiens. Non-limiting examples of non-human primates include chimpanzee, bonobo, orangutan, gorilla, macaque, marmoset, capuchin, baboon, gibbon, and lemur.

As used herein, the term “delivering” or “delivery” refers to treating a mammal with an AAV vector or composition as provided herein. In an aspect, an AAV vector or composition as provided herein is delivered to a subject in need thereof. In one aspect, an AAV vector or composition as provided herein is formulated to be delivered to a subject in need thereof. In one aspect, delivering comprises local delivery. In one aspect, an AAV vector or composition as provided herein is formulated for local delivery. In one aspect, delivering comprises systemic delivery. In one aspect, an AAV vector or composition as provided herein is formulated for systemic delivery. In one aspect, delivery comprises injecting an AAV vector or composition as provided herein into a subject in need thereof. In one aspect, delivering is selected from the group consisting of intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration. In one aspect, delivery comprises intraperitoneal delivery. In one aspect, delivery comprises intramuscular delivery. In one aspect, delivery comprises intravenous delivery. In one aspect, delivery comprises intrathecal delivery. In one aspect, delivery comprises intracerebral delivery. In one aspect, delivery comprises intracranial delivery. In one aspect, delivery comprises intra lateral ventricle of the brain delivery. In one aspect, delivery comprises intra cisterna magna delivery. In one aspect, delivery comprises intra vitreous delivery. In one aspect, delivery comprises intra-subretina delivery. In one aspect, delivery comprises intraparenchymal delivery. In one aspect, delivery comprises intranasal delivery. In one aspect, delivery comprises oral administration.

As used herein, the term “injecting” refers to delivering an AAV vector or composition as provided herein under pressure and with force. As a non-limiting example, injecting can comprise the use of a syringe and needle.

In an aspect, an AAV vector or composition as provided herein is injected into a brain of a subject. In one aspect, an AAV vector or composition is injected into a cerebral cortex of a subject. In one aspect, an AAV vector or composition as provided herein is injected in to a spinal cord or a subject. In one aspect, an AAV vector or composition is injected in the striatum of a subject. In one aspect, an AAV vector or composition is injected in the dorsal striatum of a subject. In one aspect, an AAV vector or composition is injected in the putamen of a subject. In one aspect, an AAV vector or composition is injected in the caudate nucleus of a subject. In one aspect, an AAV vector or composition is injected in the substantia nigra of a subject.

In an aspect, an AAV vector or composition as provided herein has spread in the brain between about 1% and about 100%. In one aspect, an AAV vector or composition as provided herein has spread in the brain between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the cerebral cortex between about 1% and about 100%. In one aspect, an AAV vector or composition as provided herein has spread in the cerebral cortex between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the spinal cord between about 1% and about 100%. In one aspect, an AAV vector or composition as provided herein has spread in the spinal cord between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the striatum between about 1% and about 100%. In one aspect, an AAV vector or composition as provided herein has spread in the striatum between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the dorsal striatum between about 1% and about 100%. In one aspect, an AAV vector or composition as provided herein has spread in the dorsal striatum between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the putamen between about 1% and about 100%. In one aspect, an AAV vector or composition as provided herein has spread in the putamen between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the caudate nucleus between about 1% and about 100%. In one aspect, an AAV vector or composition as provided herein has spread in the caudate nucleus between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In and aspect, an AAV vector or composition as provided herein has a spread at from injection site between about 1% and about 100%. In one aspect, an AAV vector or composition as provided herein has a spread from injection site between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

In an aspect, an AAV vector or composition as provided herein has spread in the sub stantia nigra between about 1% and about 100%. In one aspect, an AAV vector or composition as provided herein has spread in the putamen between about 1% and about 10%, between 1% and about 20%, between 1% and about 30%, between 10% and about 20%, between 10% and about 30%, between about 10% and about 40%, between about 20% and about 30%, between about 20% and about 40%, between about 20% and about 50%, between about 30% and about 40%, between about 30% and about 50%, between about 30% and about 60%, between about 40% and about 50%, between about 40% and about 60%, between about 40% and about 70%, between about 50% and about 60%, between about 50% and about 70%, between about 50% and about 80%, between about 60% and about 70%, between about 60% and about 80%, between about 60% and about 90%, between about 70% and about 80%, between about 70% and about 90%, between about 70% and about 100%, between about 80% and about 90%, between about 80% and about 100%, or between about 90% and about 100%.

As used herein, the term “AAV particle” refers to packaged capsid forms of the AAV virus that transmits its nucleic acid genome to cells.

In an aspect, a composition comprising an AAV particle encoded by an AAV vector as provided herein is injected at a concentration between 10¹⁰ AAV particles/mL and 10¹⁴ AAV particles/mL. In one aspect, a composition comprising an AAV particle encoded by an AAV vector as provided herein is injected at a concentration between 10¹⁰ AAV particles/mL and 10¹¹ AAV particles/mL, between 10¹⁰ AAV particles/mL and 10¹² AAV particles/mL, between 10¹⁰ AAV particles/mL and 10¹³ AAV particles/mL, between 10¹¹ AAV particles/mL and 10¹² AAV particles/mL, between 10¹¹ AAV particles/mL and 10¹³ AAV particles/mL, between 10¹¹ AAV particles/mL and 10¹⁴ AAV particles/mL, between 10¹² AAV particles/mL and 10¹³ AAV particles/mL, between 10¹² AAV particles/mL and 10¹⁴ AAV particles/mL, or between 10¹³ AAV particles/mL and 10¹⁴ AAV particles/mL.

In an aspect, a composition comprising an AAV particle encoded by an AAV vector as provided herein is injected at volume between 10 μL, and 1000 μL. In one aspect, a composition comprising an AAV particle encoded by an AAV vector as provided herein is injected at volume between 10 μL and 100 μL, between 10 μL and 200 μL, between 10 μL and 300 μL, between 100 μL and 200 μL, between 100 μL and 300 μL, between 100 μL and 400 μL, between 200 μL and 300 μL, between 200 μL and 400 μL, between 200 μL and 500 μL, between 300 μL and 400 μL, between 300 μL and 500 μL, between 300 μL and 600 μL, between 400 μL and 500 μL, between 400 μL and 600 μL, between 400 μL and 700 μL, between 500 μL and 600 μL, between 500 μL and 700 μL, between 500 μL and 800 μL, between 600 μL and 700 μL, between 600 μL and 800 μL, between 600 μL and 900 μL, between 700 μL and 800 μL, between 700 μL and 900 μL, between 700 μL and 1000 μL, between 800 μL and 900 μL, between 800 μL and 1000 μL, or between 900 μL and 1000 μL.

As used herein, the term “subject” refers to any animal subject. Non-limiting examples of animal subjects include humans, laboratory animals (e.g., primates, rats, mice), livestock (e.g., cows, sheep, goats, pigs, turkeys, chickens), and household pets (e.g., dogs, cats, rodents, etc.).

As used herein, “a subject in need thereof” refers to a subject with a neurological condition. In an aspect, a subject in need thereof has a neurological condition selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis. In one aspect, a subject in need thereof has Alzheimer's Disease. In one aspect, a subject in need thereof has Parkinson's Disease. In one aspect, a subject in need thereof has ALS. In one aspect, a subject in need thereof has Huntington's Disease. In one aspect, a subject in need thereof has epilepsy. In one aspect, a subject in need thereof has a physical injury. In one aspect, a subject in need thereof has stroke. In one aspect, a subject in need thereof has ischemic stroke. In one aspect, a subject in need thereof has hemorrhagic stroke. In one aspect, a subject in need thereof has a cerebral aneurysm. In one aspect, a subject in need thereof has traumatic brain injury. In one aspect, a subject in need thereof has concussion. In one aspect, a subject in need thereof has a tumor. In one aspect, a subject in need thereof has inflammation. In one aspect, a subject in need thereof has an infection. In, one aspect, a subject in need thereof has ataxia. In, one aspect, a subject in need thereof has brain atrophy. In, one aspect, a subject in need thereof has spinal cord atrophy. In one aspect, a subject in need thereof has multiple sclerosis. In one aspect, a subject in need thereof has a traumatic spinal cord injury. In one aspect, a subject in need thereof has ischemic or hemorrhagic myelopathy (myelopathy). In one aspect, a subject in need thereof has global ischemia. In one aspect, a subject in need thereof has hypoxic ischemic encephalopathy. In one aspect, a subject in need thereof has an embolism. In one aspect, a subject in need thereof has fibrocartilage embolism myelopathy. In one aspect, a subject in need thereof has thrombosis. In one aspect, a subject in need thereof has nephropathy. In one aspect, a subject in need thereof has chronic inflammatory disease. In one aspect, a subject in need thereof has meningitis. In one aspect, a subject in need thereof has cerebral venous sinus thrombosis.

In an aspect, a subject in need thereof is a mammal. In one aspect, a subject in need thereof is a human. In one aspect, a subject in need thereof is a non-human primate. In one aspect, a subject in need thereof is selected from the group consisting of chimpanzee, bonobo, orangutan, gorilla, macaque, marmoset, capuchin, baboon, gibbon, and lemur. In one aspect, a subject in need thereof is a chimpanzee. In one aspect, a subject in need thereof is a bonobo. In one aspect, a subject in need thereof is orangutan. In one aspect, a subject in need thereof is gorilla. In one aspect, a subject in need thereof is a macaque. In one aspect, a subject in need thereof is marmoset. In one aspect, a subject in need thereof is a capuchin. In one aspect, a subject in need thereof is a baboon. In one aspect, a subject in need thereof is a gibbon. In one aspect, a subject in need thereof is lemur.

In one aspect, a subject in need thereof is a male. In one aspect, a subject in need thereof is a female. In one aspect, a subject in need thereof is gender neutral. In one aspect, a subject in need thereof is a premature newborn. In one aspect, a premature newborn is born before 36 weeks gestation. In one aspect, a subject in need thereof is a term newborn. In one aspect, a term newborn is below about 2 months old. In one aspect, a subject in need thereof is a neonate. In one aspect, a neonate is below about 1 month old. In one aspect, a subject in need thereof is an infant. In one aspect, an infant is between 2 months and 24 months old. In one aspect, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. In one aspect, a subject in need thereof is a toddler. In one aspect, a toddler is between 1 year and 4 years old. In one aspect, a toddler is between 1 year and 2 years, between 1 year and 3 years, between 1 year and 4 years, between 2 years and 3 years, between 2 years and 4 years, and between 3 years and 4 years old. In one aspect, a subject in need thereof is a young child. In one aspect, a young child is between 2 years and 5 years old. In one aspect, a young child is between 2 years and 3 years, between 2 years and 4 years, between 2 years and 5 years, between 3 years and 4 years, between 3 years and 5 years, and between 4 years and 5 years old. In one aspect, a subject in need thereof is a child. In one aspect, a child is between 6 years and 12 years old. In one aspect, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. In one aspect, a subject in need thereof is an adolescent. In one aspect, an adolescent is between 13 years and 19 years old. In one aspect, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. In one aspect, a subject in need thereof is a pediatric subject. In one aspect, a pediatric subject between 1 day and 18 years old. In one aspect, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. In one aspect, a subject in need thereof is a geriatric subject. In one aspect, a geriatric subject is between 65 years and 95 or more years old. In one aspect, a geriatric subject is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In one aspect, a subject in need thereof is an adult. In one aspect, an adult subject is between 20 years and 95 or more years old. In one aspect, an adult subject is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In one aspect, a subject in need thereof is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. In one aspect, a subject in need thereof is a young old subject (65 to 74 years old). In one aspect, a subject in need thereof is a middle old subject (75 to 84 years old). In one aspect, a subject in need thereof is an old subject (>85 years old).

As used herein, the term “flow rate” refers to the rate of delivery of an AAV vector or composition. In an aspect, the flow rate is between 0.1 μL/minute and 5.0 μL/minute. In one aspect, the flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

As used herein, the term “therapeutically effective dose” or “pharmaceutically active dose” refers to an amount of AAV particles or composition as provided herein which is effective in treating a neurological condition. In an aspect, an AAV particle or composition as provided herein can be provided together with a pharmaceutically acceptable carrier. As used herein, a “pharmaceutically acceptable carrier” refers to a non-toxic solvent, dispersant, excipient, adjuvant, or other material which is mixed with an AAV particles or composition as provided herein.

Non-limiting examples of a pharmaceutically acceptable carrier include a liquid (e.g., saline), gel, nanoparticles, exosomes, lipid vesicles, or solid form of diluents, adjuvant, excipients or an acid resistant encapsulated ingredient. Non-limiting examples of suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In an aspect, a therapeutic effective dose contains auxiliary substances such as wetting or emulsifying agents, stabilizing or pH buffering agents. In one aspect, a therapeutically effective dose of an AAV particle or composition as provided herein is injected to a subject. In one aspect, a therapeutically effective dose of an AAV particle or composition as provided herein is delivered into a subject. In one aspect, a therapeutically effective dose is administered with at least one pharmaceutically acceptable carrier. In one aspect, a therapeutic effective dose contains between about 1% and about 5%, between about 5% and about 10%, between about 10% and about 15%, between about 15% and about 20%, between about 20% and about 25%, between about 25% and about 30%, between about 30% and about 35%, between about 40 and about 45%, between about 50% and about 55%, between about 1% and about 95%, between about 2% and about 95%, between about 5% and about 95%, between about 10% and about 95%, between about 15% and about 95%, between about 20% and about 95%, between about 25% and about 95%, between about 30% and about 95%, between about 35% and about 95%, between about 40% and about 95%, between about 45% and about 95%, between about 50% and about 95%, between about 55% and about 95%, between about 60% and about 95%, between about 65% and about 95%, between about 70% and about 95%, between about 45% and about 95%, between about 80% and about 95%, or between about 85% and about 95% of AAV particle or composition as provided herein.

In an aspect, a therapeutically effective dose is delivered to subject in need thereof at least once daily or at least once weekly for at least two consecutive days or weeks. In one aspect, a therapeutically effective dose is delivered to subject in need thereof at least once daily or at least once weekly for at least 3, 4, 5, 6, 7, 8, 9, 10, 11, 12, 13, 14, or 15 consecutive days or weeks. In one aspect, a therapeutically effective dose is delivered to subject in need thereof at least once daily or at least once weekly for at least 1, 2, 3, 4, 5, 6, 7, 8, 9, 10, 11, or 12 consecutive weeks. In one aspect, a therapeutically effective dose is delivered to subject in need thereof at least once daily or at least once weekly for at most 4, 5, 6, 7, 8, 9, 10, 11, 12, 13, 14, 15, 16, 17, 18, 19, or 20 consecutive days or weeks. In one aspect, a therapeutically effective dose is delivered to subject in need thereof at least once daily or at least once weekly for at most 1, 2, 3, 4, 5, 6, 7, 8, 9, 10, 11, or 12 consecutive weeks or months. In one aspect, a therapeutically effective dose is delivered to subject in need thereof is administered at least once for at least 1, 2, 3, 4, 5, 6, 7, 8, 9, 10, 11, or 12 consecutive months or years, chronically for a subject's entire life span, or an indefinite period of time. In one aspect, a therapeutically effective dose is delivered to subject in need thereof once a year for 2 consecutive years, 3 consecutive years, or 5 consecutive years. In one aspect, a therapeutically effective dose is delivered to subject in need thereof once a year for 2 consecutive years. In one aspect, a therapeutically effective dose is delivered to subject in need thereof once a year for 3 consecutive years. In one aspect, a therapeutically effective dose is delivered to subject in need thereof once a year for 5 consecutive years.

As used herein, the term “remission”, “cure,” or “resolution rate” refers to the percentage of subjects in need thereof that are cured or obtain remission or complete resolution of a neurological condition in response to a therapeutically effective dose.

As used herein, the term “response rate” refers to the percentage of subjects in need thereof that respond positively (e.g., reduced severity or frequency of one or more symptoms) to a therapeutically effective dose.

In an aspect, a therapeutically effective dose achieves a remission, cure, response rate, or resolution rate of a neurological condition of at least about 50%. In one aspect, a therapeutically effective dose eliminates, reduces, slows, or delays, one or more neurological condition symptoms. Non-limiting examples of neurological condition symptoms include tremor, slowed movement (bradykinesia), rigid muscles, impaired posture and balance, loss of automatic movements, speech changes, numbness, and writing changes. In one aspect, a neurological condition symptoms is a movement symptom. Non-limiting examples of movement symptoms include impairment of an involuntary movement or an impairment of a voluntary movement. In one aspect, a neurological condition symptoms is a cognitive symptom. Non-limiting examples of cognitive symptoms include fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information.

In an aspect, neurological condition symptom is a psychiatric symptom. Non-limiting examples of psychiatric symptoms include depression, irritability, sadness or apathy, social withdrawal, insomnia, fatigue, lack of energy, obsessive-compulsive disorder, mania, bipolar disorder, and weight loss. In one aspect, a neurological condition symptom is at least one damaged blood vessel. In one aspect, a neurological condition symptom, is a damaged blood brain barrier (BBB). In one aspect, a neurological condition symptom is damaged blood flow. Non-limiting examples of tests to evaluate the elimination, reduction, slow, or delay, of neurological condition symptoms include the unified Huntington's disease rating scale (UHDRS) score, UHDRS Total Functional Capacity (TFC), UHDRS Functional Assessment, UHDRS Gait score, UHDRS Total Motor Score (TMS), Hamilton depression scale (HAM-D), Columbia-suicide severity rating scale (C-SSRS), Montreal cognitive assessment (MoCA), modified Rankin Scale (mRS), National Institutes of Health Stroke Scale (NIHSS), and Barthel Index (BI), Timed Up and Go Test (TUG), Chedoke Arm and Hand Activity Inventory (CAHAI), Symbol Digit Modalities Test, Controlled Oral Word Association tasks, magnetic resonance imaging (MM), functional magnetic resonance imaging (fMRI), and positron emission tomography (PET) scanning.

In an aspect, a therapeutically effective dose achieves remission, cure, response rate, or resolution rate of a neurological condition of between about 10% and about 99% or more. In one aspect, a therapeutically effective dose achieves remission, cure, response rate, or resolution rate of a neurological condition between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85%and 100%, between 90% and 95%, between 90% and 100%, or between 95% and 100%.

In an aspect, a therapeutically effective dose eliminates, reduces, slows, or delays, one or more neurological condition symptoms between 10% and 100%, such as between 10% to about 15%, between 10% and 20%, between 10% and 25%, between 15% and 20%, between 15% and 25%, between 15% and 30%, between 20% and 25%, between 20% and 30%, between 20% and 35%, between 25 and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In an aspect, a neurological condition symptom is assessed on the day of treatment, 1 day post treatment, 3 months post treatment, 6 months post treatment, 1 year post treatment and every year thereafter post treatment.

In an aspect, a neurological condition symptom is assessed between 1 day post treatment and 7 days post treatment. In one aspect, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In one aspect, symptoms can be assessed between 1 week post treatment and 4 weeks post treatment. In one aspect, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In one aspect, symptoms can be assessed between 1 month post treatment and 12 months post treatment. In one aspect, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In one aspect, symptoms can be assessed between 1 year post treatment and about 20 years post treatment. In one aspect symptoms can be assessed between 1 year post treatment and 5 years post treatment, between 1 year post treatment and 10 years post treatment , between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

As used herein, the term “survival rate” refers to a cohort of subjects in a treatment group still alive after a given period of time after diagnosis of a neurological condition.

In an aspect, a therapeutically effective dose achieves increase survival rate of between about 10% and 99% or more. In one aspect, a therapeutically effective dose achieves an increase in survival rate of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85%and 100%, between 90% and 95%, between 90% and 100%, or between 95% and 100%.

As used herein, the term “life expectancy” refers to a period of time a subject is expected to live.

In an aspect, a therapeutically effective dose increases life expectancy of between about 10% and 99% or more. In one aspect, a therapeutically effective dose increases life expectancy of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85%and 100%, between 90% and 95%, between 90% and 100%, or between 95% and 100%.

In an aspect, a therapeutically effective dose reduces the amount of atrophy within the brain of a subject in need thereof between about 10% and 99% or more. In one aspect, a therapeutically effective dose reduces the amount of atrophy within the brain of a subject in need thereof between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85%and 100%, between 90% and 95%, between 90% and 100%, or between 95% and 100%.

In an aspect, the amount of atrophy within the brain of a subject in need thereof is assessed on the day of treatment, 1 day post treatment, 3 months post treatment, 6 months post treatment, 1 year post treatment and every year thereafter post treatment.

In an aspect, the amount of atrophy within the brain of a subject in need thereof is assessed between 1 day post treatment and 7 days post treatment. In one aspect, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In one aspect, symptoms can be assessed between 1 week post treatment and 4 weeks post treatment. In one aspect, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In one aspect, symptoms can be assessed between 1 month post treatment and 12 months post treatment. In one aspect, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In one aspect, symptoms can be assessed between 1 year post treatment and about 20 years post treatment. In one aspect symptoms can be assessed between 1 year post treatment and 5 years post treatment, between 1 year post treatment and 10 years post treatment , between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

Non-limiting examples of tests to evaluate the amount of atrophy within the brain of a subject in need thereof include Nissle staining, MRI, fMRI, and PET scanning.

While the present disclosure has been described with reference to preferred embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

The examples set out herein illustrate several embodiments of the present disclosure but should not be construed as limiting the scope of the present disclosure in any manner.

### EXAMPLES

### Example 1

### AAV Vector Constructs

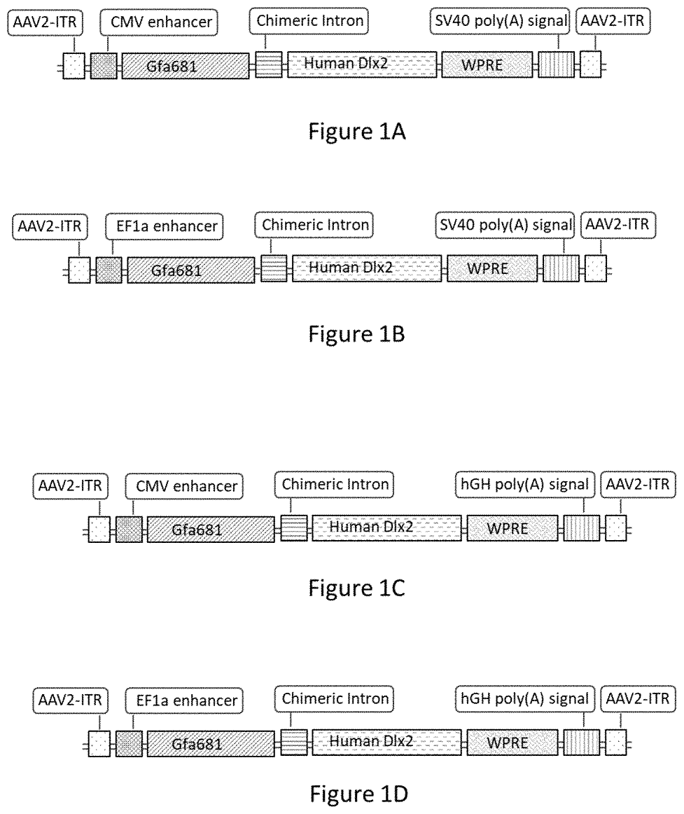

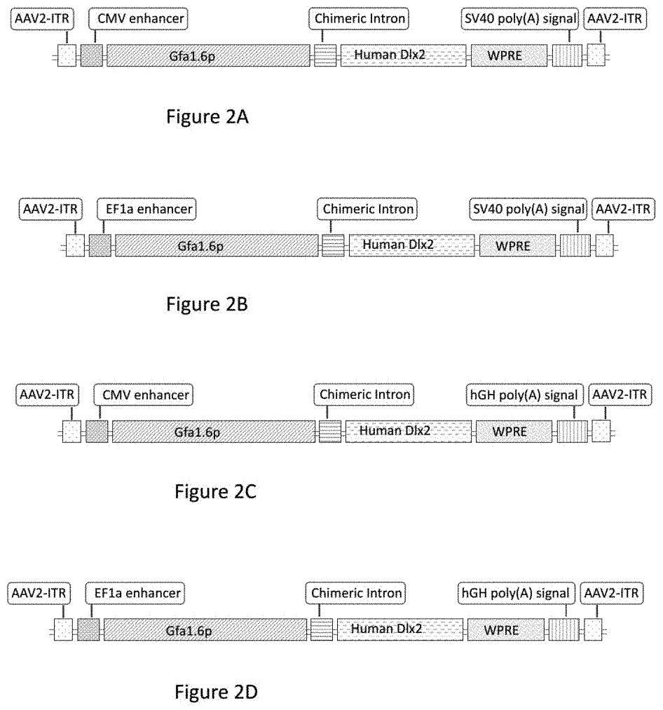

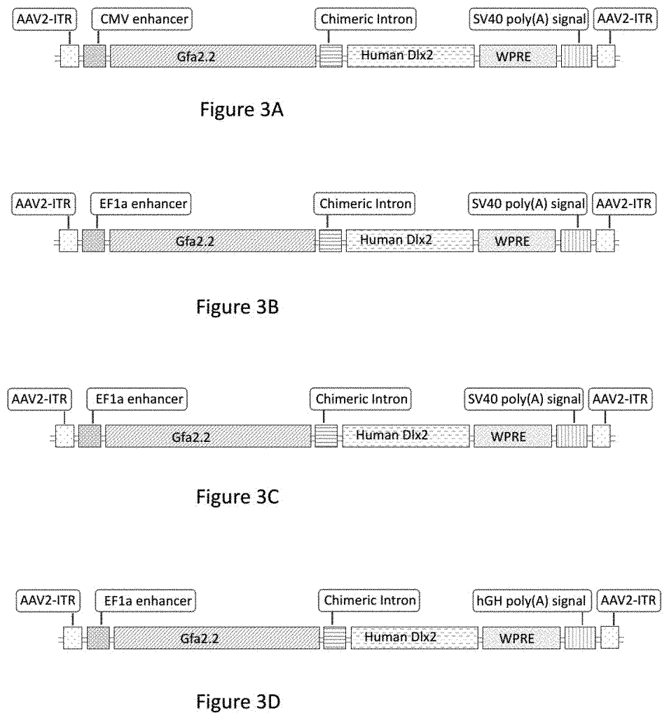

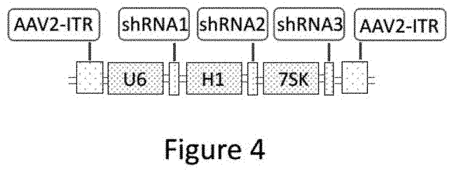

Twelve AAV vectors constructs:
EF-1α:Gfa681:Dlx2:WPRE: SV40 (P44) (FIG. 1B),
EF-1α:Gfa1.6:Dlx2:WPRE:SV40 (FIG. 2B),
EF-1α:GFA2.2:Dlx2:WPRE:SV40 (FIG. 3B),
EF-1α:Gfa681:Dlx2:WPRE:hGH (FIG. 1D),
EF-1α:Gfa1.6:Dlx2:WPRE:hGH (FIG. 2D),
EF-1α:GFA2.2:Dlx2:WPRE:hGh (FIG. 4D),
CE:Gfa681:Dlx2:WPRE:SV40 (P75) (FIG. 1A),
CE:Gfa1.6:Dlx2:WPRE:SV40 (FIG. 2A),
CE:GFA2.2:Dlx2:WPRE:SV40 (FIG. 3A),
CE:Gfa681:Dlx2:WPRE:hGH (FIG. 1C),
CE:Gfa1.6:Dlx2:WPRE:hGH (FIG. 2C),
CE:GFA2.2:Dlx2:WPRE:hGH (FIG. 3D),
EF-1α:Gfa681:Dlx2: WPRE:SV40 (P60) are constructed.

All 12 vector constructs utilize pHSG-299 (Takara, Mountain View, Calif.), a pUC based vector construct which contains an origin of replication, a Kanamycin resistance gene and a multiple cloning site (MSC) with lacZ gene as backbone.

The 5′ end of an expression cassette is an enhancer from a human elongation factor-1 alpha promoter (EF-1 alpha enhancer; SEQ ID NO: 2) or the cytomegalovirus enhancer (CMV enhancer; SEQ ID NO: 11) placed 5′ to either a 758-nucleotide GFAP promoter (Gfa681; SEQ ID NO: 3), a 1667-nucleotide GFAP promoter (Gfa1.6; SEQ ID NO: 4), or a 2214-nucleotide GFAP promoter (GFA2.2 SEQ ID NO: 12).

Following (e.g., 3′ to) the enhancer/GFAP promoter, several additional sequences are introduced into the expression cassette, in 5′ to 3′ direction, including: a chimeric intron (SEQ ID NO: 5 or SEQ ID NO: 19); a human Dlx2 coding sequence (hDlx2; SEQ ID NO: 6); and a woodchuck hepatitis virus posttranscriptional regulatory element (WPRE; SEQ ID NO: 7 or SEQ ID NO: 18). These sequences are all operably linked to an SV40 poly(A) signal (SEQ ID NO: 8) or hGH poly (A) signal (SEQ ID NO: 13). The enhancer, GFAP promoter, chimeric intron, hDlx2 coding sequence, WPRE, and poly(A) signal are flanked by two AAV ITR sequences.

### Example 2

### AAV Virus Production

Each of the twelve plasmids are co-transfected into 293AAV cells using polyethylenimine along with Rep-Cap plasmid (a plasmid comprising a promoter driving the expression of AAV rep and cap genes) and Helper plasmid (a plasmid comprising a promoter driving the expression of E2A, E4, and VA RNA (of Adenovirus) to produce recombinant AAV virus particle. (Cell Biolabs, Inc.)

Transfected cells are scraped and centrifuged at 72 hours after transfection. Cell pellets are frozen and thawed being placed in a dry ice/ethanol mixture followed by being placed in a 37° C. water bath. The freeze/thaw cycle is repeated three additional times. An AAV lysate is purified (e.g., cellular debris is removed) by ultra-centrifugation at 350,00g for 1 hour in discontinuous iodixanol gradients. The virus-containing layer is collected and then concentrated by using Millipore Amicon Ultra Centrifugal Filters. Virus titers are then determined using a by qPCR using primers amplifying ITR regions or gene/expression cassette specific sequences.

### Example 3

### Astrocyte Cell Cultures

Human cortical astrocytes (HA1800; ScienCell Research Laboratories, Inc., Carlsbad, California) are subcultured when they are over 90% confluent. For subculture, cells are trypsinized using TrypLE™ Select (Invitrogen, Carlsbad, Calif.), centrifuged for 5 minutes at 200×g, then resuspended and plated on a medium comprising DMEM/F12 (Gibco); 10% fetal bovine serum (Gibco); penicillin/streptomycin (Gibco); 3.5 mM glucose (Sigma-Aldrich); B27 (Gibco); 10 ng/mL epidermal growth factor (Invitrogen); and 10 ng/mL fibroblast growth factor 2 (Invitrogen). The astrocytes are cultured on poly-D-lysine (Sigma-Aldrich) coated coverslips (12 mm) at a density of approximately 20,000 cells per coverslip in 24-well plates (BD Biosciences).

Rat primary astrocytes (isolated from Sprague Dawley Rat cortex or striatum) are cultured in media comprising DMEM/F12 (Gibco); 10% fetal bovine serum (Gibco), penicillin/streptomycin (Gibco); 3.5 mM glucose (Gibco).

All cells are maintained at 37° C. in humidified air with 5% carbon dioxide.

### Example 4

### Testing AAV Vector in Astrocytes Cell Cultures (In Vitro)

Recombinant AAV obtained from the method of Example 2 are used to infect human cortical astrocytes and rat primary astrocytes of Example 3 at a concentration range of 10¹⁰ particles/mL and 10¹⁴ particles/mL. Twenty-four hours after infection of the cells, the culture medium is replaced by differentiation medium comprising DMEM/F12 (Gibco); N2 supplement (Gibco); and 20 ng/mL brain-derived neurotrophic factor (Invitrogen). The differentiation medium is added to the cell cultures every four days. See Song et al., Nature, 417:39-44 (2002).

Empty space in the cell cultures is filled with additional human astrocytes to support the functional development of converted neurons as astrocytes or rat primary astrocytes convert to neurons.

### Example 5

### Testing of AAV Vector Potency

Recombinant AAV obtained from the method of Example 2 are used to infect human cortical astrocytes and rat primary astrocytes from Example 3 (or astrocytes from other brain regions or the spinal cord) at passage number 4 to 7 at a concentration range of 10¹⁰ particles/mL and 10¹⁴ particles/mL. qPCR, enzyme-linked immunosorbent (ELISA), and western blot are performed to determine expression of Dlx2 transcript and protein levels. Expression of NeuN, doublecortin (DCX), β3-tubulin, NF-200, and MAP2, are assessed by qPCR, ELISA, western blot, and immunostaining to determine functional output of recombinant AAV.

### Example 6

### Testing of AAV Vector Titration and Infection Rate

A purified AAV vector is treated with DNaseI to eliminate remnant plasmid contamination. A series of AAV vector dilutions are performed at 100 times, 500 times , 2500 times, and 12500 times. The AAV plasmid backbone is diluted to generate a standard curve by serial dilutions. The plasmid is diluted 10⁴, 10⁵, 10⁶, 10′, and 10⁸ molecules/μL. qPCR is performed on the diluted AAV vectors and the diluted AAV plasmid. The primers used are against the ITR region (Forward ITR primer, 5′-GGAACCCCTAGTGATGGAGTT, reverse ITR primer, 5′-CGGCCTCAGTGAGCGA). The qPCR mix comprises 10 μL Universal SYBR Master Mix 2×, 2 μL of 5 μM forward ITR primer, 2 μL of 5 μM reverse ITR primer, 5 μL of tested sample or diluted standard and 1 μL H20. The qPCR program is 95 ° C. for 10 minutes followed by 40 cycles of 95 ° C. for 15 seconds, 60 ° C. for 30 seconds followed by a melt curve. The data is analyzed using the qPCR cyclers software. The physical titer of the AAV sample (viral genomes (vg)/ml) is calculated based on the standard curve.

The AAV vector infection rate is tested by using the 50% tissue culture infection dose (TCID50) assay performed using a standard protocol from the American Type Culture Collection (ATCC; Manassas, Va.).

### Example 7

### Testing of AAV Dose Range (In Vivo)

Recombinant AAV obtained from the method of Example 2 is injected into C57/BL6 mice by bilateral intracranial injection into the motor cortex. Each AAV is injected at a dosage of 1×10¹¹, 3×10¹¹, 1×10¹², 3×10¹², 1×10¹², 3×10¹², 1×10¹³ viral genomes/mL at 1 μL of volume. Each dosage is assessed at 4 days, 20 days, and 60 days post injection to determine the optimal effective dose (OED), maximum tolerable dose (MTD), and minimum effective dose (MED) at a cell and tissue level. There are three mice per time point. The OED, MTD, and MED are determine by assessment of astrocyte-to-neuron conversion efficiency and potential toxicity via immunostaining of Dlx2, GFAP, NeuN, and Iba1. If the first dose range is not sufficient to determine the OED, MTD, and MED a second dosage range is performed at 1×10¹⁰ viral genomes/mL to 1×10¹⁴ GC/mL, at 1 μL of volume.

### Example 8

### Testing AAV Vector in Human Subjects (In Vivo)

Recombinant AAV obtained from the method of Example 2 are used to infect human brain or spinal cord astrocytes in vivo. Recombinant AAV is injected at a concentration range of 10¹⁰ particles/mL and 10¹⁴ particles/mL with a volume ranging from 10 μL to 1000 into the brain or spinal cord of a human subject with a neurological condition. The human subject's neurological condition symptoms, brain imaging including Mill, PET scan, or combination of Mill and PET, and behavioral metric's are observed before, during, and post injection. Post injection observations are performed once a week until the first month post injection. After the first month post injection, observations are performed once a month for the next 11 months, and may be extended to 2 years following viral injection.

### Example 9

### Dose Scale Assay in Non-Human Primates

The volume of brain tissue expressing Dlx2 from Example 7 is divided by the number of vector genomes (mm³/vector genomes). The volume (mm³) of specific brain region to be treated in non-human primates is calculated and a dose range of vector genomes is scaled according to the infection rate obtained in Example 7. A dose range study is performed as in Example 7 and the OED, MTD, and MED are determined by assessment of astrocyte-to-neuron conversion efficiency and potential toxicity via immunostaining of Dlx2, GFAP, NeuN, and Iba1.

### Example 10

### Treatment of a Subject in Need Thereof with Huntington's Disease (In Vivo)

A subject with Huntington's Disease is treated with recombinant AAV obtained from the method of Example 2. The subject's neurological symptoms include involuntary movement such as chorea movement, uncontrolled posture, mood change, sleep disorder, speech changes, difficulty with swallowing, and cognitive functions such as deficits in learning and memory. Recombinant AAV is injected at a concentration range of 10¹⁰ particles/mL and 10¹⁴ particles/mL with a volume ranging from 10 μL to 1000 μL into the striatum (putamen and caudate nucleus) of a human subject with a neurological condition. The human subject's neurological condition symptoms, brain imaging including MM, PET scan, or combination of MRI and PET, and behavioral metric's are observed before, during, and post injection. Post injection observations are performed once a week until the first month post injection. After the first month post injection, observations are performed once a month for the next 11 months, and may be extended to 2 years following viral injection.

### Example 11

### A Combination Approach to Directly Converting Glial Cells to Neurons Coupled with shRNA for Knockdown of the Htt Gene Expression

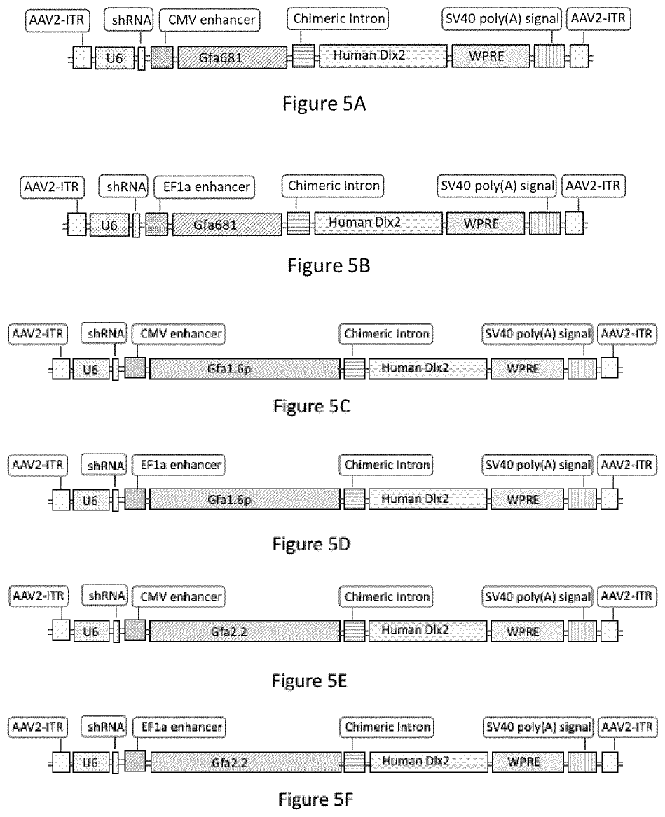

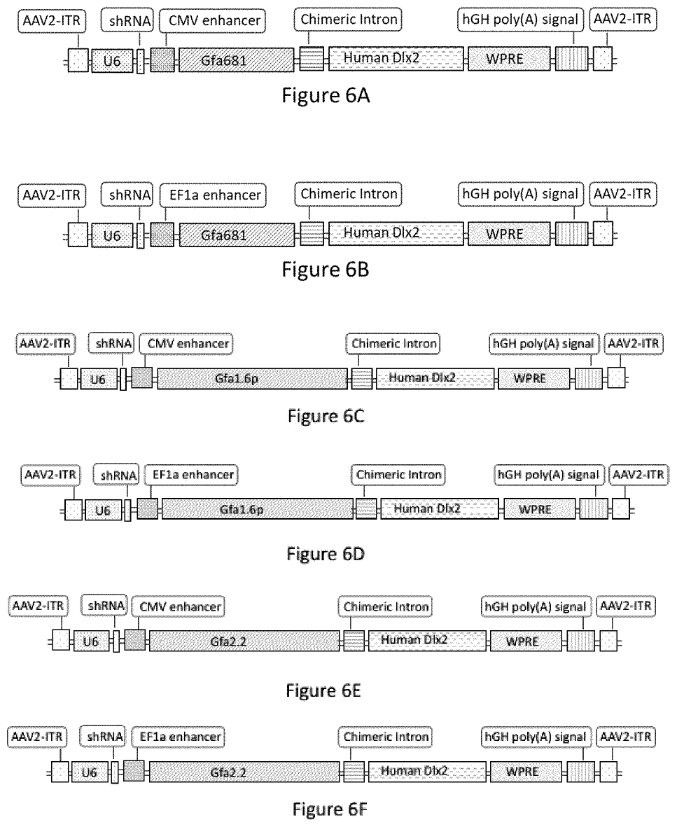

A target sequence is identified that is complementary to the Htt gene. An shRNA is designed to target the Htt gene. Dlx2 and the target shRNA are packaged in to an AAV vector construct (hU6::Htt shRNA-hGFAP::hDlx2) (FIG. 5A-FIG. 6F) and recombinant AAV is produced as described in Example 2. Recombinant AAV is injected into the striatum of mice with mutant Htt gene. Mice receiving the treatment are tested for behavioral metrics, such as cat walk, open field test, clasping, mouse weight, and grip strength and brain imaging including MM, PET scan, or combination of MRI and PET. Behavioral test results and brain imaging are compared among the groups (i) receiving no treatment, (ii) receiving recombinant AAV from Example 2, and (iii) receiving recombinant AAV (hU6::Htt shRNA-hGFAP::hDlx2) (FIG. 5A-FIG. 6F).

Alternatively, the target shRNA are packaged in to an AAV vector construct (hU6::hHtt shRNA) and another recombinant AAV is produced as described in Example 2. The two recombinant AAVs are injected into the striatum of mice with mutant Htt. Mice receiving the treatment are tested for behavioral metrics, such as cat walk, open field test, clasping, mouse weight, and grip strength and brain imaging including MRI, PET scan, or combination of MRI and PET. Behavioral test results and brain imaging are compared among the groups (i) receiving no treatment, (ii) receiving recombinant AAV from Example 2 alone, and (iii) receiving recombinant AAV (hU6::hHtt shRNA) in combination with recombinant AAV from Example 2.

### Example 12

### A Combination Approach to Directly Converting Glial Cells to Neurons Coupled with Crispr/Cas Gene Editing of the Htt Gene

A target sequence is identified that is complementary to the Htt gene. A guide RNA (gRNA) sequence is designed to target the Htt gene. A donor sequence is designed to modify the number of CAG repeats of the Htt gene to less than 36. The Cas9 nuclease, an Htt specific gRNA, and a donor sequence are packaged into AAV vectors construct (AAV-Cas9-HTT). Recombinant AAV is produced as described in Example 2.

Recombinant AAV (AAV-Cas9-Htt) is injected into the striatum of mice with mutant Htt simultaneously with recombinant AAV from Example 2. Mice receiving the treatment are tested for behavioral metrics, such as cat walk, open field test, clasping, mouse weight, and grip strength and brain imaging including MM, PET scan, or combination of MRI and PET. Behavioral test results and brain imaging are compared among the groups (i) receiving no treatment, (ii) receiving recombinant AAV from Example 2, and (iii) receiving recombinant AAV-Cas9-HTT with recombinant AAV from Example 2 to identify synergistic effects between mutant Htt gene editing and glia-to-neuron conversion. Recombinant AAV-Cas9-HTT and recombinant AAV from Example 2 can be injected simultaneously or at different times.

Alternatively, Dlx2, a linker (P2A), a Cas9 nuclease, an Htt specific gRNA and a donor sequence are packaged into AAV vectors construct (AAV-hDlx2-P2A-Cas9-HTT). Recombinant AAV is produced as described in Example 2. Recombinant AAV (AAV-hDlx2-P2A-Cas9-HTT) is injected into the striatum of mice with mutant Htt simultaneously with recombinant AAV from Example 2. Mice receiving the treatment are tested for behavioral metrics, such as cat walk, open field test, clasping, mouse weight, and grip strength and brain imaging including MRI, PET scan, or combination of MRI and PET. Behavioral test results and brain imaging are compared among the groups (i) receiving no treatment, (ii) receiving recombinant AAV from Example 2, and (iii) receiving recombinant AAV-hDlx2-P2A-Cas9-HTT with recombinant AAV from Example 2 to identify synergistic effects between mutant Htt gene editing and glia-to-neuron conversion.

### Example 13

### A Combination Approach to Directly Converting Glial Cells to Neurons Coupled with Antisense Oligonucleotide (ASO) to Knock Down the Htt Gene Expression

A target sequence is identified that is complementary to the Htt gene. An ASO is designed and synthesized to knock down the Htt gene expression. Recombinant AAV from Example 2 is injected together with Htt ASO into the striatum of mice with mutant Htt. Mice receiving the treatment are tested for behavioral metrics, such as cat walk, open field test, clasping, mouse weight, and grip strength and brain imaging including MM, PET scan, or combination of MRI and PET. Behavioral test results and brain imaging are compared among the groups (i) receiving no treatment, (ii) receiving recombinant AAV from Example 2, and (iii) receiving recombinant AAV from Example 2 together with Htt ASO.

### Example 14

### A Combination Approach to Directly Converting Glial Cells to Neurons Coupled with Sirna to Knock Down the Htt Gene Expression

A target sequence is identified that is complementary to the Htt gene. An siRNA is designed and synthesized to knock down the Htt gene expression. Recombinant AAV from example 2 is injected together with Htt siRNA into the striatum of mice with mutant Htt. Mice receiving the treatment are tested for behavioral metrics, such as cat walk, open field test, clasping, mouse weight, and grip strength and brain imaging including MM, PET scan, or combination of MRI and PET. Behavioral test results and brain imaging are compared among the groups (i) receiving no treatment, (ii) receiving recombinant AAV from Example 2, and (iii) receiving recombinant AAV from Example 2 together with Htt siRNA.

### Example 15

### A Combination Approach to Directly Converting Glial Cells to Neurons Coupled with Mirna to Knock Down the Htt Gene Expression

A miRNA is identified that is regulating the Htt gene expression. NeuroD1, Dlx2, and the miRNA are packaged into an AAV vector (CAG::Htt miRNA-hGFAP::hDlx2) and recombinant AAV is produced as described in Example 2. Recombinant AAV is injected into the striatum of mice with mutant Htt. Mice receiving the treatment are tested for behavioral metrics, such as cat walk, open field test, clasping, mouse weight, and grip strength and brain imaging including MRI, PET scan, or combination of MRI and PET. Behavioral test results and brain imaging are compared among the groups (i) receiving no treatment, (ii) receiving recombinant AAV from Example 2, and (iii) receiving recombinant AAV (CAG::Htt miRNA-hGFAP::hDlx2).

Alternatively, the target miRNA is packaged in to an AAV vector (CAG::hHtt miRNA) and recombinant AAV is produced as described in Example 2. Recombinant AAV is injected into the striatum of mice with mutant Htt. Mice receiving the treatment are tested for behavioral metrics, such as cat walk, open field test, clasping, mouse weight, and grip strength and brain imaging including MRI, PET scan, or combination of MRI and PET. Behavioral test results and brain imaging are compared among the groups (i) receiving no treatment, (ii) receiving recombinant AAV from Example 2 alone, and (iii) receiving recombinant AAV (CAG::hHtt miRNA) in combination with recombinant AAV from Example 2.

### Example 16

### Successful Establishment of Rat Astrocytes Primary Culture

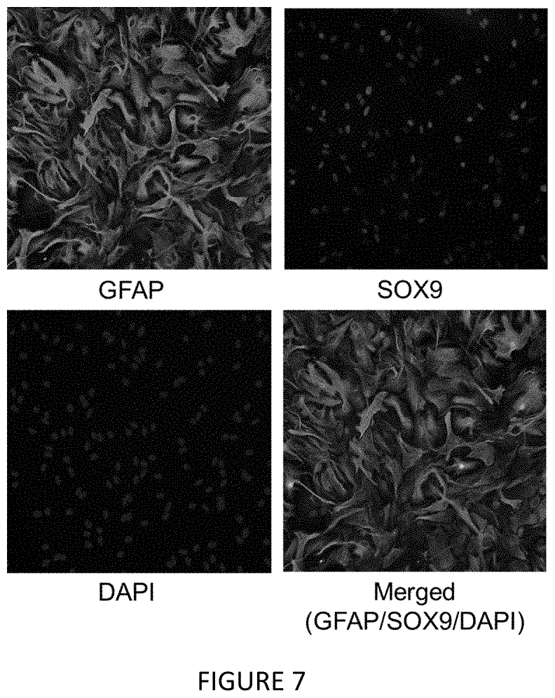

Cortical and striatum tissue is isolated from 3 day post-natal Sprague-Dawley rat brains. Tissue is treated with papain to generate single cell suspension and seeded in flasks coated with poly-D-lysine. Cells are immunostained with GFAP antibody and SOX9 antibody. Cells are counter stained with DAPI antibody. More than 95% of cells (at passage 6) are astrocytes identified by GFAP and SOX9 staining (FIG. 7). Far left panel presents an image of GFAP stained cells. Middle left panel presents an image of SOX9 stained cells. Middle right panel presents an image of DAPI stained cells. Far right panel presents a merge image of GFAP, SOX9, and DAPI stained cells.

### Example 17

### Dlx2 Expression in Astrocyte Transfected with Plasmids

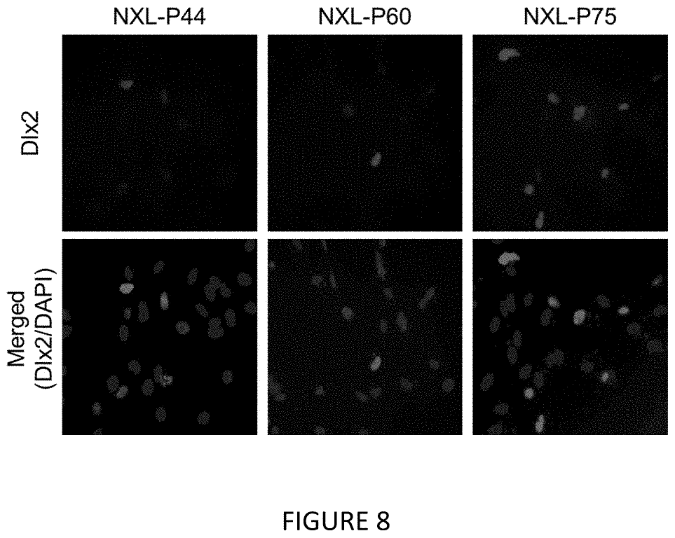

Primary rat astrocytes are seeded and transfected as described in Example 2 with expression vectors P44 (EE:pGfa681:CI:Dlx2:WPRE:SV40), P60 (EE:pGfa681:Dlx2:WPRE: SV40), P75 (CE:pGfa681:CI:Dlx2:WPRE: SV40). Dlx2 protein expression is visualized by immunostaining with anti-Dlx2 antibody followed by fluorescent dye conjugated secondary antibody and image captured using fluorescent microscope. (FIG. 8; top panels show Dlx2 staining of cells, bottom panels show merged Dlx2 and DAPI staining of cells).

### Example 18

### Quantitative Analysis of Transduction of AAV Virus Particles into Primary Rat Astrocytes

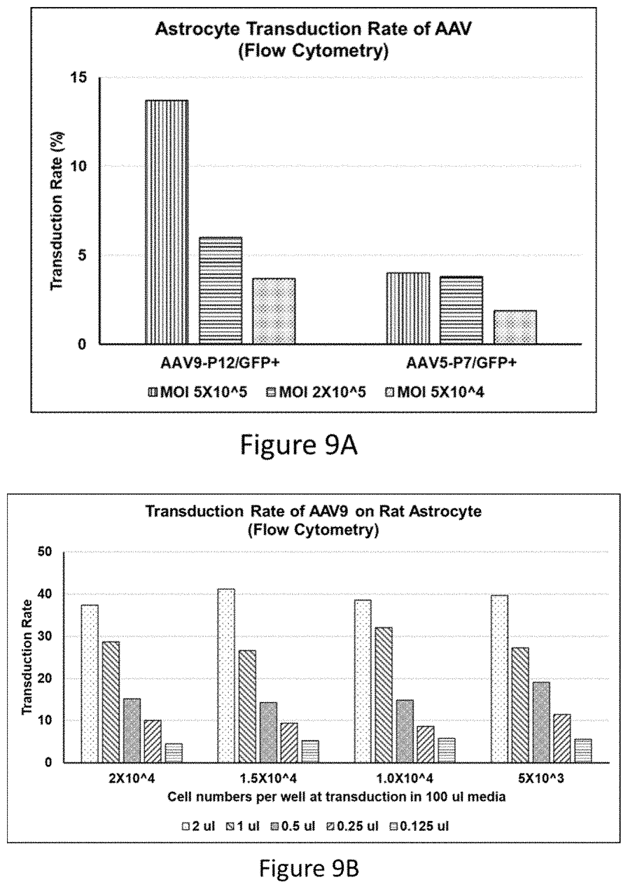

Recombinant AAV obtained from the method of Example 2 is transduced into primary rat astrocytes seeded in 24-well plates or 96-well plates with viral particles AAV9-P12 (pGfa681:GFP) and AAVS-P7 (pEF-1α:GFP). Cells are harvested seven days post-infection by trypsinization. The cells are fixed, washed, and suspended in PBS. The viral transduction rate is analyzed using flow cytometry to count GFP positive cells compared with all cells (FIG. 9A-9B). FIG. 9A shows the percentage transduction rate of AAV9-P12 (pGfa681:GFP) and AAV5-P7 (pEF-1α: GFP) at MOIs of 5×10⁵ vg/cell, 2×10⁵ vg/cell, and 5×10⁴ vg/cell. FIG. 9B shows the transduction rate of AAV viral particles in cells seeded in 96 well plates at a series of densities of 2×10⁴ cell/well, 1.5×10⁴ cell/well, 1×10⁴ cell/well, and 5×10³ cell/well, and infected with virus at a series of amounts of 2 μl, 1 μl, 0.5 0.25 0.125 μl of 1×10¹³ vg/ml virus in 100 μl of medium.

### Example 19

### In Vitro Transgene Expression of Dlx2

Vectors: The vectors are tested via transfection of rat cortical astrocytes (RACs). Additionally, AAVs are produced with selected vectors and tested in vitro via transduction: NXL-P44: EE-pGfa681-CI-Dlx2-WPRE-SV40pA
NXL-P60: EE-pGfa681-Dlx2-WPRE-SV40pA
NXL-P75: CE-pGfa681-CI-Dlx2-WPRE-SV40pA
NXL-P104: CE-pGfa681-CGRI-Dlx2-bGHpA 1vNXL-P105: CE-pGfa681-CI-Dlx2-oPRE-bGHpA
NXL-P131: EE-pGfa681-CI-Dlx2-oPRE-bGHpA
NXL-P133: CE-pGfa681-CGRI-Dlx2-oPRE-bGHpA
NXL-P137: EE-pGfa681-CGRI-Dlx2-oPRE-bGHpA

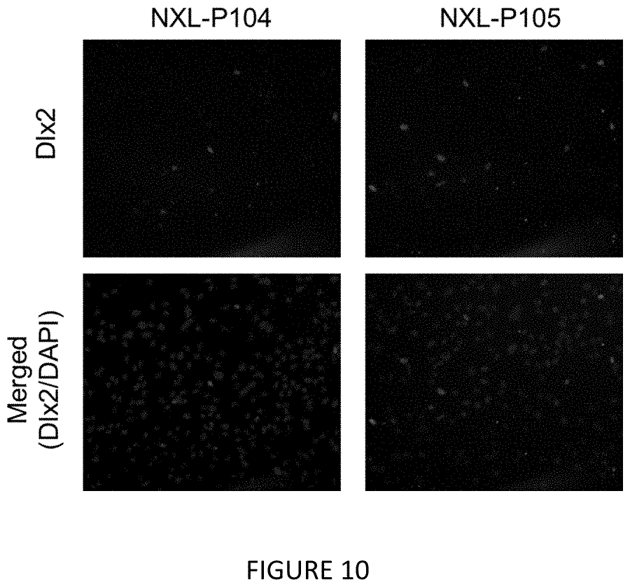

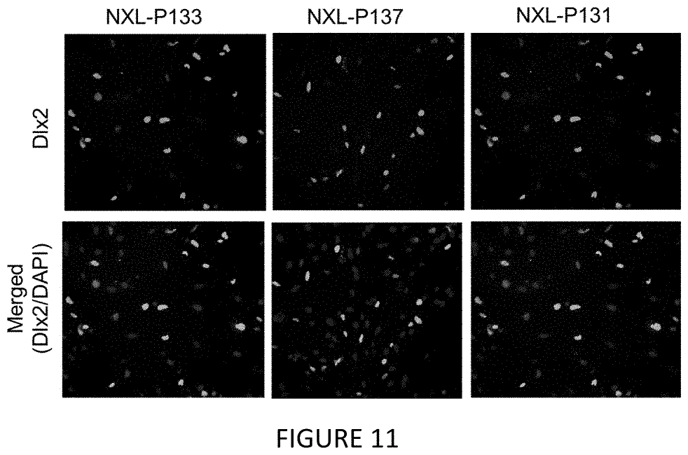

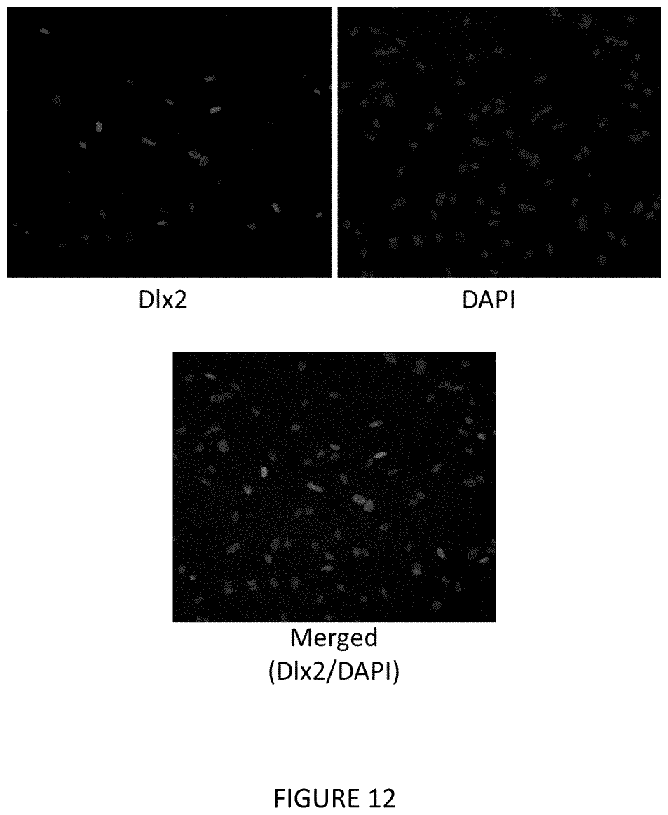

NXL-P104 and NXL-P105 constructs are effective in driving the expression of Dlx2 24 hours post transfection of the cultured RACs, as demonstrated by the positive Dlx2 staining in these cells (FIG. 10). NXL-P133, NXL-P137, and NXL-P131 constructs are effective in driving the expression of Dlx2 24 hours post transfection of the cultured RACs, as demonstrated by the positive Dlx2 staining in these cells (FIG. 11). AAV9-P133 (the AAV produced with NLX-P133) is effective in driving the expression of Dlx2 after transducing cultured RACs with this virus, as demonstrated by the positive Dlx2 staining in these cells (FIG. 12).

A variety of further modifications and improvements in and to the compositions and methods of the present disclosure will be apparent to those skilled in the art based. The following non-limiting embodiments are envisioned:
1. An adeno-associated virus (AAV) vector comprising a human distal-less homeobox 2 (hDlx2) sequence comprising the nucleic acid sequence of SEQ ID NO: 6, wherein the hDlx2 sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
2. An adeno-associated virus (AAV) vector comprising a nucleic acid coding sequence encoding a human distal-less homeobox 2 (hDlx2) protein comprising the amino acid sequence of SEQ ID NO: 10, wherein said coding sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal with a nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
3. An adeno-associated virus (AAV) vector comprising a distal-less homeobox 2 (Dlx2) nucleic acid coding sequence encoding a Dlx2 protein, wherein said coding sequence is operably linked to regulatory elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal sequence.
4. A composition comprising an adeno-associated virus (AAV) vector for converting glial cells to functional neurons in a human, wherein said AAV vector comprises a human distal-less homeobox 2 (hDlx2) sequence having a nucleic acid sequence of SEQ ID NO: 6, and wherein said sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
5. A composition comprising an adeno-associated-virus (AAV) vector for converting glial cells to functional neurons in a human, wherein said AAV vector comprises a nucleic acid sequence encoding a distal-less homeobox 2 (hDlx2) protein comprising the amino acid coding sequence of SEQ ID NO: 10, and wherein said coding sequence is operably linked to regulatory elements comprising:
   (a) a human glial fibrillary acidic protein (GFAP) promoter comprising a nucleic acid sequence selected from the group consisting of SEQ ID NOs: 3,4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal sequence comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
6. A composition comprising an adeno-associated virus (AAV) vector for the treatment of a subject in need thereof, wherein said AAV vector comprises a distal-less homeobox 2 (Dlx2) sequence operably linked to expression control elements comprising:
   (a) a glial fibrillary acidic protein (GFAP) promoter;
   (b) an enhancer;
   (c) a chimeric intron;
   (d) a woodchuck hepatitis virus posttranscriptional regulatory element (WPRE); and
   (e) a polyadenylation signal.
7. The AAV vector of any one of embodiments 1-3, or the composition of any one of embodiments 4-6, wherein said AAV vector is selected from the group consisting of AAV serotype 2, AAV serotype 5, and AAV serotype 9.
8. The AAV vector or composition of embodiment 7, wherein said AAV vector is AAV serotype 2.
9. The AAV vector or composition of embodiment 7, wherein said AAV vector is AAV serotype 5.
10. The AAV vector or composition of embodiment 7, wherein said AAV vector is AAV serotype 9.
11. The composition of embodiment 4 or 5, wherein said glial cells are reactive astrocytes.
12. The composition of embodiment 4 or 5, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
13. The composition of embodiment 4 or 5, wherein said human has a neurological condition.
14. The AAV vector of embodiment 3, or the composition of embodiment 6, wherein said Dlx2 is a human Dlx2 (hDlx2).
15. The AAV vector of embodiment 3, or the composition of embodiment 6, wherein said Dlx2 is selected from the group consisting of a chimpanzee Dlx2, a bonobo Dlx2, an orangutan Dlx2, a gorilla Dlx2, a macaque Dlx2, a marmoset Dlx2, a capuchin Dlx2, a baboon Dlx2, a gibbon Dlx2, and a lemur Dlx2.
16. The AAV vector or composition of embodiment 14, wherein said hDlx2 comprises a nucleic acid coding sequence encoding an amino acid sequence at least 80% identical or similar to SEQ ID NO: 10.
17. The AAV vector or composition of embodiment 14, wherein said hDlx2 coding sequence comprises a nucleic acid sequence at least 80% identical to SEQ ID NO: 6, or the complement thereof.
18. The AAV vector of embodiment 3, or the composition of embodiment 6, wherein said GFAP promoter is a human GFAP (hGFAP) promoter.
19. The AAV vector of embodiment 3, or the composition of embodiment 6, wherein said GFAP promoter is selected from the group consisting of a chimpanzee GFAP promoter, a bonobo GFAP promoter, an orangutan GFAP promoter, a gorilla GFAP promoter, a macaque GFAP promoter, a marmoset GFAP promoter, a capuchin GFAP promoter, a baboon GFAP promoter, a gibbon GFAP promoter, and a lemur GFAP promoter.
20. The AAV vector or composition of embodiment 18, wherein said hGFAP promoter comprises a nucleic acid sequence at least 80% identical to SEQ ID NO: 3, or the complement thereof.
21. The AAV vector or composition of embodiment 18, wherein said hGFAP promoter comprises a nucleic acid sequence at least 80% identical to SEQ ID NOs: 4 or the complement thereof.
22. The AAV vector or composition of embodiment 18, wherein said hGFAP promoter comprises a nucleic acid sequence at least 80% identical to SEQ ID NOs: 12 or the complement thereof.
23. The AAV vector of embodiment 3, or the composition of embodiment 6, wherein said enhancer is selected from the group consisting of an enhancer from human elongation factor-1 alpha (EF1-α) promoter and cytomegalovirus (CMV) enhancer.
24. The AAV vector or composition of embodiment 23, wherein said EF1-α comprises a nucleic acid sequence at least 80% identical to SEQ ID NO: 2, or the complement thereof.
25. The AAV vector or composition of embodiment 23, wherein said CMV enhancer comprises a nucleic acid sequence at least 80% identical to SEQ ID NO: 11, or the complement thereof.
26. The AAV vector of embodiment 3, or the composition of embodiment 6, wherein said chimeric intron comprises a nucleic acid sequence at least 80% identical to a nucleic acid selected from the group consisting of SEQ ID NOs: 5 and 19, or the complement thereof.
27. The AAV vector of embodiment 3, or the composition of embodiment 6, wherein said WPRE comprises a nucleic acid sequence at least 80% identical to a nucleic acid selected from the group consisting of SEQ ID NOs: 7 and 18, or the complement thereof.
28. The AAV vector of embodiment 3, or the composition of embodiment 6, wherein said polyadenylated signal is selected from the group consisting of SV40 polyadenylation signal a hGH polyadenylation signal, and a bGH polyadenylation signal.
29. The AAV vector or composition of embodiment 28, wherein said SV40 polyadenylated signal comprises a nucleic acid sequence at least 80% identical to SEQ ID NO: 8, or the complement thereof.
30. The AAV vector or composition of embodiment 28, wherein said hGH polyadenylated signal comprises a nucleic acid sequence at least 80% identical to SEQ ID NO: 13, or the complement thereof.
31. The AAV vector or composition of embodiment 28, wherein said bGH polyadenylated signal comprises a nucleic acid sequence at least 80% identical to SEQ ID NO: 20, or the complement thereof.
32. The AAV vector of embodiment 3, or the composition of embodiment 6, wherein said AAV vector further comprises a nucleic acid sequence encoding an AAV protein sequence.
33. The AAV vector of any one of embodiments 1-3, or the composition of any one of embodiments 4-6, wherein said AAV vector comprises AAV serotype 2 inverted terminal repeats (ITRs).
34. The AAV vector of any one of embodiments 1-3, or the composition of any one of embodiments 4-6, wherein said AAV vector comprises AAV serotype 5 inverted terminal repeats (ITRs).
35. The AAV vector of any one of embodiments 1-3, or the composition of any one of embodiments 4-6, wherein said AAV vector comprises AAV serotype 9 inverted terminal repeats (ITRs).
36. The AAV vector of any one of embodiments 1-3, or the composition of any one of embodiments 4-6, wherein said AAV vector comprises at least one ITR nucleic acid sequence at least 80% identical to SEQ ID NO: 1.
37. The AAV vector of any one of embodiments 1-3, or the composition of any one of embodiments 4-6, wherein said AAV vector comprises at least one ITR nucleic acid sequence at least 80% identical to SEQ ID NO: 9.
38. The composition of embodiment 6, wherein said subject in need thereof is a mammal.
39. The composition of embodiment 38, wherein said mammal is a human.
40. The composition of embodiment 38, wherein said mammal is a non-human primate.
41. The composition of embodiment 6, wherein said subject in need thereof has a neurological condition.
42. The composition of embodiment 13 or 41, wherein said neurological condition comprises an injury to the central nervous system (CNS) or peripheral nervous system.
43. The composition of embodiment 13 or 41, wherein said wherein said neurological condition comprises an injury to the CNS.
44. The composition of embodiment 13 or 41, wherein said neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.
45. The composition of embodiment 13 or 41, wherein said neurological condition is Alzheimer's Disease.
46. The composition of embodiment 13 or 41, wherein said neurological condition is Parkinson's Disease.
47. The composition of embodiment 13 or 41, wherein said neurological condition is ALS.
48. The composition of embodiment 13 or 41, wherein said neurological condition is Huntington's Disease.
49. The composition of embodiment 13 or 41, wherein said neurological condition is a stroke.
50. The composition of embodiment 49, wherein said stroke is an ischemic stroke.
51. The composition of embodiment 49, wherein said stroke is a hemorrhagic stroke.
52. The composition of embodiment 41, wherein said composition is capable of converting at least one glial cell to a neuron.
53. The composition of embodiment 52, wherein said glial cells are selected from the group consisting of astrocytes and NG2 cells.
54. The composition of embodiment 52, wherein said glial cells are astrocytes.
55. The composition of embodiment 54, wherein said astrocytes are reactive astrocytes.
56. The composition of embodiment 52, wherein said glial cells are GFAP positive.
57. The composition of embodiment 52, wherein said neurons are functional neurons.
58. The composition of embodiment 52, wherein said functional neurons are selected from the group consisting of glutamatergic neurons, GABAergic neurons. dopaminergic neurons, cholinergic neurons, seratonergic neurons, epinephrinergic neurons, motor neurons, and peptidergic neurons.
59. The composition of embodiment 58, wherein said functional neurons are glutamatergic neurons.
60. The composition of embodiment 6, wherein said composition is formulated to be delivered to a subject in need thereof.
61. The composition of embodiment 60, wherein said composition is formulated for local delivery.
62. The composition of embodiment 61, wherein said composition is formulated for systemic delivery.
63. The composition of any one of embodiments 60-62, wherein said composition is formulated for delivery via intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration.
64. A method comprising delivering the composition of embodiment 6 to said subject in need thereof.
65. The method of embodiment 64, wherein said composition is formulated to be delivered to a subject in need thereof.
66. The method of embodiment 64, wherein said delivering comprises local administration.
67. The method of embodiment 64, wherein said delivering comprises systemic administration.
68. The method of any one of embodiments 64-67, wherein said delivering comprises an intraperitoneal, intramuscular, intravenous, intrathecal, intracerebral, intracranial, intra lateral ventricle of the brain, intra cisterna magna, intra vitreous, intra-subretina, intraparenchymal, intranasal, or oral administration.
69. A method of converting reactive astrocytes to functional neurons in a brain of a living human comprising: injecting an adeno-associated virus (AAV) into a subject in need thereof, wherein said AAV comprises a DNA vector construct comprising a human distal-less homeobox 2 (hDlx2) sequence comprising the nucleic acid sequence of SEQ ID NO: 6, wherein said sequence is operably linked to regulatory elements comprising:
   (a) a human glial fibrillary acid protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
70. A method of converting reactive astrocytes to functional neurons in a brain of a living brain comprising: injecting an adeno-associated virus (AAV) into a subject in need thereof, wherein said AAV comprises a DNA vector construct comprising a nucleic acid sequence encoding a human distal-less homeobox 2 (hDlx2) protein comprising the amino acid coding sequence of SEQ ID NO: 10, wherein said coding sequence is operably linked to expression control elements comprising:
   (a) a human glial fibrillary acid protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11;
   (c) a chimeric intron comprising the nucleic acid sequence selected from the group consisting of SEQ ID NOs: 5 and 19;
   (d) a woodchuck hepatitis virus posttranscriptional regulatory element (WPRE) comprising the nucleic acid sequence selected from the group consisting of SEQ ID NOs: 7 and 18; and
   (e) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
71. A method of converting glial cells to neurons in a subject in need thereof comprising: delivering an adeno-associated virus (AAV) to said subject in need thereof, wherein said AAV comprises a DNA vector construct comprising a distal-less homeobox 2 (Dlx2) sequence operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) and a polyadenylation signal sequence,
wherein said vector is capable of converting at least one glial cell to a neuron in said subject in need thereof.
72. A method of treating a neurological condition in a subject in need thereof comprising: delivering an adeno-associated virus (AAV) to said subject, wherein said AAV comprises a DNA vector construct comprising a distal-less homeobox 2 (Dlx2) sequence operably linked to expression control elements comprising:
   (a) a glial fibrillary acid protein (GFAP) promoter;
   (b) an enhancer;
   (c) a chimeric intron;
   (d) a woodchuck hepatitis virus posttranscriptional regulatory element (WPRE); and
   (e) a polyadenylation signal to said subject in need thereof.
73. The method of any one of embodiments 69-72, wherein said AAV is selected from the group consisting of AAV serotype 2, AAV serotype 5, and AAV serotype 9.
74. The method of embodiment 73, wherein said AAV is AAV serotype 2.
75. The method of embodiment 73, wherein said AAV is AAV serotype 5.
76. The method of embodiment 73, wherein said AAV is AAV serotype 9.
77. The method of embodiments 69 or 70, wherein said functional neurons are glutamatergic neurons, GABAergic neurons, dopaminergic neurons, cholinergic neurons, seratonergic neurons, epinephrinergic neurons, motor neurons, and peptidergic neurons.
78. The method of embodiments 71 or 72, wherein said Dlx2 is human Dlx2 (hDlx2).
79. The method of embodiments 71 or 72, wherein said Dlx2 is selected from the group consisting of a chimpanzee Dlx2, a bonobo Dlx2, an orangutan Dlx2, a gorilla Dlx2, a macaque Dlx2, a marmoset Dlx2, a capuchin Dlx2, a baboon Dlx2, a gibbon Dlx2, and a lemur Dlx2.
80. The method of embodiment 78, wherein said hDlx2 comprises a amino acid sequence encoding an amino acid sequence at least 80% identical or similar to SEQ ID NO: 10.
81. The method of embodiment 78, wherein said hDlx2 coding sequence comprises a nucleic acid sequence at least 80% identical to SEQ ID NO: 6, or the complement thereof.
82. The method of embodiments 71 or 72, wherein said GFAP promoter is a human GFAP (hGFAP) promoter.
83. The method of embodiments 71 or 72, wherein said GFAP promoter is selected from the group consisting of a chimpanzee GFAP promoter, a bonobo GFAP promoter, an orangutan GFAP promoter, a gorilla GFAP promoter, a macaque GFAP promoter, a marmoset GFAP promoter, a capuchin GFAP promoter, a baboon GFAP promoter, a gibbon GFAP promoter, and a lemur GFAP promoter.
84. The method of embodiment 82, wherein said hGFAP promoter comprises a nucleic acid sequence at least 80% identical to SEQ ID NO: 3, or the complement thereof.
85. The method of embodiment 83, wherein said hGFAP promoter comprises a nucleic acid sequence at least 80% identical to SEQ ID NOs: 4, or the complement thereof.
86. The method of embodiment 82, wherein said hGFAP promoter comprises a nucleic acid sequence at least 80% identical to SEQ ID NOs: 12, or the complement thereof.
87. The method of embodiments 71 or 72, wherein said enhancer is selected from the group consisting of an enhancer from human elongation factor-1 alpha (EF1-α) promoter and cytomegalovirus (CMV) enhancer.
88. The method of embodiment 87, wherein said EF1-α comprises a nucleic acid sequence at least 80% identical to SEQ ID NO: 2, or the complement thereof.
89. The method of embodiment 87 wherein said CMV enhancer comprises a nucleic acid sequence at least 80% identical to SEQ ID NO: 11, or the complement thereof.
90. The method of embodiments 71 or 72, wherein said chimeric intron comprises a nucleic acid sequence at least 80% identical to nucleic acid sequence selected from the group consisting of SEQ ID NOs: 5 and 19, or the complement thereof.
91. The method of embodiments 71 or 72, wherein said WPRE comprises a nucleic acid sequence at least 80% identical to a nucleic acid sequence selected from the group consisting of SEQ ID NOs: 7 and 18, or the complement thereof.
92. The method of embodiments 71 or 72, wherein said polyadenylated signal is selected from the group consisting of SV40 polyadenylation signal and a hGH polyadenylation signal.
93. The method of embodiments 71 or 72, wherein said SV40 polyadenylated signal comprises a nucleic acid sequence at least 80% identical to SEQ ID NO: 8, or the complement thereof.
94. The method of embodiments 71 or 72, wherein said hGH polyadenylated signal comprises a nucleic acid sequence at least 80% identical to SEQ ID NO: 13, or the complement thereof.
95. The method of embodiments 71 or 72, wherein said bGH polyadenylated signal comprises a nucleic acid sequence at least 80% identical to SEQ ID NO: 20, or the complement thereof.
96. The method of embodiments 71 or 72, wherein said vector further comprises a nucleic acid sequence encoding an AAV protein sequence.
97. The method of any one of embodiments 69-72, wherein said vector comprises AAV serotype 2 inverted terminal repeats (ITRs).
98. The method of any one of embodiments 69-72, wherein said vector comprises AAV serotype 5 inverted terminal repeats (ITRs).
99. The method of any one of embodiments 69-72, wherein said vector comprises AAV serotype 9 inverted terminal repeats (ITRs).
100. The method of any one of embodiments 69-72, wherein said vector comprises at least one ITR nucleic acid sequence at least 80% identical to SEQ ID NO: 1.
101. The method of any one of embodiments 69-72, wherein said vector comprises at least one ITR nucleic acid sequence at least 80% identical to SEQ ID NO: 9.
102. The method of embodiment 69, wherein said converting occurs in the central nervous system (CNS) or peripheral nervous system.
103. The method of embodiment 71, wherein said converting occurs in the CNS.
104. The method of embodiment 71 or 72, wherein said subject in need thereof is a mammal.
105. The method of embodiment 104, wherein said mammal is a human.
106. The method of embodiment 104, wherein said mammal is a non-human primate.
107. The method of embodiment 71 or 72, wherein said delivering comprises a local administration.
108. The method of embodiment 71 or 72, wherein said delivering comprises systemic administration.
109. The method of embodiment 71 or 72, wherein said delivering comprises an administration selected from the group consisting of an intraperitoneal administration, intramuscular administration, intravenous administration, intrathecal administration, intracerebral administration, intracranial administration, intra lateral ventricle of the brain administration, intra cisterna magna administration, intra vitreous administration, intra-subretina administration, intraparenchymal administration, intranasal administration, and oral administration.
110. The method of embodiment 69 or 70, wherein said injecting comprises an injection selected from the group consisting of an intraperitoneal injection, intramuscular injection, intravenous injection, intrathecal injection, intracerebral injection, intracranial injection, intra lateral ventricle of the brain injection, intra cisterna magna injection, intra vitreous injection, intra-subretina injection, intraparenchymal injection, intranasal injection, and oral injection.
111. The method of embodiments 71 or 72, wherein said delivering comprises injecting.
112. The method of any one of embodiments 69, 70, or 111, wherein said injecting is performed at a concentration of between 10¹⁰ particles/mL and 10¹⁴ particles/mL.
113. The method of embodiment 112, wherein said injecting further comprises a flow rate of between 0.1 μL/minute and 5.0 μL/minute.
114. The method of embodiment 71, wherein said at least one glial cell is selected from the group consisting of at least one astrocyte and at least one NG2 cell.
115. The method of embodiment 69, wherein said at least one glial cell is at least one astrocyte.
116. The method of embodiment 114 or 115, wherein said at least one astrocyte is a reactive astrocyte.
117. The method of embodiment 1, wherein said neuron is a functional neuron.
118. The method of any one of embodiments 69, 70, and 117, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
119. The method of embodiment 71, wherein said subject exhibits an improvement of at least one neurological condition symptom as compared to said subject prior to said delivering.
120. The method of embodiment 119, wherein said improvement is measured within 1 year of said delivering.
121. The method of any one of embodiments 69, 70, or 111, wherein said method comprises directly injecting said AAV vector into the brain of said subject.
122. The method of any one of embodiments 69 or 70 wherein said converting is in the cerebral cortex of said brain.
123. The method of any one of embodiments 69 or 70, or 111, wherein said method comprises directly injecting said AAV vector into the spinal cord of said subject.
124. The method of embodiment 72, wherein said neurological condition comprises an injury to the central nervous system (CNS) or peripheral nervous system.
125. The method of embodiment 72, wherein said neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.
126. The method of embodiment 72, wherein said neurological condition is Alzheimer's Disease.
127. The method of embodiment 72, wherein said neurological condition is Parkinson's Disease.
128. The method of embodiment 72, wherein said neurological condition is ALS.
129. The method of embodiment 72, wherein said neurological condition is Huntington's Disease.
130. The method of embodiment 72, wherein said neurological condition is a stroke.
131. The method of embodiment 130, wherein said stroke is an ischemic stroke.
132. The method of embodiment 130, wherein said stroke is a hemorrhagic stroke.
133. The method of embodiment 72, wherein said method is capable of converting at least one glial cell into a neuron.
134. The method of embodiment 133, wherein said glial cells are selected from the group consisting of astrocytes and NG2 cells.
135. The method of embodiment 132, wherein said glial cells are astrocytes.
136. The method of embodiment 135, wherein said astrocytes are reactive astrocytes.
137. The method of embodiment 133, wherein said glial cells are GFAP positive.
138. The method of embodiment 133, wherein said neurons are functional neurons.
139. The method of embodiment 138, wherein said functional neurons are selected from the group consisting of glutamatergic neurons, GABAergic neurons, dopaminergic neurons, cholinergic neurons, seratonergic neurons, epinephrinergic neurons, motor neurons, and peptidergic neurons.
140. The method of embodiments 69 or 70, wherein a therapeutically effective dose of said AAV is injected into said subject.
141. The method of embodiments 71 or 72, wherein a therapeutically effective dose of said AAV is delivered to said subject.
142. The method of embodiment 140 or 141, wherein said therapeutically effective dose is administered with a pharmaceutically acceptable carrier.
143. An adeno-associated virus (AAV) vector comprising a human distal-less homeobox 2 (hDlx2) sequence comprising the nucleic acid sequence of SEQ ID NO: 6, wherein the hDlx2 sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
144. An adeno-associated virus (AAV) vector comprising a nucleic acid coding sequence encoding a human distal-less homeobox 2 (hDlx2) protein comprising the amino acid sequence of SEQ ID NO: 10, wherein said coding sequence is operably linked to regulatory elements comprising:
   (a) a glial fibrillary acidic protein (GFAP) promoter comprising a nucleic acid sequence selected from the group consisting of SEQ ID NOs: 3, 4, and 12;
   (b) an enhancer from a human elongation factor-1 alpha (EF1-α) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal with a nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
145. A composition comprising an adeno-associated virus (AAV) vector for converting glial cells to functional neurons in a human, wherein said AAV vector comprises a human distal-less homeobox 2 (hDlx2) sequence having a nucleic acid sequence of SEQ ID NO: 6, and wherein said sequence is operably linked to regulatory elements comprising:
   (a) a human glial fibrillary acidic protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
146. A composition comprising an adeno-associated-virus (AAV) vector for converting glial cells to functional neurons in a human, wherein said AAV vector comprises a nucleic acid sequence encoding a distal-less homeobox 2 (hDlx2) protein comprising the amino acid coding sequence of SEQ ID NO: 10, and wherein said coding sequence is operably linked to regulatory elements comprising:
   (a) a human glial fibrillary acidic protein (GFAP) promoter comprising a nucleic acid sequence selected from the group consisting of SEQ ID NOs: 3,4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal sequence comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
147. A method of converting reactive astrocytes to functional neurons in a brain of a living human comprising: injecting an adeno-associated virus (AAV) into a subject in need thereof, wherein said AAV comprises a DNA vector construct comprising a human distal-less homeobox 2 (hDlx2) sequence comprising the nucleic acid sequence of SEQ ID NO: 6, wherein said sequence is operably linked to regulatory elements comprising:
   (a) a human glial fibrillary acid protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.
148. A method of converting reactive astrocytes to functional neurons in a brain of a living brain comprising: injecting an adeno-associated virus (AAV) into a subject in need thereof, wherein said AAV comprises a DNA vector construct comprising a nucleic acid sequence encoding a human distal-less homeobox 2 (hDlx2) protein comprising the amino acid coding sequence of SEQ ID NO: 10, wherein said coding sequence is operably linked to expression control elements comprising:
   (a) a human glial fibrillary acid protein (GFAP) promoter comprising a nucleic acid sequence selected from the group consisting of SEQ ID NOs: 3, 4, and 12;
   (b) an enhancer from the human elongation factor-1 alpha (EF-1 alpha) promoter comprising the nucleic acid sequence of SEQ ID NO: 2 or a cytomegalovirus (CMV) enhancer comprising the nucleic acid sequence of SEQ ID NO: 11; and
   (c) a SV40 polyadenylation signal comprising the nucleic acid sequence of SEQ ID NO: 8, a hGH polyadenylation signal comprising the nucleic acid sequence of SEQ ID NO: 13, or a bGH polyadenylation signal comprising the nucleic acid sequence of SEQ ID NO: 20.

1. An adeno-associated virus (AAV) vector comprising a human distal-less homeobox 2 (hDlx2) sequence, wherein the hDlx2 sequence comprises a nucleic acid sequence of SEQ ID NO: 6 or a portion thereof, or wherein the hDlx2 sequence encodes an amino acid sequence of SEQ ID NO: 10 or a portion thereof, wherein the hDlx2 sequence is operably linked to regulatory elements comprising:
(a) a glial fibrillary acidic protein (GFAP) promoter;
(b) an enhancer from a human elongation factor-1 alpha (EF1-α) promoter;
(c) a chimeric intron;
(d) a woodchuck hepatitis virus posttranscriptional regulatory element (WPRE); and
(e) a SV40 polyadenylation signal, a hGH polyadenylation signal, or a bGH polyadenylation signal.

2. The AAV vector of claim 1, wherein:
(a) the glial fibrillary acidic protein (GFAP) promoter comprises a nucleic acid sequence at least 80% identical to a sequence selected from the group consisting of SEQ ID NOs: 3, 4, and 12;
(b) the enhancer from a human elongation factor-1 alpha (EF1-α) promoter comprises a nucleic acid sequence at least 80% identical to SEQ ID NO: 2 or the cytomegalovirus (CMV) enhancer comprises a nucleic acid sequence at least 80% identical to SEQ ID NO: 11;
(c) the chimeric intron comprises a nucleic acid sequence at least 80% identical to a sequence selected from the group consisting of SEQ ID NOs: 5 and 19;
(d) the woodchuck hepatitis virus posttranscriptional regulatory element (WPRE) comprises a nucleic acid sequence at least 80% identical to a sequence selected from the group consisting of SEQ ID NOs: 7 and 18; or
(e) the SV40 polyadenylation signal comprises a nucleic acid sequence at least 80% identical to SEQ ID NO: 8, the hGH polyadenylation signal comprises a nucleic acid sequence at least 80% identical to SEQ ID NO: 13, or the bGH polyadenylation signal comprises a nucleic acid sequence at least 80% identical to SEQ ID NO: 20.

3. (canceled)

4. A composition comprising an adeno-associated virus (AAV) vector for converting a glial cell to a functional neuron in a subject in need thereof, wherein said AAV vector comprises a human distal-less homeobox 2 (hDlx2) sequence, wherein said hDlx2 comprises a nucleic acid sequence of SEQ ID NO: 6 or a portion thereof, or wherein said hDlx2 sequence comprises a nucleic acid sequence encoding an amino acid sequence of SEQ ID NO: 10 or a portion thereof, and wherein said hDlx2 sequence is operably linked to regulatory elements comprising:
(a) a human glial fibrillary acidic protein (GFAP) promoter;
(b) an enhancer from the human elongation factor-1 alpha (EF-1 alpha) promoter or a cytomegalovirus (CMV) enhancer;
(c) a chimeric intron;
(d) a woodchuck hepatitis virus posttranscriptional regulatory element (WPRE); and
(e) a SV40 polyadenylation signalk, a hGH polyadenylation signal, or a bGH polyadenylation signal.

5. The composition of claim 4, wherein:
(a) the human glial fibrillary acidic protein (GFAP) promoter comprises a nucleic acid sequence at least 80% identical to a sequence selected from the group consisting of SEQ ID NOs: 3,4, and 12;
(b) the enhancer from the human elongation factor-1 alpha (EF-1 alpha) promoter comprises a nucleic acid sequence at least 80% identical to SEQ ID NO: 2 or the cytomegalovirus (CMV) enhancer comprises a nucleic acid sequence at least 80% identical to SEQ ID NO: 11;
(c) the chimeric intron comprises a nucleic acid sequence at least 80% identical to a sequence selected from the group consisting of SEQ ID NOs: 5 and 19;
(d) the woodchuck hepatitis virus posttranscriptional regulatory element (WPRE) comprises a nucleic acid sequence at least 80% identical to a sequence selected from the group consisting of SEQ ID NOs: 7 and 18; or
(e) the SV40 polyadenylation signal sequence comprises a nucleic acid sequence at least 80% identical to SEQ ID NO: 8, the hGH polyadenylation signal comprises a nucleic acid sequence at least 80% identical to SEQ ID NO: 13, or the bGH polyadenylation signal comprises a nucleic acid sequence at least 80% identical to SEQ ID NO: 20.

6. (canceled)

7. The AAV vector of claim 1, wherein said AAV vector is selected from the group consisting of AAV serotype 2, AAV serotype 5, and AAV serotype 9.

8.-15. (canceled)

16. The AAV vector of claim 1, wherein said hDlx2 sequence comprises a nucleic acid coding sequence encoding an amino acid sequence at least 80% identical or similar to SEQ ID NO: 10.

17. The AAV vector of claim 1, wherein said hDlx2 sequence comprises a nucleic acid sequence at least 80% identical to SEQ ID NO: 6, or a complement thereof.

18.-36. (canceled)

37. The AAV vector of claim 1, wherein said AAV vector comprises at least one ITR nucleic acid sequence at least 80% identical to SEQ ID NO: 9.

38.-43. (canceled)

44. The composition of claim 4, wherein said subject has a neurological condition selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.

45.-52. (canceled)

53. The composition of claim 4, wherein said glial cell is selected from the group consisting of an astrocyte, a reactive astrocyte, and an NG2 cell.

54.-57. (canceled)

58. The composition of claim 4, wherein said functional neuron is selected from the group consisting of glutamatergic neurons, GABAergic neurons, dopaminergic neurons, cholinergic neurons, seratonergic neurons, epinephrinergic neurons, motor neurons, and peptidergic neurons.

59.-68. (canceled)

69. A method of (i) converting a glial cell to a neuron in a subject in need thereof, (ii) treating a neurological condition in a subject in need thereof, or (iii) converting a reactive astrocyte to a neuron in a subject in need thereof, the method comprising: delivering a composition to said subject in need thereof, wherein said composition comprises an adeno-associated virus (AAV) vector comprising a human distal-less homeobox 2 (hDlx2) sequence operably linked to regulatory elements comprising:
(a) a human glial fibrillary acid protein (GFAP) promoter;
(b) an enhancer from the human elongation factor-1 alpha (EF-1 alpha) promoter or a cytomegalovirus (CMV) enhancer;
(c) a chimeric intron;
(d) a woodchuck hepatitis virus posttranscriptional regulatory element (WPRE); and
(e) a SV40 polyadenylation signal, a hGH polyadenylation signal, or a bGH polyadenylation signal.

70. The method of claim 69, wherein:
(a) the human glial fibrillary acid protein (GFAP) promoter comprises a nucleic acid sequence at least 80% identical to a sequence selected from the group consisting of SEQ ID NOs: 3, 4, and 12;
(b) the enhancer from the human elongation factor-1 alpha (EF-1 alpha) promoter comprises a nucleic acid sequence at least 80% identical to SEQ ID NO: 2 or the cytomegalovirus (CMV) enhancer comprises a nucleic acid sequence at least 80% identical to SEQ ID NO: 11;
(c) the chimeric intron comprises a nucleic acid sequence at least 80% identical to a sequence selected from the group consisting of SEQ ID NOs: 5 and 19;
(d) the woodchuck hepatitis virus posttranscriptional regulatory element (WPRE) comprises a nucleic acid sequence at least 80% identical to a sequence selected from the group consisting of SEQ ID NOs: 7 and 18; or
(e) the SV40 polyadenylation signal comprises a nucleic acid sequence at least 80% identical to SEQ ID NO: 8, the hGH polyadenylation signal comprises a nucleic acid sequence at least 80% identical to SEQ ID NO: 13, or the bGH polyadenylation signal comprises a nucleic acid sequence at least 80% identical to SEQ ID NO: 20.

71.-79. (canceled)

80. The method of claim 69, wherein said hDlx2 sequence comprises a nucleic acid sequence encoding an amino acid sequence at least 80% identical or similar to SEQ ID NO: 10.

81. The method of claim [[78]]69, wherein said hDlx2 sequence comprises a nucleic acid sequence at least 80% identical to SEQ ID NO: 6, or a complement thereof.

82.-113. (canceled)

114. The method of claim 69, wherein said glial cell is selected from the group consisting of an astrocyte, a reactive astrocyte, and an NG2 cell.

115.-117. (canceled)

118. The method of claim 69, wherein said neuron is selected from the group consisting of glutamatergic neurons, GABAergic neurons, dopaminergic neurons, cholinergic neurons, seratonergic neurons, epinephrinergic neurons, motor neurons, and peptidergic neurons.

119. The method of claim 69, wherein said subject exhibits an improvement of at least one neurological condition symptom as compared to said subject prior to said delivering.

120.-123. (canceled)

124. The method of claim 69, wherein said neurological condition comprises an injury to the central nervous system (CNS) or peripheral nervous system.

125. The method of claim 69, wherein said neurological condition is selected from the group consisting of Alzheimer's Disease, Parkinson's Disease, amyotrophic lateral sclerosis (ALS), Huntington's Disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.

126.-148. (canceled)


SEQUENCE LISTING (21 sequences)

SEQ ID NO: 1 - DNA, 128 bp, Artificial Sequence
  Description of Artificial Sequence Synthetic polynucleotide
tgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 2 - DNA, 153 bp, Artificial Sequence
  Description of Artificial Sequence Synthetic polynucleotide
tgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggca

SEQ ID NO: 3 - DNA, 686 bp, Artificial Sequence
  Description of Artificial Sequence Synthetic polynucleotide
ctagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 4 - DNA, 1667 bp, Artificial Sequence
  Description of Artificial Sequence Synthetic polynucleotide
ctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagag

SEQ ID NO: 5 - DNA, 133 bp, Artificial Sequence
  Description of Artificial Sequence Synthetic polynucleotide
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - DNA, 987 bp, Homo sapiens
atgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctga

SEQ ID NO: 7 - DNA, 589 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 251 bp, Artificial Sequence
  Description of Artificial Sequence Synthetic polynucleotide
cgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 9 - DNA, 139 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca

SEQ ID NO: 10 - protein, 328 aa, Homo sapiens
MTGVFDSLVADMHSTQIAASSTYHQHQQPPSGGGAGPGGNSSSSSSLHKPQESPTLPVSTATDSSYYTNQQHPAGGGGGGGSPYAHMGSYQYQASGLNNVPYSAKSSYDLGYTAAYTSYAPYGTSSSPANNEPEKEDLEPEIRIVNGKPKKVRKPRTIYSSFQLAALQRRFQKTQYLALPERAELAASLGLTQTQVKIWFQNRRSKFKKMWKSGEIPSEQHPGASASPPCASPPVSAPASWDFGVPQRMAGGGGPGSGGSGAGSSGSSPSSAASAFLGNYPWYHQTSGSASHLQATAPLLHPTQTPQPHHHHHHHGGGGAPVSAGTIF

SEQ ID NO: 11 - DNA, 380 bp, Artificial Sequence
  Description of Artificial Sequence Synthetic polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 12 - DNA, 2214 bp, Homo sapiens
cgcgtcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggg

SEQ ID NO: 13 - DNA, 477 bp, Homo sapiens
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 14 - DNA, 241 bp, Artificial Sequence
  Description of Artificial Sequence Synthetic polynucleotide
gtcctttccacaagatatataaacccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctc

SEQ ID NO: 15 - DNA, 58 bp, Artificial Sequence
  Description of Artificial Sequence Synthetic oligonucleotide
ccggtggttcagttacgggttaattctcgagaattaacccgtaactgaaccatttttg

SEQ ID NO: 16 - DNA, 60 bp, Artificial Sequence
  Description of Artificial Sequence Synthetic oligonucleotide
ccggcagttacgggttaattaatacctgacccatattaattaacccgtaactgctttttg

SEQ ID NO: 17 - DNA, 57 bp, Artificial Sequence
  Description of Artificial Sequence Synthetic oligonucleotide
ccggtgttgccgcagcatcactaatctcgagattagtgatgctgcggcaacattttg

SEQ ID NO: 18 - DNA, 581 bp, Artificial Sequence
  Description of Artificial Sequence Synthetic polynucleotide
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 19 - DNA, 1062 bp, Artificial Sequence
  Description of Artificial Sequence Synthetic polynucleotide
ggagtcgctgcgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaacgtgctggttgttgtgctgtctcatcattttggcaaagat

SEQ ID NO: 20 - DNA, 208 bp, Artificial Sequence
  Description of Artificial Sequence Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 21 - DNA, 66 bp, Artificial Sequence
  Description of Artificial Sequence Synthetic oligonucleotide
ggaagcggagctactaacttcagcctgctgaagcaggctggagacgtggaggagaaccctggacct